(19) United States
(12) Patent Application Publication
(10) Pub. No.: US 2011/0177054 A1
(43) Pub. Date: Jul. 21, 2011

(54) USE OF ENDO-LYSOSOMAL SYSTEM AND SECRETED VESICLES (EXOSOME-LIKE) IN TREATMENTS AND DIAGNOSTICS BASED ON SMALL RNA AND EXPERIMENTAL STUDY OF SMALL RNA

(76) Inventors: Derrick Gibbings, Strausbourg (FR); Olivier Voinnet, Strausbourg (FR)

(21) Appl. No.: 12/996,287

(22) PCT Filed: Jun. 5, 2009
(86) PCT No.: PCT/IB09/05878

§ 371 Date: Mar. 23, 2011

Related U.S. Application Data
(60) Provisional application No. 61/059,354, filed on Jun. 6, 2008.
(60) Provisional application No. 61/153,324, filed on Feb. 18, 2009.

(51) Int. Cl. A61K 38/44; C12Q 1/68; C40B 30/04; G01N 33/53; C40B 30/00; C12N 5/07; A61K 31/713; A61K 31/7088; A61K 38/02; A01N 37/18; A01N 43/08; A61K 38/45; A61K 38/46; A61P 3/00

(52) U.S. Cl. 424/94.4; 435/6.1; 435/6.12; 506/9; 436/501; 506/7; 435/6.11; 435/375; 514/44 A; 514/44 R; 514/1.1; 424/94.5; 424/94.6

(57) ABSTRACT

The present invention relates to a method for determining the delivery rates and/or efficiency of a siRNA, miRNA or related molecule to target organs or cells, a kit and the use of proteins or lipids involved in the formation of the endolysosomal system for modulating the activity and/or the cell-to-cell transfer of RNA, small RNA, for example miRNA, siRNA and piRNA, mRNA or non-coding RNA.

It finds many applications in particular in methods for identifying the target(s) of miRNA or siRNA therapeutics, in methods for determining the efficiency of a treatment with siRNA and/or miRNA therapeutics, in methods for determining the efficiency of a treatment with siRNA and/or miRNA therapeutics, and in methods for genotyping and/or characterizing the condition of a person, a tumor or a fetus.

### TECHNICAL FIELD

The present invention relates to a method for determining the delivery rates and/or efficiency of a siRNA (short interfering ribonucleic acid), miRNA (micro-ribonucleic acid) or related molecule to target organs or cells, a kit and the use of proteins or lipids involved in the formation of the endolysosomal system for modulating the activity and/or the cell-to-cell transfer of RNA (ribonucleic acid), small RNA, for example miRNA, siRNA and piRNA (Piwi-interacting ribonucleic acid, mRNA (messenger ribonucleic acid) or non-coding RNA.

Among the invention's many applications we can cite in particular methods for identifying the target(s) of miRNA or siRNA therapeutics, methods for determining the efficiency of a treatment with siRNA and/or miRNA therapeutics, and methods for genotyping and/or characterizing the condition of a person, a tumor or a fetus.

In the following description that follows, the references refer to the attached reference list.

All the documents cited herein in the reference list are incorporated by reference in the text below.

### STATE OF THE ART

The endosomal sorting complex required for transport (ESCRT) complex located on the cytoplasmic surface of the multivesicular body (MVB) recognizes and sorts ubiquitinated proteins into vesicles which bud into the MVB and can be delivered to the lysosome or released into the extracellular space as exosomes. While the RNA silencing machinery is often stated to be independent of membranes, other evidence has suggested that Ago2 (Argonaute-2), at least, may be closely associated with unidentified membranes. GW182 contains an ubiquitin-binding domain and is ubiquitinated. The SiRNA targeting several members of the ESCRT complex blocked miRNA activity, but did not grossly disrupt localization of GW182 to the MVB. GW182 was distinctly enriched in exosomes, which also contained miRNA. Finally, exosomes transferred miRNA activity to target cells in a BIG2-dependent manner (Brefeldin A-inhibited guanine nucleotide-exchange protein 2)

The multivesicular body (MVB) is an intermediate sorting centre between endosomes and lysosomes that contains intraluminal vesicles (ILV) formed by inward budding. One of the most studied mechanisms of delivery of proteins into the MVB is the Endosomal Sorting Complex Required for Transport (ESCRT), which recognizes ubiquitinated proteins and delivers them into ILV. Ubiquitinated proteins and factors associated to them can be sorted into MVB by three heteromeric subcomplexes collectively termed ESCRT, to be further secreted in exosomes and/or degraded via the lysosome. ILV may be parlayed to the lysosome for degradation. Alternatively the MVB may fuse with the plasma membrane to release ILV into the extracellular space where ILV are termed exosomes. While the release of exosomes has been mostly studied in monocytes, dendritic cells and some tumor cells, most cells appear to release exosomes. Exosomes can transfer proteinaceous antigen from a tumor cell to a dendritic cell to activate an anti-tumor immune response. It is not known whether transfer of peptide antigens occurs through endocytosis and degradation of proteins or through cytoplasmic delivery of antigen. Exosomes also contain plasma membrane receptors on their surface derived from the producing cell, which allow them to be targeted to specific cell types, and even to activate plasma membrane receptors on the target cell.

Proteins and RNA of the cytoplasm, the cellular membrane, other organelles such as the Golgi, or the extracellular space can be delivered to the MVB, either by ubiquitination of a protein or by domains that associate to other proteins, RNA or lipids sorted into the MVB. The MVB can then transfer these proteins to the cellular membrane, secrete them in vesicles named exosomes, or deliver them for degradation in lysosomes. Thus in the present invention small RNA and proteins essential for the function of miRNA have been found in exosomes secreted by the MVB.

RNA consisting of 18 to 35 nucleotides named miRNA, siRNA or piRNA can modulate expression of genes or non-coding RNAs by the formation of heterochromatin, or other modifications resulting in changes of DNA transcription (DNA [deoxyribonucleic acid]), degradation or stabilization of mRNA, or inhibition or activation of the translation of mRNA into proteins. These small RNA play a role in the development, cancer and immunity of many of organisms. Moreover, the technology of siRNA has been adapted to specifically inhibit the expression of an RNA or protein in research and medicine. MicroRNA (miRNA) are 19 to 24 nucleotide RNA molecules that sequence-specifically can inhibit or activate translation or promote degradation or stabilization and localization of a targeted mRNA. Several hundreds of miRNA are believed to regulate about 30% of genes. A few years ago, subcellular structures named P-bodies or GW-bodies have been identified as being able to congregate small RNA and proteins essential for the function thereof, for example many of the proteins involved in post-transcriptional regulation mediated by miRNA and siRNA, including Dcp1a (Decapping Enzyme Homolog A), GW182, and Argonaute family members. P-bodies are believed to be independent of lipid bilayers (Eystathioy, T. et al. A phosphorylated cytoplasmic autoantigen, GW182, associates with a unique population of human mRNAs within novel cytoplasmic speckles. Mol. Biol. Cell. 13, 1338-1351 (2002) [1], Schneider, M. D. et al. Gawky is a component of cytoplasmic mRNA processing bodies required for early Drosophila development. J. Cell Biol. 174, 349-358 (2006) [2]) and do not overlap extensively with any known intracellular organelle or structure including lysosomes, early endosomes, Golgi, or peroxisomes ([1], Jakymiw, A. et al. Disruption of GW bodies impairs mammalian RNAinterference. Nat. Cell Biol. 7, 1267-1274 (2005) [3]). Nonetheless, some evidence suggests components of the RNA silencing machinery may be associated with membranes. Ago2 purifies with microsomes and microsomal Ago2 is accessible to trypsin digestion only after treatment with detergents (Cikaluk, D. E. et al. GERp95, a membrane-associated protein that belongs to a family of proteins involved in stem cell differentiation. Mol. Biol. Cell. 10, 3357-3372 (1999) [4]). Furthermore, autoantibodies against the lipid phosphatidylethanolamine that gave a staining pattern identical to autoantibodies recognizing GW182 were recently identified (Laurino, C. C. et al. Human autoantibodies to diacyl-phosphatidylethanolamine recognize a specific set of discrete cytoplasmicdomains. Clin. Exp. Immunol. 143, 572-584 (2006) [5]). Surprisingly, GW182 has an ubiquitin-associated (UBA) domain suggesting it may be linked to ESCRT complexes at the MVB by ubiquitinated proteins.

Moreover many proteins of the ESCRT which bind to ubiquitinated proteins and sort ubiquinated proteins in the MVB have also been found to be important in helping a miRNA or a siRNA to reduce expression of its mRNA and its target proteins. Exosomes have also been found to be able to transfer miRNA or siRNA activity from one cell to another. Furthermore since many proteins involved in the formation of the MVB are important for cytokinesis and for the regulation of transcription or gene expression by DNA heterochromatin, some of these proteins may be able to extend the siRNA effect by several cellular divisions or to allow small RNA to affect DNA structure and/or transcription such as through heterochromatin formation or epigenetic regulation.

Exosomes are targeted to macrophages and DC (dendritic cells) by specific exosome receptors such as ICAM-1 (Inter-Cellular Adhesion Molecule 1) (Segura, E., Guerin, C., Hogg, N., Amigorena, S., & Thery, C. CD8+ dendritic cells use LFA-1 to capture MHC-peptide complexes from exosomes in vivo. J. Immunol. 179, 1489-1496 (2007) [36]) and possibly MFG-E8 (Milk fat globule-EGF factor 8 protein) (Zeelenberg, I. S. et al. Targeting tumor antigens to secreted membrane vesicles in vivo induces efficient antitumor immune responses. Cancer Res. 68, 1228-1235 (2008) [37]). Exosomes may be transported in lymph, pleural spaces, or blood to distant antigen presenting cells so as to regulate immune responses (Morelli, A. E. et al. Endocytosis, intracellular sorting, and processing of exosomes by dendritic cells. Blood. 104, 3257-3266 (2004) [38]). Transport of immunosuppressive miR-146 and -155 (O'Connell, R. M., Taganov, K. D., Boldin, M. P., Cheng, G., & Baltimore, D. MicroRNA-155 is induced during the macrophage inflammatory response. Proc. Natl. Acad. Sci. U.S.A. 104, 1604-1609 (2007) [39]) by exosomes secreted by activated DC at a site of pathogen infection to DC in lymph nodes or spleen may help establish a peripheral border for the immune response, and reduce the risk of systemic activation and septic shock. A particularly intriguing possibility relates to the subversion by many viruses or bacteria of RNA processing or translational machinery to accomplish their life cycle (Pelchen-Matthews, A., Raposo, G., & Marsh, M. Endosomes, exosomes and Trojan viruses. Trends Microbiol. 12, 310-316 (2004) [40]). Routinely packaging RNA and affixed proteins into exosomes for transport to antigen presenting cells would be an ingenious way to generate immune responses to key viral proteins that may otherwise evade protein-based antigen specific immunity in mammals. Exosomes produced by each type of differentiated cell, by containing a specialized set of surface receptors derived from that cell, may enact similar regulatory changes at a distance, partly through miRNA. For example, exosomes share many similarities with melanosomes which traffick skin and hair color regulating elements, or epididymosomes (Sullivan, R., Saez, F., Girouard, J., & Frenette, G. Role of exosomes in sperm maturation during the transit along the male reproductive tract. Blood Cells Mol. Dis. 35, 1-10 (2005) [41]) which are involved in the maturation of spermatozoa. More distantly, exosomes share similarities with synaptic vesicles and various granules secreted by immune cells. One could speculate that synapse-axon junctions could be feedback regulated by miRNA exchange of synaptic vesicles, or that a subset of miRNA could be specifically triaged into immune cell granules and delivered into parasites, tumorogenic, or virally infected cells to target genes essential for their survival. Intriguingly in this regard, a series of electron microscopy studies suggest the presence of RNA in mast cell granules (Dvorak, A. M. & Morgan, E. S. The case for extending storage and secretion functions of human mast cell granules to include synthesis. Prog. Histochem. Cytochem. 37, 231-318 (2002) [27]).

Retroviruses like HIV-1 (Human immunodeficiency virus-1) co-opt the MVB for packaging and intercellular trafficking (Martin-Serrano, J. The role of ubiquitin in retroviral egress. Traffic. 8, 1297-1303 (2007) [42]). Conservation of Gag in endogenous retroviruses and the discovery of Gag from endogenous retroviruses in proteomics studies of exosomes (Segura, E., Amigorena, S., & Thery, C. Mature dendritic cells secrete exosomes with strong ability to induce antigen-specific effector immune responses. Blood Cells Mol. Dis. 35, 89-93 (2005) [43]) suggests some endogenous retroviruses may also take advantage of the MVB. Inclusion of miRNA, some of which can suppress retroviruses (Lecellier, C. H. et al. A cellular microRNA mediates antiviral defense in human cells. Science. 308, 557-560 (2005) [44]), and potentially of siRNA targeting activated endogenous retroviruses and other transposable elements in exosomes may be a cellular and cell-autonomous counterdefense against these agents of evolution. Moreover, bacteria with type III secretion systems inject proteins into cells that target components of the RNA silencing machinery (Suppression of the microRNA pathway by bacterial effector proteins Navarro et al. Science 2008 321: 964 [87]). Some intracellular bacteria, such as Mycobacteria smegmatis are limited by the ESCRT complex, and perhaps directly or indirectly affect the RNA silencing machinery (Philips, J. A., Porto, M. C., Wang, H., Rubin, E. J., & Perrimon, N. ESCRT factors restrict mycobacterial growth. Proc. Natl. Acad. Sci. U.S.A. 105, 3070-3075 (2008) [45]).

siRNA and miRNA are of great interest for pharmaceutical targeting of a specific gene with minimal toxicity and endogenous miRNA play an important role in many cancers, developmental deficiencies and immune responses. But the delivery of siRNA or miRNA into a majority of all target cells is a major problem limiting their use in vivo. Moreover many genes directly regulating miRNA activity are essential to cell survival, and are thus not easy to target with drugs. Furthermore, there is often no treatment which eliminates pathogens going through the MVB and/or pathogens that associate with the MVB sometimes develop resistance to current drugs. Finally, the effect of siRNA or miRNA is often less than expected for an effective inhibition of the protein production.

Thus, a need persists for the development of improved methods of delivery of siRNA or miRNA into a majority of all target cells in vivo, having no or minor indesirable effects on cells, or for targeting genes directly regulating miRNA activity. Furthermore, there is still a need for treatment which eliminates pathogens going through the MVB and/or pathogens that associate with the MVB sometimes develop resistance to current drugs.

### DISCLOSURE OF THE INVENTION

The inventors of the present invention have now discovered, entirely unexpectedly, that a significant proportion of GW182 and Ago2 associate with the multivesicular body (MVB) in P-body-like structures, from where miRNA may be trafficked intercellularly. The association of RNA silencing machinery with the MVB is functional as some components of the ESCRT complex are important for the activity of miRNA. As such, this study proposes a new link between an intracellular compartment and the miRNA pathway.

The inventors also discovered that GW-bodies containing GW182 and Ago2 are distinct from P-bodies because they congregate with MVB. Moreover, miRNA-repressed mRNAs are specifically enriched at cellular membranes, meaning that MVB are novel sites of miRNA-RISC action. Purified exosome-like vesicles secreted by MVB are dramatically enriched in GW182, but not P-body components. A small fraction of cellular Ago2 and mature miRNA are also found in exosomes, but miRNA-repressed mRNAs are absent. Consistent with its ESCRT-dependent sorting into MVB, GW182, but not P-body components, is ubiquitinated or interact with ubiquitinated proteins. Moreover, cells depleted of some ESCRT components over-accumulate GW182 and display compromised miRNA-mediated gene silencing. Therefore, GW182, possibly in association with a fraction of miRNA-loaded Ago2, is sorted into MVB for secretion and/or lysosomal degradation. This process allows high dissociation rates of membrane-bound miRNA-RISC, required for multiple rounds of mRNA repression. Alternatively, this process may be involved in loading of Ago with small RNA, in regulating small RNA biogenesis, target recognition, or turnover/degradation of small RNA complexes. Indeed, we show that mRNA targets of miRNA accumulate on endolysosomal membranes, meaning that mRNA target recognition may occur on these membranes. We find GW182 in density gradient fractions containing lysosomes, suggesting that turnover/degradation of small RNA complexes occurs via the lysosome.

The inventors have also demonstrated that several key components of the RNA silencing machinery including Ago2 and GW182 co-localize with markers of the MVB.

Surprisingly, the inventors also found that the ESCRT complex and ubiquitination are important regulatory components of RNA silencing and its intercellular transfer in mammalian cells.

The inventors also demonstrate that a large quantity of P-bodies or GW-bodies associated with the multivesicular body (MVB), is associated with and potentially surrounded by a membrane. Moreover it has been shown that many proteins involved in the formation of the MVB, namely proteins from the endosomal sorting complex required for transport (ESCRT) are important for the miRNA activity, such as Alix, Hrs (hepatocyte responsive serum phosphoprotein), vps36 (vacuolar protein sorting associated protein 36, EF1a1 and 2, PRP (Prion Protein), HMGCR (HMG CoA Reductase), sphingomyelinases (targeted by the chemical inhibitor GW4869), NPC1 (Niemann-Pick C1 protein) or other proteins associated with the MVB or the secretion of exosomes (e.g. BIG2). The ESCRT complex can inhibit or enhance the capacity of a miRNA to suppress the protein expression of its complementary mRNA. The ESCRT complex may provide means to selectively modulate the function of one type of small RNA while leaving another version's function intact. For example, miRNA activity may be affected but not siRNA activity. It has also been shown that little vesicles (of about 50 nm in diameter), named exosomes, formed in the MVB and released by cells can transfer miRNA or siRNA activity from one cell to another cell. These discoveries show the means by which miRNA or siRNA activity can be modulated in a cell and transferred to other human cells. Furthermore, the inventors find that knockdown of some members of the ESCRT complex ablates miRNA activity, while knockdown of others increases its activity. Finally, components of the RNA silencing machinery and miRNA are loaded into exosomes and can inhibit gene expression in cells incubated with exosomes.

Therefore, a first aspect of the invention relates to a method for determining the delivery rates and/or efficiency of a siRNA, miRNA or related molecule, or inhibitors of such molecules to target organs or cells, comprising the measurement of levels, in the exosomes or vesicles of said target organs or cells, of said siRNA and/or miRNA and/or of mRNA targeted by said miRNA and/or by said siRNA.

In other words, the invention relates to a method for determining the delivery rates and/or delivery efficiency of a siRNA, miRNA or related molecule or inhibitor thereof to target organs or cells, comprising the measurement of levels, in the exosomes or vesicles of said target organs or cells, of said siRNA and/or miRNA and/or of mRNA targeted by said miRNA and/or by said siRNA.

By “siRNA” in the sense of the present invention is meant any interfering RNA that may be suitable for the invention. More particularly, it may designate a short interfering RNA comprising from 6 to 29 nucleotides. More prescisely, it may designate a short interfering RNA comprising approximately 22 nucleotides in length. Advantageously, it may be a short interfering RNA that, possibly in concert with at least one component of the RNA silencing complex containing any of AGO proteins 1 through 4 and/or GW182 proteins A, B or C or TNGW1 (trinucleotide GW1), modifies gene expression, for example through mRNA cleavage, degradation or inhibition of translation.

By “miRNA” in the sense of the present invention is meant any miRNA that may be suitable for the invention. Advantageously, it may be any naturally occurring, small non-coding RNAs that are about 17 to about 25 nucleotide bases in length in their biologically active form. Preferably, it may be as little as 9 nucleotides; i.e. comprising 9 nucleotides or less. Advantageously, miRNAs may post-transcriptionally regulate gene expression by repressing or activating target mRNA translation or promoting mRNA degradation, stabilization or subcellular localization. miRNA may be endogenously expressed or may be administered in synthetic forms and variants, in the sense of the invention, to function, often but not exclusively as negative regulator of mRNA translation, i.e. greater amounts of a specific miRNA will correlate with lower levels of target gene expression. miRNA may also activate translation (e.g. of Vasudevan refs 74 and 75). Advantageously, small RNA molecules that are miRNA-like may activate or repress transcription.

By “related molecule” in the sense of the present invention, is meant any nucleotides, including DNA (deoxyribonucleic acid), piRNA, synthetic nucleotides, and modified variants of siRNA, miRNA, DNA or a variant thereof. Such a variant may be chemically synthesised and may have advantages for RNA silencing-related processes. Some of these modifications may help protect the siRNA-related molecule from degradation, such as a 2′-o-methyl, 2′-o-allyl, 2′-deoxy-fluorouridine modification, or phosphorothioates. Other modifications may also help increase the affinity of the siRNA-related molecule for its target or reduce its off-target effects, such as the locked-nucleic acid modification, in which a methylene bridge connects the 2′-oxygen with the 4′-carbon of the ribose ring. Other modifications may enhance the loading of the correct strand of a siRNA or miRNA into AGO, such as by adding a 5′ phosphate or methyl to one strand of a doublestranded miRNA/miRNA*complex.

In the sense of the invention, siRNA, miRNA or related molecule are administered to an animal, preferably a human being, or to a cell. The human being may be a patient in need thereof. The administration may be carried out within a treatment of a disease, for example cancer, or by transfection to a cell. Once administered, this siRNA, miRNA or related molecule is delivered to target organs or cells.

The administration of siRNA, miRNA or related molecule is carried out before the measurement of the levels, in the exosomes or vesicles of the target organs or cells, of the siRNA and/or miRNA and/or mRNA targeted by the miRNA and/or by the siRNA.

This administration may be carried out by all the techniques well known by the man skilled in the art, such as mixing siRNAs with cationic lipid transfection reagents used for in vitro transfection and directly injecting the siRNA-lipid complexes into the relevant tissue or instilling it into the body cavity, or mixing siRNAs with other molecules known to carry nucleic acids into cells (i.e. certain cationic peptides), rapid retrograde injection via catheter into the draining vein, hydrodynamic injection into a peripheral vein, complexing siRNAs to cationic polymers or peptides or incorporating siRNAs into nanoparticles or liposomes, covalently or noncovalently linking to antibody fragments or ligands to cell surface receptors to limit the delivery of the siRNAs to cells that bear the specific receptor, this list not being limitative.

As all cells have the RNAi machinery and any gene is a potential target, any disease caused by or greatly exacerbated by the expression of a dominant gene can in principle be treated by RNAi. These diseases may be for example cancer, neurodegenerative disease, viral infection, and macular degeneration, this list not being limitative.

By “delivery rates”, in the sense of the present invention, is meant any ratio, or quantity, of siRNA or miRNA, administered to an individual, that may arrive at its site of action, i.e. at the site where its mRNA target is localized, or that may cleave or result in degradation of targeted mRNA, or that may inhibit translation of targeted mRNA, compared to a control.

By “delivery efficiency”, in the sense of the present invention, is meant any ratio, or quantity, of siRNA or miRNA, administered to an individual, that may have an activity at its site of action, i.e. at the site where its mRNA target is localized, or that may cleave or may result in degradation of targeted mRNA, or that inhibits translation of targeted mRNA, compared to a control.

By “target organs or cells”, in the sense of the invention, is meant any organs or cells in which the siRNA, the miRNA or related molecule may repress the translation of some mRNA, may enhance its degradation or may improve the cleavage of some mRNA.

Any cell, group of cells, cell fragment, or cell product can be used with the method of the invention.

The cell can be contained in a culture medium, or in a biological fluid, or in a bodily fluid.

By “vesicles” or “exosomes”, in the sense of the present invention, is meant any vesicles or membrane bound structures of 20-250 nm in size. Examples of such vesicles may be microvesicles, microparticles, exosome-like vesicles, dexosomes, texosomes, prostasomes, epididymosomes, “exosome-like vesicles”, this list not being exhaustive. The size of vesicle is generally of 20-250 nm, for example of 20-100 nm but may also be 100 nm-3 microM. The vesicles may be purified by means known by the man skilled in the art, for example from blood, urine, saliva and other bodily fluids. For example, it is possible to purify vesicles by elimination of cells, usually by centrifugation, for example at 200 g, thus obtaining a supernantant containing vesicles or exosomes. Another way to obtain vesicles or exosomes is performing further centrifugation steps to purify exosomes or vesicles and possibly including steps at 1000 g, and 10-16 000 g to further eliminate bigger vesicles. Subsequent centrifugation at 70-120 000 g is standardly used to purify exosome-like vesicles. Another way to obtain exosomes or vesicles include using combinations of filters that exclude different sizes of particles, for example 0.45 microM or 0.22 microM filters can be used to eliminate vesicles or particles bigger than the vesicles of interest. Exosomes or vesicles may be purified by several means, including antibodies, lectins, or other molecules that specifically bind vesicles of interest, eventually in combination with beads (e.g. agarose/sepharose beads, magnetic beads, or other beads that facilitate purification) to enrich for exosome-like vesicles. Examples of proteins enriched on exosome-like vesicles may include, but are certainly not limited to: CD63, Transferrin receptor, sialic acid, mucins, Tsg101(Tumor susceptibility gene 101), Alix, annexin II, EF1a (Translation elongation factor 1a), CD82 (Cluster of Differentiation 82), ceramide, sphingomyelin, lipid raft markers, PRNP (PRioN Protein). In the case of the invention, a marker derived from the cell type of interest may often be used. For example, if an RNAi treatment is aimed at liver tissues, vesicles may be purified from cell-free fluids using a liver-specific marker, to distinguish liver derived vesicles from vesicles derived from other cells or tissues. Other techniques to purify exosomes include density gradient centrifugation (e.g. sucrose or optiprep gradients), electric charge separation. All these enrichment and purification techniques may be combined with other methods or used by itself. Thus, exosomes isolated from bodily fluids may provide a quantitative measure of delivery rates or efficiencies of siRNA therapeutics.

By “exosome”, in the sense of the invention, is meant any small vesicles of a cell. In the sense of the present invention, such small vesicles may be generated in the cell by several means, including but not limited to, by multivesicular bodies.

By “multivesicular body”, in the sense of the invention, is meant any body of the endolysosomal system, for example any sub-type of multivesicular bodies, that may use different protein sorting mechanisms. For example, a MVB may be an endosome, for example a late endosome, MHC (major histocompatibility complex) class II loading compartment, intracellular organelles including autophagosomes, lysosomes, endosomes, and vesicles derived from the endoplasmic reticulum and Golgi which may traffick to or from the endolysosomal system.

By “measurement”, in the sense of the present invention, is meant any analysis that may allow a quantitative or qualitative measurement, or any analysis that may allow to compare levels of siRNA and/or miRNA and/or mRNA target. Any method known by the man skilled in the art for measuring RNA may be suitable for the invention. Such methods may be qRT-PCR (Quantitative Reverse Transcription polymerase chain reaction) in the many variants in practice (for example SybrGreen, Beacon technologies), or techniques based on the hybridization of oligonucleotide or nucleotide of any length with any variety or combination of modifications (notably locked nucleic acid [LNA]), 2′-o-methyl) where the specificity of a nucleotide is used to detect the microRNA, mRNA, other RNA, or DNA molecule. In these techniques, oligonucleotides may be in solution, on a chip, in a gel or other support. In these techniques, mRNA or microRNA may be detected using fluorescence, or combinations of quenchers and fluorescence, radioactivity, or other chemical or luminescent, methods of detection. In the case where siRNA or therapeutic molecules based on the concepts of RNAi are used, RNA molecules may be modified chemically (for example LNA, 2′-o-methyl), and the measurement of the RNAi therapeutic molecule may be performed by any technique that allows to detect the modification of the RNA/DNA or other molecule that is used as an RNAi therapeutic. 4. In an embodiment of the invention, the measurement of siRNA and/or miRNA and/or target mRNA levels is carried out by qRT-PCR, or by hybridization on microarray or other chip, or by hybridization on gel or membrane, or in solution.

These measurements may for example require a fraction containing RNA from cells, tissues or vesicles, potentially in purified form, isolated by any of several techniques known to the man skilled in the art, such as Trizol extraction.

For example, cells may be cultured in media free of animal serums to avoid contamination with exosomes from said serum. Measurement of RNA from exosomes may require elimination or independence from more abundant cellular RNA, for example by elimination of cells. Advantageously, cells, tissues or vesicles may be cultured, treated, or obtained to preserve or mimic the treatment conditions of a siRNA treatment, or conditions of interest in studying miRNA or siRNA target mRNA. For example, tissues or vesicles used to verify the efficacy of siRNA therapy may be processed at low temperatures immediately and subsequently frozen at temperatures sufficient to preserve the sample's integrity.

For example, to discover mRNA regulated by miR-122 in liver during starvation, liver tissue obtained from starved animals are treated with mimics or inhibitors. Mimics of miRNA or siRNA may retain many or all properties of the endogenous miRNA or its precursors, but may also be modified to enhance its stability or efficacy, as discussed above for miRNA/siRNA molecules.

Inhibitors of miRNA or siRNA may contain one or more perfectly or partially matching target sites for the miRNA or siRNA to be inhibited allowing them to sequester, compete for or cause the degradation of the miRNA or siRNA. These inhibitors may also be modified in similar ways. Some of these modifications may help protect the siRNA-related molecule from degradation, such as a 2′-o-methyl, 2′-o-allyl, 2′-deoxy-fluorouridine modification, or phosphorothioates. Other modifications may also help increase the affinity of the siRNA-related molecule for its target or reduce its off-target effects, such as the locked-nucleic acid modification, in which a methylene bridge connects the 2′-oxygen with the 4′-carbon of the ribose ring. Other modifications may enhance the loading of the correct strand of a siRNA or miRNA into AGO, such as by adding a 5′ phosphate or methyl to one strand of a doublestranded miRNA/miRNA*complex. These types of molecules are now commercially available from several companies such as Qiagen and Ambion of miR-122 may be used, and liver-derived exosomes purified from blood.

By “mRNA targeted by said miRNA and/or by said siRNA”, in the sense of the present invention, is meant any mRNA that may be completely or partially deadenylated or degraded by the administration of siRNA, miRNA or related molecule. Advantageously, its translation may be repressed, or may be cleaved by the siRNA or the miRNA or the related molecule.

Such mRNA targeted by said miRNA and/or by said siRNA may be, for example, miR-196, Lin-28, CAT-1, TNF. MiR-196 is an example of a miRNA that results in cleavage of at least one of its target mRNA, HOXB8 (Yekta et al. Science, 2004, 304: 594 [88]). Lin-28 is an example of an mRNA degraded by a miRNA, miR-125b (Wu et al. Mol. Cell. Biol. 2005, 25: 9198 [89]). CAT-1 is an example of a mRNA subjected to translational repression by a miRNA (Bhattacharyya et al. Cell 2006, 6:1111 [90]). TNF is an example of an mRNA whose translation is activated by miR369 (Vasudevan et al. [74] et [75]). Small RNAs can also regulate transcription in negative and positive ways (Rossi Nat. Chem. Biol. 2007 3: 136 [91]).

In a particular embodiment of the invention, the method of the invention comprises the steps of:

(i) isolating exosomes or vesicles, preferably from a bodily fluid of a patient previously treated with siRNA and/or miRNA,

(ii) measuring, in said exosomes, siRNA and/or miRNA and/or target mRNA levels,

(iii) possibly comparing said levels to a control, then determining the delivery rates and/or efficiency of siRNA and/or miRNA.

By “isolating”, in the sense of the invention, is meant a separation of the exosomes or vesicles from the medium. This separation allows the measurement of siRNA and/or miRNA and/or target mRNA levels. The isolation, or separation, may be accompanied with the purification of the exosomes or vesicles in order to allow the measurement of siRNA and/or miRNA and/or target mRNA levels.

In some embodiments, size exclusion chromatography may be used to isolate the exosomes or vesicles. Size exclusion chromatography techniques are well known in the art. In some embodiments, a void volume of fraction is isolated and comprises the exosomes or vesicles of interest. Further, in some embodiments, the exosomes or vesicles may be further isolated after chromatographic separation by centrifugation techniques (of one or more chromatography fractions), as is well known in the art. In some embodiments, for example, density gradient centrifugation may be used to further isolate the exosomes.

By “levels”, in the sense of the invention, is meant the qualitative (e.g. present or not in the isolated exosome or vesicle) and/or quantitative (e.g. how much is present) measurement of siRNA and/or miRNA and/or target mRNA.

By “control”, in the sense of the present invention, is meant the level of siRNA and/or miRNA and/or mRNA target in exosome of the same individual, but before or after the treatment with iRNA therapeutics, or of exosome of another individual, non treated with iRNA therapeutics, or treated with placebo. The control may be siRNA and/or miRNA and/or mRNA and/or other RNA and/or other molecule permitting/allowing to quantify exosomes or vesicles, or a component thereof of a treated, non-treated, or control treated individual, animal or cells.

By “other RNA”, in the sense of the invention, is meant any RNA molecule other than mRNA molecule. Non-coding RNA such as Xist or BIC, tRNA, rRNA, siRNA, piRNA, miRNA, ribozymes or other RNA molecules in the process of anabolism or catabolism.

In this embodiment of the invention, the control may be carried out by the following of the level of the siRNA previously administered, in the exosomes or vesicles of the target organs or cells. The level of the siRNA in the exosomes or vesicles may be compared with the level of the siRNA in the other compartments of the cell, for example in cytoplasm, or in the extracellular compartment.

The control may also be carried out by the following of the level of the miRNA previously administered, in the exosomes or vesicles of the target organs or cells. The level of the miRNA in the exosomes or vesicles may be compared with the level of the miRNA in the other compartments of the cell, for example in cytoplasm, or in the extracellular compartment.

The control may also be carried out by the following of the level of the mRNA targeted by siRNA and/or miRNA previously administered, in the exosomes or vesicles of the target organs or cells. The level of the target mRNA in the exosomes or vesicles may be compared with the level of the target mRNA in the other compartments of the cell, for example in cytoplasm, or in the extracellular compartment.

The control may also be carried out by the following of the level of another RNA previously administered, in the exosomes or vesicles of the target organs or cells. This other RNA may be any endogenous miRNA (see miRbase [Griffiths-Jones 2008, Nucleic Acids Research 36: (Si) D154) for a list of more than 500 miRNA/species), any synthetic or derived miRNA mimic, siRNA molecule, coding or non-coding RNA. miR-21, miR-141, miR-200a, miR-200b, miR-200c, miR-203, miR-205, miR-214, U6 RNA, Y1 through 6 RNA, tRNA, 28S, 18S or 5S rRNA, 7SK RNA, snoRNA, tubulin mRNA, Glyceraldehyde 3-phosphate dehydrogenase (GAPDH) mRNA, Beta-2-microglobulin mRNA, ubiquitin mRNA, this list not being exhaustive. The level of the other RNA in the exosomes or vesicles may be compared with the level of the other RNA in the other compartments of the cell, for example in cytoplasm, or in the extracellular compartment.

By “other molecule permitting to quantify exosomes or vesicles”, in the sense of the invention, is meant any molecule known as a standard for measuring the level of miRNA, siRNA or RNA in exosomes or vesicles. The target RNA levels may be normalized to a molecule other than an RNA, such as a lipid, protein, or metabolite that is at relatively (variation of +/−35) constant or known levels in exosomes. This may be a molecule of which the level in exosomes or vesicles of untreated or healthy persons is well known by the man skilled in the art. For example, this other molecule may be actin or RRM2 (ribonucleotide reductase M2 polypeptide) mRNA, U6 RNA, CD63, CD82 or other tetraspanin proteins, tsg101, flottilin, EF1a, MHC class I or II, sphingomyelin, cholesterol, GPI-anchored proteins, or phosphatidylethanolamine.

By “component thereof”, in the sense of the invention, is meant any component of exosomes or vesicles. This component may be for example CD63, CD82, PLP or other tetraspanin proteins, tsg101, flottilin, EF1a, MFG-E8, TCTP (translationally controlled tumor protein), MHC class I or II, sphingomyelin, cholesterol, GPI-anchored proteins, or phosphatidylethanolamine.

By “non-treated individual”, in the sense of the invention, is meant an individual having received no administration of siRNA or miRNA or related molecule. This individual may be a healthy person or a patient not yet treated, or treated at a time distant enough (3 days to 2 weeks or longer) that the treatment's effect is reduced with siRNA or miRNA or related molecule.

By “control treated individual”, in the sense of the invention, is meant any individual previously treated with placebo, or some molecule that may not target the expression of the same gene.

In an embodiment of the invention, the bodily fluid may be selected among blood products, urine, lung rinsings, saliva, milk, serum, plasma, ascites, cyst fluid, pleural fluid, peritoneal fluid, cerebral or cerebrospinal fluid, tears, sputum, and other bodily fluids, or the supernatants of cultured cells.

In an embodiment of the invention, the steps (i) to (iii) of the method of the invention are performed before and after siRNA and/or miRNA treatment and/or after a duration (3 days to 2 weeks or longer) that allows the effect of the treatment to be reduced (i.e before and after siRNA and/or miRNA administration).

In other words, the method of the invention is performed twice: once before the treatment of the patient with the siRNA and/or miRNA, and once after the treatment of the patient with the siRNA and/or miRNA, and/or after a duration that allows the effect of the treatment to be reduced (3 days to 2 weeks or longer). It is so possible to compare the levels of siRNA and/or miRNA and/or mRNA targeted in exosomes or vesicles before the treatment and after the treatment. In this embodiment, the control is the measurement of levels of siRNA and/or miRNA in exosomes or vesicles before the treatment.

In other words, the method of the invention comprises the steps of isolating exosomes or vesicles from a person not treated with siRNA and/or miRNA, then measuring, in said exosomes or vesicles, siRNA and/or miRNA and/or target mRNA levels, then possibly comparing said levels with a control, and administering to said person said siRNA and/or miRNA, and then performing the steps (i) to (iii) of the method of the invention, and then determining the delivery rates and/or efficiency of siRNA and/or miRNA therapeutics.

A method according to any of claims 1 to 5, wherein the steps (i) to (iii) are performed before, during and after siRNA and/or miRNA treatment.

By “during siRNA and/or miRNA treatment”, in the sense of the invention, is meant that the method comprises the isolation of exosomes or vesicles from a patient treated with siRNA and/or miRNA, and the measurement, in said exosomes or vesicles, of siRNA and/or miRNA and/or target mRNA levels, at different times after the administration of siRNA and/or miRNA. These different times may be for example 0 minutes, 1 minutes, 2 minutes, 5 minutes, 10 minutes, 30 minutes, 45 minutes, 1 hour, 2 hours, 3 hours, or preferably 1 day to 5 days after delivery of siRNA.

Another object of the invention is a method for determining the efficiency of delivery or activity of a siRNA and/or miRNA to target organs or cells, comprising performing the method for determining the delivery rates and/or efficiency of a siRNA, miRNA or related molecule previously described.

In other words, the method for determining the efficiency of delivery or activity of a siRNA and/or miRNA to target organs or cells, comprises performing the method for determining the delivery rates and/or efficiency of a siRNA, miRNA or related molecule previously described, and the determination of the efficiency of the delivery or of the activity of a siRNA and/or miRNA to target organs or cells.

By “efficiency of delivery of a siRNA and/or miRNA”, in the sense of the invention, is meant the ability, for the siRNA and/or miRNA, to arrive inside cells in a manner that may retain its functional properties. An efficient delivery may be recognized when a change, for example a reduction, in levels of target mRNA occurs in exosomes, compared to the control. An efficient delivery may also be recognized by an accumulation of targeted mRNA on intracellular membranes. An efficient delivery may also be recognized by an enrichment of miRNA or siRNA in exosome.

By “activity of a siRNA and/or miRNA”, in the sense of the invention, is meant the ability of siRNA and/or miRNA to have an effect on the levels of target mRNA and/or protein in the cell. This effect may be a change, for example a reduction in the levels of target mRNA or protein in cell. More particularly, it may be a reduction in the levels of target mRNA or protein in exosomes.

In a particular embodiment of the invention, the efficiency of delivery or activity of said siRNA and/or miRNA is recognized by a reduction or a change of the levels of miRNA, siRNA or target mRNA in exosomes after treatment, i.e. after the administration of miRNA, siRNA or related molecule.

By “reduction”, in the sense of the invention, is meant a diminution of more than 1%, or of more than 10%, or 20%, or 30%, of levels of siRNA or miRNA or mRNA compared to the control.

By “enrichment”, in the sense of the invention, is meant an increase of more than 1%, or of more than 10%, or 20%, or 30%, of levels of siRNA or miRNA or mRNA compared to the control.

In an embodiment of the invention, prior to step i), a step of determining the content of mRNA of whole cell is performed, in order to identify the mRNA target(s).

Advantageously, this step of determining the content of mRNA of whole cell is performed before the administration of siRNA, miRNA or related molecule. Advantageously, it may be performed again after administration of siRNA, miRNA or related molecule.

Advantageously, a reduction or a change of the level of one mRNA indicates that this mRNA is likely the target of the siRNA, miRNA or related molecule.

In an embodiment of the invention, the step of determining the content of mRNA of whole cell is performed using a method selected among mRNA microarray, large-scale method of identifying mRNA or other RNA or DNA targeted by miRNA or siRNA, qRT-PCR, large-scale multigene approach. Optionally, the determination of the content of mRNA of whole cell may be confirmed using a bioinformatic analysis.

In an embodiment of the invention, the method for determining the efficiency of delivery or activity of a siRNA and/or miRNA to target organs or cells comprises the measurement of levels, in membrane fractions and in whole cells of said target organs or cells, of said siRNA and/or said miRNA and/or said mRNA targeted by said miRNA and/or by said siRNA.

By “membrane fraction”, in the sense of the invention, is meant any type of membrane fractions. For example, this may include the fractions enriched in membranes in general, membranes of the endoplasmic reticulum and Golgi, trans-Golgi network, endosomes, lysosomes, autophagosomes, multivesicular bodies or any vesicles that traffic between any of these organelles, or endolysosomal system.

In a particular embodiment of the invention, the measurement of mRNA of whole cells is performed. Ant method for measuring RNA known by the man skilled in the art may be used. Such methods may be qRT-PCR (Quantitative Reverse Transcription polymerase chain reaction) in the many variants in practice (for example SybrGreen, Beacon technologies), or techniques based on the hybridization of oligonucleotide or nucleotide of any length with any variety or combination of modifications (notably locked nucleic acid [LNA]), 2′-o-methyl) where the specificity of a nucleotide is used to detect the RNA. In these techniques, oligonucleotides may be in solution, on a chip, in a gel or other support. In these techniques, RNA may be detected using fluorescence, or combinations of quenchers and fluorescence, radioactivity, or other chemical or luminescent, methods of detection.

Reduced mRNA levels in whole cells in the presence of a miRNA, siRNA or related molecule may validate it as a miRNA target.

Alternatively, the method for determining the efficiency of delivery or activity of a siRNA and/or miRNA to target organs or cells comprises the step of comparing the ratios of mi/siRNA targeted mRNA in exosomes, membrane fractions and whole cells.

In other words, the method of the invention comprises the step of determining the levels of miRNA or siRNA targeted mRNA in exosomes, in membrane fractions and in whole cells, and then calculating the ratio of targeted mRNA in exosomes/targeted mRNA in membrane fractions and the ratio of targeted mRNA in exosomes/targeted mRNA in whole cells.

Alternatively, the method comprises the step of comparing the ratios of miRNA or siRNA (mi/siRNA) targeted mRNA in exosomes vs. cells, or comparing the ratios of mi/siRNA targeted mRNA in exosomes vs. membranes, or comparing the ratios of mi/siRNA targeted mRNA in exosomes vs. cells vs. membranes, or comparing the ratios of mi/siRNA targeted mRNA in membranes vs. cells, or comparing the ratios of mi/siRNA targeted mRNA in exosomes derived from cells containing a mi/siRNA vs. exosomes derived from control cells not containing said mi/siRNA.

In a particular embodiment of the invention, the step (i) of isolating exosomes from a bodily fluid is performed by a technique selected among precipitation, solvent extraction, centrifugation, chromatography, differential centrifugation, size filtration, elimination of whole cells, density separation, electrical separation, or affinity enrichment using characteristic lipid, sugar or protein markers of vesicles.

Optionally, the method further comprises a step of detection, in said exosomes or vesicles, of said siRNA and/or miRNA and/or target mRNA levels, possibly comprising a step of labeling, radiolabeling, fluorescence labeling, qRT-PCR, hybridization, combinations of quenchers and fluorescence, radioactivity, any technique that allows to detect the modification of the RNA/DNA or other molecule.

Another object of the invention is the use of a method according to the invention, to determine mRNA or genes targeted by a miRNA/siRNA or similar molecule, including the determination of “off-target” or undesired effects of said siRNA.

For example, this method may be used to determine a large proportion, for example 10 to 1000, or all, of mRNA targets of a miRNA/siRNA or similar molecule, including the determination of “off-target” or undesired effects of said siRNA.

By “off-target”, in the sense of the invention, is meant the unintended consequences of siRNA-mediated silencing. In other words, an off-target effect may be the regulation of a gene that was not intentionally targeted by the RNAi strategy.

By “determination of undesired effects of siRNA”, in the sense of the invention, is meant the effects observed on RNA or protein that are non desired targets of RNAi. In this case, it is possible that siRNA is used in order to target one, or a few, specific RNA, whereas the effects of siRNA are observed on different, or more RNA or protein. This method may use microarrays or high-throughput sequencing to quantify levels of about 20 000 mRNA at once.

The invention may allow accurate determination of a large proportion of miRNA targets in a cost-effective and efficient manner. We have shown that miRNA and exogenously delivered siRNA are contained in 50-100 nm vesicles secreted by cells called exosomes. We have described in detail the mechanism by which miRNA and a select group of proteins involved in miRNA activity are secreted in exosomes. Using a miRNA reporter system we could follow the trafficking and localization of mRNA targeted by a miRNA compared to a control mRNA. We found that miRNA-targeted mRNA accumulated 5-10-fold on intracellular membranes, and were similarly reduced inside secreted exosomes (FIGS. 1g and 3f). To pursue this finding made with a single mRNA targeted by miRNA we made use of publicly available data that measured the presence of all mRNA in exosomes compared to cells. This dataset confirmed that 87% of known miRNA repressed mRNA are selectively reduced in exosomes. An optimized strategy with further controls, based on opposing enrichment at membranes and in exosomes, and opposing effects when miRNA are overexpressed or inhibited, should dramatically enhance the 87% rate of miRNA target identification, that already greatly exceeds the rates (20-70%) and accuracy of miRNA target identification provided by other available techniques.

mRNA microarrays may be performed on whole cells. Reduced mRNA levels in whole cells in the presence of a miRNA further validate it as a miRNA target (although an unchanged mRNA level in whole cells would not exclude an mRNA as a target).

Bioinformatics confirmation that miRNA targets contain conserved or nonconserved miRNA target sequences may reduce or eliminate secondary or si/miRNA-independent targets.

The combination of the approaches described above may provide a comprehensive si/miRNA target list largely purged of false positives.

Exosome purification, coupled with bioinformatics analysis of microarray or high-throughput sequencing, may be used in order to identify the target(s) of miRNA or siRNA of interest. It is an aspect of the invention to provide a report detailing hundreds, or thousands of targets of a miRNA, and presumably fewer targets of siRNA. Bioinformatics may be used to predict physiological and cellular processes and molecular networks regulated by subgroups of identified miRNA targets. This provides with a comprehensive list of miRNA targets and a concise list of the physiological processes and underlying molecular mechanisms regulated by the given miRNA. The invention may entail monitoring of siRNA delivery and efficacy in vivo. Many methods of siRNA delivery have been demonstrated in animals and are at various stages of testing in humans. These methods include lipid, protein, and viral derived vectors with a large variety of attendant modifications. Sorting of miRNA, mRNA and siRNA contained in a cell into exosomes later released by cells into body fluids may provide an indirect measure of successful RNAi therapy. Exosomes will be purified initially from blood and siRNA or miRNA, and target or control mRNA may be measured by qRT-PCR. If testing has been performed before and after RNAi treatment the efficiency of RNAi may be measured by reductions in the levels of target mRNA in exosomes after treatment.

microRNA targets may be comprehensively and accurately identified by comparing the ratios of mi/siRNA targeted mRNA in exosomes, membrane fractions and whole cells.

Advantageously, the method of the invention may allow to provide a level of confidence in predicted targets that is unknown with techniques of the state of the art. This high level of confidence may be provided by several elements of the method of the invention. First, the differences in mRNA targets enrichment between exosomes and/or cells and/or membranes may provide significant confidence, particularly if all three cellular compartments are compared. Further confidence in the validity of mRNA targets may be gained by using publicly available RNA analysis algorithms available via the internet (e.g. PicTar pictar.mdc-berlin.de/, MIRanda pictar.mdc-berlin. de/, MIRbase microrna.sanger.ac.uk/sequences/, DianaMicroT microrna.gr/ (Maragkakis Nucleic Acids Research 2009 1-4) which analyse may target RNA sequences for the presence of miRNA target sites, the target site accessibility and other parameters. In many variants a score may be assigned that attempts to predict the probability that a given mRNA is targeted by a given miRNA. An independent process resembling these algorithms may be developed to optimize the parameters of searching for miRNA target sites in mRNA in these experiments. An observation that an mRNA level was changed when the level or activity of a miRNA was changed, combined with the presence of at least one more or less conserved or classical miRNA target site (complementary seed region) in the said mRNA may increase the confidence that said miRNA was a true target of the miRNA or siRNA.

Another object of the invention is a method for identifying the target(s) of miRNA or siRNA or inhibitors thereof therapeutics, comprising a method of the invention previsouly described.

In this embodiment, the target of miRNA or siRNA therapeutics is identified by a reduction or a change of the levels of miRNA, siRNA or target mRNA in exosomes after the administration of miRNA, siRNA or related molecule.

Optionally, further assays are carried out to determine whether miRNA targets contain a miRNA binding site, and eventually to determine whether said target site is conserved among the mRNA of several species.

By “conserved among the mRNA of several species”, in the sense of the invention, is meant a sequence present in a similar genomic location in several or more than one species. The determination of mRNA targets of a miRNA may be completed by the analysis of the sequence of potential mRNA targets for miRNA binding sites. For example, if miRNA binding sites are found in the predicted mRNA, there may confer more confidence that a mRNA is really targeted by miRNA. Furthermore, if the miRNA target site is conserved among the mRNA of several species (e.g. flies to humans), this may confer even greater confidence that it is a real miRNA target target. These analyses may be used to reduce the number of predicted miRNA targets in a way not desirable to many users.

A miRNA or siRNA is generally about 20 nucleotides long, but it advantageously only may require exact or close to exact sequence-specific matching with a target RNA along nucleotide 2-7 of the miRNA/siRNA to effectively reduce the expression of the target protein. Computer algorithms (e.g. PicTar pictar.mdc-berlin.de/, MIRanda pictar.mdc-berlin.de/, MIRbase microrna.sanger.ac.uk/sequences/, DianaMicroT microrna.gr/ (Maragkakis Nucleic Acids Research 2009 1-4) have been developed to predict, bio-informatically, the RNA targets of miRNA/siRNA, however since our prediction rules have many exceptions, these computer algorithms are imprecise and prone to high errors of false positives or false negatives. However, the use of the invention without subsequent bio-informatic analysis of predicted targets may give a potentially very complete list of RNA targeted by the miRNA, many of which may not be predicted by computer algorithms or other methods. Advantageously, a more manageable list of RNA targets of a miRNA/siRNA the aforementioned computer algorithms may be used to retain only those RNA with miRNA target sites defined at different stringencies (depending on the length of target RNA list the user desired). The resulting list, after the computer algorithms, may provide higher confidence that each target is a true target, but may provide a potentially less complete list of all targets. The computer algorithms may score the probability that a RNA is a target of a miRNA/siRNA by length and placement (in miRNA, in mRNA) of sequence matching, by accessibility of this site in the mRNA to miRNA and associated proteins (by analyzing RNA folding energies of the region) (Maragkakis Nucleic Acids Research 2009 1-4 [92]).

A method for determining the efficiency of a treatment with siRNA and/or miRNA therapeutics or other molecule, comprising a method according to the invention, as previously described.

Advantageously, the treatment may performed with proteins, lipids, RNA or other molecules involved in the formation, interactions, trafficking of molecules or vesicles to or from, or activities of the multivesicular body (MVB) for modulating the activity and/or the cell-to-cell transfer of RNA, small RNA, for example miRNA, siRNA and piRNA, mRNA or non-coding RNA.

Advantageously, the molecules may be issued from the endosomal sorting complex required for transport (ESCRT), Alix, LBPA (lysobisphosphatidic acid), or from the formation of lipid rafts, metabolism or sorting of cholesterol (e.g. NPC1, HMGCR, HMG CoA Reducatse) or sphingomyelin (sphingomyelinase), for example GW4869.

In this method, the proteins and small RNA, such as miRNA, siRNA and piRNA, may be included in vesicles secreted by the multivesicular body (MVB) or other mechanisms in the extracellular space.

Advantageously, the vesicles may be exosomes, or exosome-like vesicles.

In a particular embodiment of the invention, the proteins may be selected from the group consisting of Alix, Hrs, vps36 (Vacuolar protein sorting associated protein 36), EAP30 (ELL-associated protein of 30 kDA, SNF8), EF1a (elongation factor 1a) and BIG2, hps4 (Hermansky-Pudlak syndrome 4), hps 1 (Hermansky-Pudlak syndrome 1), PRNP (prion protein), SCF ubiquitin ligase (Skp1-Cullin-F-box protein), Surfeit-4, V0 or V1 ATPase (V0 or V1 adenosine triphosphatase), vps41, COGC4 (Conserved Oligomeric Golgi Component 4), ATG3 (autophagy-related protein 3), ATG8, COG4, PI3K (Phosphatidylinositol 3-Kinase), NEDD4L (neural precursor cell expressed, developmentally down-regulated 4-like), ARFGEF4 (ADP-ribosylation factor Guanine nucleotide exchange factor-4 protein), CHML (Choroideremia-lik), RAB10 (ras-related GTP-binding protein 10), RAB35, RALB (V-ral simian leukemia viral oncogene homolog B), RAPGEF6 (Rap Guanine Nucleotide exchange factor 6), SCD (stearoyl-CoA desaturase), GIPC1 (GIPC PDZ domain containing family, member 1), SCGB1D1 (secretoglobin, family 1D, member 1), UBE2M (Ubiquitin-conjugating enzyme E2M), USP10 (ubiquitin specific protease 10), EEF2 (eukaryotic translation elongation factor 2), LILRB1 (leukocyte immunoglobulin-like receptor, subfamily B), RAB36, RANBP2 (Ran Binding Protein 2), SFRP2 (secreted frizzled-related protein 2), SLC4A4 (Solute carrier family 4, sodium bicarbonate cotransporter, member 4), SMPD3 (sphingomyelin phosphodiesterase 3), Sphingomyelinase, sphingomyelin synthase 1, sphingomyelin synthase 2, Epopamil binding protein, usp22 (Ubiquitin-specific protease 22), trpc3 (Transient receptor potential cation channel, subfamily C, member 3), CLCN7 (Chloride channel 7), CTSC (cathepsin C), LAMR1 (Laminin receptor 1), RNF32 (Ring finger protein 32), ERI3 (Enhanced RNAi-3), HMGCR (3-hydroxy-3-methylglutaryl CoA reductase), NPC1 (Niemann-Pick disease, type C1), SLC6A4 (sodium-dependent serotonin transporter, solute carrier family 6 member 4), FAU, THEA (ACOT11, acyl-coenzyme A thioesterase 11), CKAP4, COG1-8 proteins, vps1-45 proteins, CHMP family proteins, sorting nexins, rab 5, 7, 9, 38, Arf2, Arf6, GGA1-3, sphingomyelin and sterol metabolism genes and drugs (e.g. GW4869, sphingomyelin esterase), drugs and genes affecting cholesterol or lipid raft partitioning and metabolism in relation to their involvement of sorting into MVB or exosomes, notably NPC1, HMGCR, and the statin classes of cholesterol lowering drugs (e.g. mevastatin).

Advantageously, this method may be used for the prevention or treatment of diseases selected from the group comprising mycobacteria and other intracellular pathogens, neurodegenerative diseases (for example Alzheimer disease, Huntington disease, fragile X syndrome, prion diseases such as Creutzfeld-Jacob, Parkinson's disease), Hermansky-Pudlak syndromes, Niemann Pick Disease or other conditions affecting cholesterol levels including cardiovascular disease, diseases generated by HTLV-1 (Human T-lymphotropic virus-1) and HTLV-2, HIV-1, other retroviruses, or viruses or other pathogens producing miRNA, for example KSHV (Kaposi's sarcoma-associated herpesvirus), and EBNA (Epstein Barr Nuclear Antigen), cancers, developmental deficiencies, and viral infections.

Advantageously, the invention may allow to recognize which part of a disease may be due to dysregulation of miRNA or other small RNA. Many diseases, such as Creutzfeld-Jacob, Alzheimer's, AIDS and others previously listed may affect endolysosomal, and sometimes more specifically MVB-exosome processes. The invention may allow to see that some of the symptoms of these diseases may be due to effects on miRNA or small RNA pathways. The invention may allow to see that, by affecting MVB and exosomes, Alzheimer's disease may detrimentally affect miRNA activity.

Altering or reinstating the miRNA pathway may be a treatment for Alzheimer's disease. In such a case, miRNA activity may be augmented or re-instated by delivering various components of miRNA activity (e.g. miRNA, Dicer, GW182, AGO) to cells or targeting the endolysosomal system to secondarily alter miRNA activity.

Therefore, the invention may allow to see the need to treat disease symptoms by increasing or altering miRNA or other small RNA activity by any number of means known or yet unknown.

Indeed, the method of the invention may be used, after determining the efficiency of a treatment with siRNA and/or miRNA therapeutics or other molecule, to select the better treatment or dose of treatment of a disease, and so to use this treatment to treat or prevent the disease.

Another object of the invention is a method for genotyping and/or characterizing the condition of a person, a tumor or a fetus, comprising a method according to the invention.

Another object of the invention is a method for controlling the activity of miRNA or small RNA in an organism, a cell or a plant, comprising the genetic modification of said organism or the administration in the organism of a protein, or a chemical that modify the activity of this protein, or a siRNA or miRNA or molecule related thereof targeting this protein, this protein being selected among Alix, Hrs, vps36 (Vacuolar protein sorting associated protein 36), EAP30 (ELL-associated protein of 30 kDA, SNF8), EF1a (elongation factor 1a) and BIG2, hps4 (Hermansky-Pudlak syndrome 4), hps 1 (Hermansky-Pudlak syndrome 1), PRNP (prion protein), SCF ubiquitin ligase (Skp1-Cullin-F-box protein), Surfeit-4, V0 or V1 ATPase (V0 or V1 adenosine triphosphatase), vps41, COGC4 (Conserved Oligomeric Golgi Component 4), ATG3 (autophagy-related protein 3), ATG8, COG4, PI3K (Phosphatidylinositol 3-Kinase), NEDD4L (neural precursor cell expressed, developmentally down-regulated 4-like), ARFGEF4 (ADP-ribosylation factor Guanine nucleotide exchange factor-4 protein), CHML (Choroideremia-like), RAB10 (ras-related GTP-binding protein 10), RAB35, RALB (V-ral simian leukemia viral oncogene homolog B), RAPGEF6 (Rap Guanine Nucleotide exchange factor 6), SCD (stearoyl-CoA desaturase), GIPC1 (GIPC PDZ domain containing family, member 1), SCGB1D1 (secretoglobin, family 1D, member 1), UBE2M (Ubiquitin-conjugating enzyme E2M), USP10 (ubiquitin specific protease 10), EEF2 (eukaryotic translation elongation factor 2), LILRB1 (leukocyte immunoglobulin-like receptor, subfamily B), RAB36, RANBP2 (Ran Binding Protein 2), SFRP2 (secreted frizzled-related protein 2), SLC4A4 (Solute carrier family 4, sodium bicarbonate cotransporter, member 4), SMPD3 (sphingomyelin phosphodiesterase 3), Sphingomyelinase, sphingomyelin synthase 1, sphingomyelin synthase 2, Epopamil binding protein, usp22 (Ubiquitin-specific protease 22), trpc3 (Transient receptor potential cation channel, subfamily C, member 3), CLCN7 (Chloride channel 7), CTSC (cathepsin C), LAMR1 (Laminin receptor 1), RNF32 (Ring finger protein 32), ERI3 (Enhanced RNAi-3), HMGCR (3-hydroxy-3-methylglutaryl CoA reductase), NPC1 (Niemann-Pick disease, type C1), SLC6A4 (sodium-dependent serotonin transporter, solute carrier family 6 member 4), FAU ( ) THEA (ACOT11, acyl-coenzyme A thioesterase 11).

Advantageously, the ESCRT genes, for example tsg101 and vps45, affect miRNA activity in plants. The inventors surprisingly demonstrate that plants with TDNA insertions in the plant homologues of tsg101 and vps45 (transposon inserted in and disrupting expression of the gene) may show accumulation of proteins regulated by miRNA, but not control proteins. Therefore, targeting the same groups of genes, proteins and lipids as listed for MVB may be used to modulate anti-pathogen defense of plants, for example anti-viral defense, non-cell autonomous RNA silencing, maintenance of gene expression in genetically modified plants, or the epigenetic maintenance of plant traits (by affecting germ-line re-setting of heterochromatin).

Advantageously, it may be possible to use exosomes from any bodily fluid to characterize the levels of miRNA and probably other small RNA of an individual and thereby ascertain their relative state of health, since the inventors demonstrate that exosomes contain the same miRNA as the cells from which they derive, and in proportionally similar quantities. Exosomes and cells may contain the same miRNA (e.g. both contain miR-16, miR-27a, miR206), and the relative quantities of each miRNA (e.g. the profile) may be the same in the cells and exosomes (e.g. in both cells and exosomes mIR-16 is most abundant, there is five-fold less miR-206, three-fold less miR-27a). Since the profile of miRNA from cells may be used to give a diagnosis or prognosis, the profile of miRNA in exosomes may be used for diagnosis or prognosis as well.

For example, Let-7a is downregulated in prostrate cancer (Spizzo 2009 Cell 137: 586, [96]). If prostrate cancer can be diagnosed by lower levels of let-7a in the cancer cells, exosomes may be used, from a source likely to contain exosomes from the cancerous tissue (e.g. urine for prostrate), to evaluate relative levels of let-7a and arrive at a diagnosis and/or prognosis of cancer without performing a biopsy. At the same time, the profile of miRNA obtained in fluid deriving from a site of a possible tumor (observed on x-ray for example) may be used to determine the type of cell that was cancerous, by the profile or pattern of miRNAs expressed, and thereby aid in the establishment of the prognosis. Additionally, detection of specific miRNA or other small RNA, like piRNA, associated with a given state, such as transformed or pluripotent cells, may help in determining the severity of a patient's state, such as cancer. In another example, using exosomes from amniocentric fluid, sex-specific miRNA may be used to determine a baby's sex a few days after conception.

Another object of the invention is a method of diagnostic or prognostic of disease based on the use of mRNA in exosomes where this is differentially present in exosomes due to differential splicing or gene regulation in the cell that is dependent upon small RNA.

The analysis may be performed by enriching exosomes or vesicles from given bodily fluid by any step known by the man skilled in the art, for example exclusion-limit filtration, differential ultracentrifugation, antibody-bead based purification using markers specific for exosomes or vesicles. RNA may be enriched by methods such as Trizol for analysis of miRNA or other small RNA or mRNA by qRT-PCR, microarray or any other method allowing to establish relative quantities of small RNA molecules.

The change in the amounts of miRNA or mRNA may be evaluated in relation to the amounts of other RNA, protein, lipid, or other molecule in exosomes. Advantageously it may be possible to compare these amounts to those of control treated individuals, for example before and after symptoms or treatment.

Another object of the invention is the use of a method according to the invention, for the screening of candidate molecules for diagnosis or treatment.

Another object of the invention is a method to identify diagnostic or prognostic markers such as dysregulated miRNA or mRNA in a disease or pathological condition with the aim of establishing diagnostic or prognostic criteria. Advantageously, exosomes may be used to evaluate the dysregulation of miRNA as they are less difficult to obtain from patients compared to other part of cells. Advantageously, exosomes may be used to evaluate the dysregulation of miRNA by various methods, for example microarray or qRT-PCR. mRNA markers of disease may be selected from those already known to be diagnostic or prognostic of disease (e.g. Spizzo 2009 Cell 137: 586 [96]), or may be newly developed using the invention or other techniques. The steps to detect these markers may be the same as described above, isolation of exosomes, detection of specific miRNA by various methods.

Alternatively, mRNA microarrays on exosome RNA may be used to determine mRNA up- and down-regulated in a disease condition. Bio-informatics could be subsequently used to predict miRNA(s) linked to the disease.

The candidate molecules, markers or therapeutic delivery agents (such as liposomes) or delivery methods may be selected if a reduction or a change in the levels of miRNA, siRNA or target mRNA in exosomes after the administration of miRNA, siRNA or related molecule is observed.

Advantageously, the candidate molecules may be selected among proteins or lipids involved in the formation of the multivesicular body (MVB) for modulating the activity and/or the cell-to-cell transfer of RNA, small RNA, for example miRNA, siRNA and piRNA, mRNA or non-coding RNA.

More precisely, the proteins may be selected from the group consisting of Alix, Hrs, vps36, EAP30, EF1a, BIG2, hps4, PRNP, SCF ubiquitin ligase, Surfeit-4, V0 or V1 ATPase, vps41, N-sphingomyelinase.

Advantageously, the method of the invention may be used for the prevention or treatment or diagnosis of diseases selected from the group comprising tuberculosis, neurodegenerative diseases (for example Alzheimer disease, Huntington disease, fragile X syndrome), diseases generated by HTLV-1 and -2, HIV-1, viruses producing miRNA (for example KSHV, EBNA) or prions, cancers, developmental deficiencies, and viral or other infections.

Another object of the invention is a kit comprising:

(a) means for isolating exosomes or vesicles from a bodily fluid,

(b) means for measuring, in said exosomes, siRNA and/or miRNA and/or target mRNA levels.

Advantageously, the kit of the invention may further comprise means of comparison with a control.

For the part of the invention concerning identifying all targets of a miRNA/siRNA, controls may refer to cells or animals treated with a similar but ineffective molecule. In one embodiment of the invention, it may be only necessary to measure mRNA quantity vs. the amount of vesicles, or a component thereof. In such embodiment, the control would be the amount of vesicles, or a component thereof. In other cases the control may be an untreated individual etc.

Another object of the invention is the use of proteins or lipids involved in the formation of the endolysosomal system for modulating the activity and/or the cell-to-cell transfer of RNA, small RNA, for example miRNA, siRNA and piRNA, mRNA or non-coding RNA.

By “modulating the activity of RNA, small RNA, for example miRNA, siRNA and piRNA, mRNA or non-coding RNA”, in the sense of the invention, is meant any induction of a modification in the RNA or small RNA synthesis in a cell, or in the level of the RNA or small RNA in a cell, or in its ability to modulate the transcription of a RNA, induce RNA decapping, deadenylation or degradation, modulate translation or otherwise inhibit the expression of a protein. Advantageously, the modification may be a reduction, or an inhibition of genes, compared with the activity level of RNA, small RNA, for example miRNA, siRNA and piRNA, mRNA or non-coding RNA before the use or said proteins or lipids.

By “modulating the cell-to-cell transfer of RNA, small RNA, for example miRNA, siRNA and piRNA, mRNA or non-coding RNA”, in the sense of the invention, is meant a modification in the transfer of RNA, small RNA, for example miRNA, siRNA and piRNA, mRNA or non-coding RNA from a cell to another cell, compared with the level of transfer before the use or said proteins or lipids. Notably, this may also include the ability to produce exosomes or vesicles from cells, for example in culture media, then transfer the exosomes to an individual to mediate RNA transfer into cells of the individual.

Compared to the state of the art, the inventors surprisingly found that the inhibition of genes according to the present invention has minor effects on cells, such as minor defects in cellular division.

Advantageously, the proteins may be issued from the endosomal sorting complex required for transport (ESCRT).

Advantageously, these proteins and small RNA, such as miRNA, siRNA and piRNA, may be included in vesicles secreted by the multivesicular body (MVB) in the extracellular space.

In one embodiment of the invention, the vesicles may be exosomes.

In another embodiment of the invention, the proteins may be selected from the group consisting of Alix, Hrs, vps36, EAP30, EF1a and BIG2, hps4, PRNP, SCF ubiquitin ligase, Surfeit-4, V0 or V1 ATPase, vps41, or any protein encoded by a gene identified by the method of claim 36 hps4, PRNP, SCF ubiquitin ligase, Surfeit-4, V0 or V1 ATPase, vps41, COGC4, ATG3, ATG8, COG4, PI3K, NEDD4L, ARFGEF4, CHML, RAB10, RAB35, RALB, RAFGEF6, SCD, GIPC1, SCGB1D1, UBE2M, USP10, EEF2, LILRB1, RAB36, RANBP2, SFRP2, SLC4A4, SMPD3, Sphingomyelinase, Epopamil binding protein, usp22 (ubiquitin specific peptidase 22, trpc3, CLCN7, CTSC, LAMR1, RNF32, PRNIP, HMGCR, NPC1, SLC6A4, FAU, THEA, ZP2, SCGB1D1.

In a particular aspect of the invention, the proteins or lipids involved may be used for the prevention or treatment of diseases selected from the group comprising tuberculosis, neurodegenerative diseases (for example Alzheimer disease, Huntington disease, fragile X syndrome), diseases generated by HTLV-1 and -2, HIV-1, viruses producing miRNA (for example KSHV, EBNA) or prions, cancers, developmental deficiencies, and viral infection.

In another particular aspect of the invention, the proteins or lipids may be used for genotyping and/or characterizing the condition of a person, a tumor or a fetus.

Another object of the invention is the use of a protein selected from the group consisting of Alix, Hrs, vps36, EAP30, EF1a and BIG2, hps4, PRNP, SCF ubiquitin ligase, Surfeit-4, V0 or V1 ATPase, vps41, for targeting a body of the endolysosomal system, for the treatment of diseases selected from the group comprising tuberculosis, neurodegenerative diseases (for example Alzheimer disease, Huntington disease, fragile X syndrome), diseases generated by HTLV-1 and -2, HIV-1, viruses producing miRNA (for example KSHV, EBNA) or prions, cancers, developmental deficiencies, and viral infections.

In other words, another object of the invention is a method for the treatment of diseases selected from the group comprising tuberculosis, neurodegenerative diseases (for example Alzheimer disease, Huntington disease, fragile X syndrome), diseases generated by HTLV-1 and -2, HIV-1, viruses producing miRNA (for example KSHV, EBNA) or prions, cancers, developmental deficiencies, and viral infections comprising administering to a subject in need thereof a pharmaceutically effective amount of a protein or above selected from the group consisting of Alix, Hrs, vps36, EAP30, EF1a and BIG2, hps4, PRNP, SCF ubiquitin ligase, Surfeit-4, V0 or V1 ATPase, vps41, for targeting a body of the endolysosomal system.

In still other words, another object of the invention is the use of a protein selected from the group consisting of Alix, Hrs, vps36, EAP30, EF1a and BIG2, hps4, PRNP, SCF ubiquitin ligase, Surfeit-4, V0 or V1 ATPase, vps41, Alix, Hrs, vps36 (Vacuolar protein sorting associated protein 36), EAP30 (ELL-associated protein of 30 kDA, SNF8), EF1a (elongation factor 1a) and BIG2, hps4 (Hermansky-Pudlak syndrome 4), hps 1 (Hermansky-Pudlak syndrome 1), PRNP (prion protein), SCF ubiquitin ligase (Skp1-Cullin-F-box protein), Surfeit-4, V0 or V1 ATPase (V0 or V1 adenosine triphosphatase), vps41, COGC4 (Conserved Oligomeric Golgi Component 4), ATG3 (autophagy-related protein 3), ATG8, COG4, PI3K (Phosphatidylinositol 3-Kinase), NEDD4L (neural precursor cell expressed, developmentally down-regulated 4-like), ARFGEF4 (ADP-ribosylation factor Guanine nucleotide exchange factor-4 protein), CHML (Choroideremia-lik), RAB10 (ras-related GTP-binding protein 10), RAB35, RALB (V-rat simian leukemia viral oncogene homolog B), RAPGEF6 (Rap Guanine Nucleotide exchange factor 6), SCD (stearoyl-CoA desaturase), GIPC1 (GIPC PDZ domain containing family, member 1), SCGB1D1 (secretoglobin, family 1D, member 1), UBE2M (Ubiquitin-conjugating enzyme E2M), USP10 (ubiquitin specific protease 10), EEF2 (eukaryotic translation elongation factor 2), LILRB1 (leukocyte immunoglobulin-like receptor, subfamily B), RAB36, RANBP2 (Ran Binding Protein 2), SFRP2 (secreted frizzled-related protein 2), SLC4A4 (Solute carrier family 4, sodium bicarbonate cotransporter, member 4), SMPD3 (sphingomyelin phosphodiesterase 3), Sphingomyelinase, sphingomyelin synthase 1, sphingomyelin synthase 2, Epopamil binding protein, usp22 (Ubiquitin-specific protease 22), trpc3 (Transient receptor potential cation channel, subfamily C, member 3), CLCN7 (Chloride channel 7), CTSC (cathepsin C), LAMR1 (Laminin receptor 1), RNF32 (Ring finger protein 32), ERI3 (Enhanced RNAi-3), HMGCR (3-hydroxy-3-methylglutaryl CoA reductase), NPC1 (Niemann-Pick disease, type C1), SLC6A4 (sodium-dependent serotonin transporter, solute carrier family 6 member 4), FAU, THEA (ACOT11, acyl-coenzyme A thioesterase 11), CKAP4, COG1-8 proteins, vps1-45 proteins, CHMP family proteins, sorting nexins, rab 5, 7, 9, 38, Arf2, Arf6, GGA1-3, sphingomyelin and sterol metabolism genes and drugs (e.g. GW4869, sphingomyelin esterase), drugs and genes affecting cholesterol or lipid raft partitioning and metabolism in relation to their involvement of sorting into MVB or exosomes, notably NPC1, HMGCR, and the statin classes of cholesterol lowering drugs (e.g. mevastatin) for targeting a body of the endolysosomal system, for the manufacture of a therapeutic for the treatment of diseases selected from the group comprising tuberculosis, neurodegenerative diseases (for example Alzheimer disease, Huntington disease, fragile X syndrome), diseases generated by HTLV-1 and -2, HIV-1, viruses producing miRNA (for example KSHV, EBNA) or prions, cancers, developmental deficiencies, and viral infections.

Another object of the invention is the use of a siRNA or miRNA or molecule related thereof targeting a protein selected from the group consisting of Alix, Hrs, vps36, EAP30, EF1a and BIG2, hps4, PRNP, SCF ubiquitin ligase, Surfeit-4, V0 or V1 ATPase, vps41, for targeting a body of the endolysosomal system, for the treatment of diseases selected from the group comprising tuberculosis, neurodegenerative diseases (for example Alzheimer disease, Huntington disease, fragile X syndrome), diseases generated by HTLV-1 and -2, HIV-1, viruses producing miRNA (for example KSHV, EBNA) or prions, cancers, developmental deficiencies, and viral infections.

Another object of the invention is a method of treatment of a disease selected from the group comprising tuberculosis, neurodegenerative diseases (for example Alzheimer disease, Huntington disease, fragile X syndrome), diseases generated by HTLV-1 and -2, HIV-1, viruses producing miRNA (for example KSHV, EBNA) or prions, cancers, developmental deficiencies, and viral infections, comprises the administration to an individual in need thereof, of a therapeutically effective amount of a siRNA or miRNA or molecule related thereof targeting a protein selected from the group consisting of Alix, Hrs, vps36, EAP30, EF1a and BIG2, hps4, PRNP, SCF ubiquitin ligase, Surfeit-4, V0 or V1 ATPase, vps41, for targeting a body of the endolysosomal system.

Another object of the invention is a method of treatment of a disease selected from the group comprising tuberculosis, neurodegenerative diseases (for example Alzheimer disease, Huntington disease, fragile X syndrome), diseases generated by HTLV-1 and -2, HIV-1, viruses producing miRNA (for example KSHV, EBNA) or prions, cancers, developmental deficiencies, and viral infections, comprises the administration to an individual in need thereof, of a therapeutically effective amount of a chemical that modify the activity of a protein selected from the group consisting of Alix, Hrs, vps36, EAP30, EF1a and BIG2, hps4, PRNP, SCF ubiquitin ligase, Surfeit-4, V0 or V1 ATPase, vps41, Alix, Hrs, vps36 (Vacuolar protein sorting associated protein 36), EAP30 (ELL-associated protein of 30 kDA, SNF8), EF1a (elongation factor 1a) and BIG2, hps4 (Hermansky-Pudlak syndrome 4), hps 1 (Hermansky-Pudlak syndrome 1), PRNP (prion protein), SCF ubiquitin ligase (Skp1-Cullin-F-box protein), Surfeit-4, V0 or V1 ATPase (V0 or V1 adenosine triphosphatase), vps41, COGC4 (Conserved Oligomeric Golgi Component 4), ATG3 (autophagy-related protein 3), ATG8, COG4, PI3K (Phosphatidylinositol 3-Kinase), NEDD4L (neural precursor cell expressed, developmentally down-regulated 4-like), ARFGEF4 (ADP-ribosylation factor Guanine nucleotide exchange factor-4 protein), CHML (Choroideremia-lik), RAB10 (ras-related GTP-binding protein 10), RAB35, RALB (V-ral simian leukemia viral oncogene homolog B), RAPGEF6 (Rap Guanine Nucleotide exchange factor 6), SCD (stearoyl-CoA desaturase), GIPC1 (GIPC PDZ domain containing family, member 1), SCGB1D1 (secretoglobin, family 1D, member 1), UBE2M (Ubiquitin-conjugating enzyme E2M), USP10 (ubiquitin specific protease 10), EEF2 (eukaryotic translation elongation factor 2), LILRB1 (leukocyte immunoglobulin-like receptor, subfamily B), RAB36, RANBP2 (Ran Binding Protein 2), SFRP2 (secreted frizzled-related protein 2), SLC4A4 (Solute carrier family 4, sodium bicarbonate cotransporter, member 4), SMPD3 (sphingomyelin phosphodiesterase 3), Sphingomyelinase, sphingomyelin synthase 1, sphingomyelin synthase 2, Epopamil binding protein, usp22 (Ubiquitin-specific protease 22), trpc3 (Transient receptor potential cation channel, subfamily C, member 3), CLCN7 (Chloride channel 7), CTSC (cathepsin C), LAMR1 (Laminin receptor 1), RNF32 (Ring finger protein 32), ERI3 (Enhanced RNAi-3), HMGCR (3-hydroxy-3-methylglutaryl CoA reductase), NPC1 (Niemann-Pick disease, type C1), SLC6A4 (sodium-dependent serotonin transporter, solute carrier family 6 member 4), FAU, THEA (ACOT11, acyl-coenzyme A thioesterase 11), CKAP4, COG1-8 proteins, vps1-45 proteins, CHMP family proteins, sorting nexins, rab 5, 7, 9, 38, Arf2, Arf6, GGA1-3, sphingomyelin and sterol metabolism genes and drugs (e.g. GW4869, sphingomyelin esterase), drugs and genes affecting cholesterol or lipid raft partitioning and metabolism in relation to their involvement of sorting into MVB or exosomes, notably NPC1, HMGCR, and the statin classes of cholesterol lowering drugs (e.g. mevastatin), for targeting a body of the endolysosomal system.

By “modify”, in the sense of the invention, is meant a reduction or an increase of the activity of the protein.

The present invention so finds many applications, some of them are described hereafter.

Generally speaking, RNAi delivery is the major barrier to treatment. The invention provide a cost-effective measure of RNAi delivery in each patient to help optimize delivery methods in early clinical testing, enhance outcome measures of late stage clinical trials and chance of approval by drug regulatory bodies, and allow personalized dosing and drug selection.

The invention may allow to identify which genes really do affect miRNA activity, as the studies show the ability of the endolysosomal system to control small RNA activity. These genes may include some that may be more directly involved in miRNA activity than the ESCRT complex, for example genes involved in Golgi vesicle sorting, multivesicular body-lysosome sorting, this list not being exhaustive. Our work suggests the ESCRT complex affects miRNA activity, but in fact the effect of the ESCRT complex may be an indirect effect on other multivesicular body processes, such as cholesterol/lipid-raft sorting, BLOC complexes (Biogenesis of Lysosome-related Organelles Complexes), ceramide, sphingomyelin, GGA complexes (Golgi-localised, γ-ear containing, ADP-ribosylation factor-binding protein) or other things.

The present invention may allow to deliver active siRNA and miRNA in cells, by means which allows to target a specific cellular type, and may allow to transfer siRNA in the entire body by blood, or lymphatic vessels or diffusion in intercellular space and fluids.

The present invention may also allow to increase or reduce the activity of siRNA and miRNA, or to extend the effect thereof, either by cell-to-cell transfer, compartmentalizing or controlling the components of the small RNA pathway, or by regulating DNA transcription by heterochromatin. Many viruses and diseases, such as HIV-1 or prions that associate with the MVB or miRNA, may thus be targeted by the present invention. Furthermore the present invention may provide a way to diagnose the condition (e.g. tumor) or genotype of a person (notably of a fetus with blood from a mother) or a tumor by a blood sample.

The present invention may provide new means to selectively control the activity of subsets of small RNA, and effective means to inhibit the proliferation of pathogens.

The present invention may also allow for example to genotype a fetus, to evaluate a cancer with a blood sample resulting in less risks than the techniques used at present. The present invention may allow to transfer siRNA, miRNA or other small RNA into a cell by an endogenous means (minimal toxic and immunological effects) which can be targeted to a specific cellular type by means well known in the art.

The present invention may have medical applications as to modulate the siRNA or miRNA activity, and activity of other types of small RNA used for the treatment of diseases or a modulation of treatments in any organism.

The invention may also have applications in miRNA regulating for diseases wherein small RNA, their processing or effects are important, for example cancers, developmental problems, embryogenesis, fertilization and viral infections.

The invention may also have applications in the delivery of any kind of RNA, and notably small RNA or mRNA, to target cells.

Another potential application of the invention is the capacity to carry out an analysis of RNA, miRNA or potentially DNA included in blood exosomes from blood or another source or other components from a person or a mother. It may allow to genotype or characterize otherwise the condition of a person, a tumor or a foetus in a less dangerous manner than the techniques used at present (e.g. obtaining a sample of amniotic fluid or biopsy). Many neurodegenerative diseases, viral diseases or diseases based on prions depend on RNA during infection and on the same proteins as those involved in the miRNA machinery. Treatments for these diseases may derive from the invention. Furthermore, the invention may allow the treatment of autoimmune diseases, in which antibodies that bind proteins associated with miRNA are present, and for many diseases for which the MVB or miRNA are important, for example tuberculosis, HTLV-1 or -2, and HIV-1, viruses producing miRNA (KSHV, EBNA, etc. . . . ), prions, or any infectious agent targeting miRNA or MVB pathways, and other neurodegenerative diseases as Alzheimer disease, Huntington disease or fragile X syndrome.

Another object of the invention is a method to measure the efficiency or activity of endogenously produced small RNA, such as miRNA. The activity/efficiency or expression/absence of endogenously produced miRNA or small RNA may be measured by measuring the small RNA directly in the exosome, or measuring its target RNA, usually a mRNA. This may be the basis for diagnostic or prognostic tests based on the activity or presence or efficiency of small RNA produced by the cell itself.

### BRIEF DESCRIPTION OF THE DRAWINGS

FIG. 1: Confocal micrograph showing Ago2 and GW182 co-localize with the multivesicular body. Cells were loaded with N-Rh-PE to label the MVB one day after transfection with the given plasmid and examined by confocal microscopy. Cells were transfected with (A) CD82-YFP, (B) Sec61β, (C) eGFPN1, (D, E) GFP-GW182, or (F,G) GFP-Ago2. In (H) cells were not loaded with N-Rh-PE. Instead cells were co-transfected with plasmids expressing Gag-RFP and Ago2-GFP. In (I) cells transfected with Ago2-GFP were lightly fixed, permeabilized and Dcp1a was detected by immunofluorescence.

FIG. 2: Confocal micrograph showing the localization of GFP-GW182 to the MVB is independent of stress granule formation. Cells were co-transfected with plasmids expressing GFP-GW182 and (A) TIA-1 dominant negative or (B) a constitutively active eIF2a S51D, that respectively inhibit or drive the formation of stress granules. In (C) cells expressing GFP-GW182 and loaded with N-Rh-PE (N-dioleoylphosphatidylethanolamine) were treated with cycloheximide to dissolve P-bodies and stress granules.

FIG. 3: Westernblot showing GW182, but not Dcp1a (mRNA-decapping enzyme 1A), is ubiquitinated. Co-immunoprecipitations of (A) transferring receptor, (B) Dcp1a, or (C) GW182 and Ub were performed. Proteins were detected with antibodies recognizing the endogenous protein. Controls consist of species and isotype matched (when possible) antibody immunoprecipitations. In the right panel of (C) immunoprecipitated ubiquitinated proteins were blotted with antibody recognizing Ge-1.

FIG. 4: Members of the ESCRT complex affect miRNA activity but not localization of GW182 to the MVB. (A) Confocal micrography showing cells loaded with N-Rh-PE to label the MVB one day after transfection with GFP-GW182 and 50 nM of siRNA targeting Alix or vps36. (B) Shows the percentage of expression of Let-7a vs. mutated site (Normalized Renilla Luciferase). Knockdown of some members of the ESCRT complex inhibits activity of Let-7a. Analysis of the levels of Let-7a vs mutated site expression for the control, tsg101, vps36, hrs, alix, and GW 182. Cells were transfected with plasmids expressing Renilla luciferase attached to an intact or mutated Let-7a target site, 50 nM siRNA, and pGL3 expressing firefly luciferase (n=6). (C) Knockdown of some members of the ESCRT complex inhibits activity of miR-206. Analysis of the percentage of expression of miR-206 target for the control, tsg101, vps36, hrs, alix, and GW 182. Cells were transfected with 50 nM siRNA, plasmids expressing miR-206 or miR-K12-4 and the estrogen receptor 3′UTR inserted in the dual luciferase plasmid psiCHECK™ (Promega) (n=4). Thirty hours later luciferase activity as assessed.

FIG. 5: Differential centrifugation enriches exosomes greatly. (A) Western blot of 50 μg of cell or exosome lysate with markers of exosomes (CD63, Tfr R, or Ub). (B) Cell counts and total protein quantitation of exosome pellets 36 h after transfection of cells with siRNA targeting BIG2 or control. (C) Electron microscopy of resuspended exosome pellets after uranyl acetate staining. (D) Dynamic light scattering analysis of purified exosome pellets resuspended in PBS (Phosphate buffered saline). Top graph represents five sets of measurements as size in nm (x-axis) as a function of relative number (y-axis) of particles detected. Bottom graph represents size in nm (x-axis) as a function of volume (y-axis). Measurements by volume exponentially amplify the representation of larger particles present.

FIG. 6: Exosomes are enriched in GW182 and contain miRNA. (A) Western blot of equal amounts of protein from total cell lysate and exosomes with antibodies recognizing GW182, Ago2, Dcp1a, and Ge-1. (B) PNK labeled RNA from two preparations of exosomes compared to total cell RNA on a 15% acrylamide gel. (C) Sequences of cloned 19-33 nucleotide RNA from exosomes.

FIG. 7: exosomes transfer active siRNA and miRNA to target cells in a BIG2-dependent manner. (A) Exosomes purified from cells transfected with GFP siRNA or control siRNA were incubated with cells transfected with GFP. GFP expression was examined 8 hours or 24 hours later by flow cytometry. GFP expression was calculated as 1-(GFP expression GFP siRNA exosomes/GFP expression control siRNA exosomes)×100. (n=3, p<0.05, paired t-test). (B) Exosomes purified from cells transfected with miR-206 or miR-K12-4 were incubated with cells transfected with a target site for miR-K12-4 inserted into the psiCHECK dual luciferase plasmid. Activity of luciferases was measured 8 hours later. Inhibition of Firefly luciferase expression was calculated as: 1-(Firefly luciferase/Renilla luciferase miR-K12-4 exosomes)/(Firefly luciferase/Renilla luciferase miR-206 exosomes)×100. (n=4, p<0.05, paired t-test). Error bars represent standard error of the mean.

FIG. 8: Exosomes mediate transfer of miRNA activity in a BIG2-dependent manner. Exosomes purified from cells transfected with miR-206 or miR-K12-4 and siRNA targeting BIG2 or control were incubated with cells transfected with a psiCHECK dual luciferase plasmid expressing firefly luciferase with an inserted target site for miR-K12-4. In other controls, cells expressing psiCHECK reporter were incubated with naked plasmid for miR-206 or miR-K12-4. Activity of luciferases was measured after 8 hours (n=4, p=0.0287, t-test, a=0.05). Below, left. Exosomes from cells transfected with siRNA targeting GFP or control siRNA, or naked untransfected siRNA were incubated with cells transfected with GFP. GFP expression was measured by FACS analysis after 8 or 24 h (right).

FIG. 9: Purified exosomes are enriched in GW182, but not Dcp1a or Ge-1. (a) Equal amounts of proteins from exosomes and cells were analyzed by western blotting for exosome-enriched protein, CD63. Electron microscopy of resuspended exosome pellets. (b) Dynamic light scattering of exosomes demonstrates a single population of appropriate size (20-90 nm). Error bars, SEM of five measurements. (c) RNAi of BIG2 reduces the recovery of proteins in exosome preparations (paired t-test, n=4, p=0.0194, a=0.05). Analysis of the percent of cell number for BIG2 vs control and of the percent of exosome pellet for BIG2 vs control. (d) Equal amounts of proteins from exosomes and whole cells were analyzed by western blotting. (e) Exosomes were labeled with anti-GW182 mAB 4B6 and observed by electron microscopy (f) Western blot of crude membranous (16000 g 15 min) and cytoplasmic (supernatant) fractions. (g) reporter constructs showing let-7a interaction with target 3′UTR fused to Renilla luciferase (adapted from 5). mRNA repressed mRNA is lacking in cytoplasmic, but not membranous fractions (16000 g, 45 min, Actin n=3 p=0.0390, RRM2 n=3 p=0.0225).

FIG. 10: GW182 but not Dcp1a co-localizes with the multivesicular body. (a) Cells were loaded with NRhPE after transfection with plasmid expressing (a) YFP-CD82, a MVB-enriched protein (b, c) GFP-GW182, (d) GFP-Ago2, or (e,f) Dcp1a. (g,h) Cells were transfected with GFP-GW182 and RFP-Dcp1a. Scale bars=2 μM. RFP or NRhPE is shown in red (left column), GFP or YFP-tagged proteins in green (middle column), and co-localization appears in yellow in merged panels (right column).

FIG. 11: Exosomes contain mature miRNA, but lack miRNA-targeted mRNA. (a) Pie-chart representing the identity of cloned small RNA sequences in purified exosomes, as a percentage of sequences matching genomic human DNA. (b) Size distribution of all miRNA cloned from exosomes demonstrates a population with a median at 21-22 nucleotides (c) Exosomes contain miRNA profiles similar to whole cells. (d) mRNA repressed mRNA is excluded from exosomes compared to cells (Actin n=4 p=0.0019, RRM2 n=3 p=0.006). (e) Post-hoc bioinformatic analysis demonstrates that validated mRNA targets of miRNA are underrepresented in exosomes (13.63%, expected 14, χ2=6.636, p=0.01), and housekeeping genes undergoing less miRNA targeting are over-represented in exosomes (35.89%, χ2=16.641, p<0.0001). Error bars demonstrate confidence intervals (95%, modified Wald method).

FIG. 12: Knockdown of ESCRT complex components compromises miRNA activities. (a) GW182, but not Dcp1a associates with ubiquitinated proteins. Immunoprecipitating antibody is shown above lane, blotting antibody is inscribed in white (b) Knockdown of ESCRT complex components causes accumulation of GW182. (c) Co-localization of GFP-GW182 and N-Rh-PE was examined 36 h after siRNA transfection. (d) Knockdown of some components of the ESCRT complex inhibits activity of Let-7a (Anova, n=4, p=0.003, F=4.854, a=0.05), but does not change accumulation of Let-7a. U6 RNA was used as a loading control. (e) Knockdown of some members of the ESCRT complex inhibits activity of miR-206. (Anova, n=3, p=0.015, F=6.577, a=0.05). Lack of detectable miR206 expression in monocytic cells and accumulation of miR206 upon monocyte transfection were confirmed by northern blot.

FIG. 13: Enrichment of GW182 in exosome-like vesicles independent of bovine serum and in cells that are not of immune lineage. (a) GW182 but not Dcp1a is enriched in exosomes prepared from monocytic cells cultured in serum-free media (X-Vivo-15) or (b) Hela cells.

FIG. 14: Additional confocal microscopy images. Confocal microscopy of free GFP (top), the endoplasmic reticulum-specific protein sec61b-GFP (middle) and NRhPE, or (bottom) RFP-Gag from HIV-1 and GFP-Ago2. Scale bars=2 μM.

FIG. 15: Confocal micrographies: Localization of endogenous GW182, Dcp1a and MVB (CD63) by immunfluorescence. (a) A proportion of foci labeled with anti-human GW182 anti-serum 18033 in Mono-Mac-6 cells co-localizes with MVB (white arrowheads highlight co-localized foci). Note that since anti-GW182 anti-serum recognizes Ago and Ge-116, in addition to GW182, it should label both P-bodies and GW-bodies. (b) Less co-localization with MVB was observed with anti-Dcp1a antibody. (c) 293T cells co-labelled with anti-GW182 mAb 4B6 and anti-Dcp1a as previously described 24.

FIG. 16: Confocal micrographies: GW182 but not Dcp1a co-localizes with the multivesicular body in Hela cells. (a) Co-localization in Hela cells of GFP-Ago2, GFP-Dcp1a, or GFP-GW182 with N-Rh-PE, or GFP-GW182 with RFP-Dcp1a. Scale bars=2 μM A higher degree of co-localization of RFP-Dcp1a with GFP-GW182 and MVB was observed in Hela, as opposed to monocytic cells (58% vs. 6%, respectively). Nonetheless, GFP-GW182 punctuate structures co-localized with the MVB marker N-Rh-PE in Hela cells much more frequently than Dcp1a (68% vs. 15%). (b) A small proportion of cells showed little co-localization between GFP-Ago2 and MVB. Examples of Hela cells exhibiting poor co-localization of GFP-Ago2 with N-Rh-PE or significant co-localization of RFP-Dcp1a with MVB (middle) and GFP-GW182 (bottom). Scale bars=2 μM.

FIG. 17: Confocal micrographies: Localization of GFP-GW182 to the MVB is independent of stress granule formation. Co-localization of GFP-GW182 with N-Rh-PE was examined alone (a) or after co-transfection with (b) TIA-1 dominant negative or (c) a constitutively active eIF2a mutant (S51D), which respectively inhibit or drive the formation of stress granules33. Scale bars=2 μM.

FIG. 18: Table of mRNA of known miRNA targets or housekeeping genes showing enrichment in exosomes compared to cells. Microarray data derived from demonstrating the ratio of mRNA in exosomes versus cells of known-targets of miRNA and housekeeping genes. mRNA highlighted in red exhibit enrichment in exosomes compared to cells; those in green are diminished in exosomes.

FIG. 19: Verification of Efficacy of SiRNA Treatments. (a) cDNA was prepared from monocytic cells 30 hours after transfection with the designated siRNA. Thirty cycles of PCR were performed with primers to amplify b-actin and the appropriate mRNA. Forty-eight hours after treatment with 10 nM siRNA Hela cells were assayed for (b) mRNA levels as in (a). Knockdown in Hela cells of protein levels (alix, hrs siRNA), or inhibition of EGFR degradation (tsg101, vps36, hrs, PTPN23) was used to verify efficient inhibition of protein production and protein function respectively by siRNA. ESCRT complex proteins, excepting Alix, are required for efficient downregulation of EGFR after its ligation with EGF.

FIG. 20: Efficient miRNA activity requires some ESCRT complex proteins in Hela cells. Efficient miRNA activity in Hela cells requires select members of the ESCRT complex. Dual luciferase measurements were performed 48 h after transfection of cells with siRNA and reporter plasmids. ESR (One-way Anova, F=10.82, p=0.0004), Let-7a (One-way Anova, F=9.46, p=2 E-07)

FIG. 21: shows the efficiency of miRNA activity in Hela cells requires select members of the ESCRT complex. Dual luciferase measurements were performed 48 h after transfection of cells with siRNA and reporter plasmids. (a) Cells were co-transfected with Psicheck dual luciferase reporter with firefly luciferase linked to the 3′UTR of the estrogen receptor (ESR, contains a target site for miR-206) and a second plasmid expressing either a miR-206 or miRK12-4 precursor (One-way Anova, F=10.82, p=0.0004), Values are normalized to Renilla luciferase expression. (b) Cells were transfected with plasmid pGL3 expressing firefly luciferase and a second plasmid expressing Renilla luciferase attached to two artificial let-7a target sites, or mutated let-7a target sites (One-way Anova, F=9.46, p=2 E-07). Values are normalized to Firefly luciferase (c) Unnormalized or raw values for dual luciferase assays performed using the let-7a and miR206 reporters in Mono-Mac6 and Hela cells.

### EXAMPLES

### Example 1

### Delivery of Active siRNA and miRNA to Cells

### Materials and Methods

Antibodies

Antibodies were obtained as follows: Rabbit and mouse Immunoglobuline G (Sigma-Aldrich, St. Quentin Fallavier, France), anti-mono and poly-ubiquitinated proteins clone FK2 [Tebu-bio, Le Perray en Yvelines, France], anti-Dcp1a rabbit polyclonal (a kind gift of J. Lykke-Andersen, University of Colorado), anti-GW182 (serum 18033), anti-Ge-1 (serum 106), and normal human serum (kind gifts of M. Fritzler, University of Calgary).

Cell Culture

The Mono-Mac6 cell line (DSMZ, Braunschweig, Germany ACC-124) was cultured in RPMI 1640 (Roswell Park Memorial Institute 1640 buffer) containing 10% FBS (Phosphate buffered saline), non-essential amino acids (Invitrogen, Paris, France), and OPI (oxaloacetate, pyruvate, and bovine insulin) media supplement (Sigma-Aldrich).

Enrichment of Exosomes

Cells were grown at a density of 0.5-1.0×10⁶ cells/mL for 8 to 24 h. Cells were centrifuged at 400 g for 5 min and supernatant was removed. The same process was repeated with centrifugations at 1200 g (5 min), 10 000 g (30 min). Following centrifugation at 100 000 g (1 h, SW27 rotor, Beckman-Coulter, Roissy, France) all supernatant was removed with a micropipette and the pellet was recovered in PBS for subsequent analyses.

Confocal Microscopy

Cells were concentrated by centrifugation (90 g, 5 min), resuspended in culture media, and analyzed immediately. Images were captured with a Zeiss LSM 510 confocal microscope with 488 nm and 561 nm lasers using the smallest planes (0.4-1.2 μm) allowed by the brightness of the fluorophores used and a 63× objective lens. Filters used were 500-530 nm (GFP) and 550-650 (N-Rhodamine-PhosphatidylEthanolamine, rhodamine fluorescent protein (RFP)). Images were analyzed using ImageJ.

Cell Loading with N-Rhodamine-PhosphatidylEthanolamine (N-Rh-PE)

Procedures were performed as described (Vidal, M., Mangeat, P., & Hoekstra, D. Aggregation reroutes molecules from a recycling to a vesicle-mediated secretion pathway during reticulocyte maturation. J. Cell Sci. 110, 1867-1877 (1997) [46]). Essentially, cells were washed twice in PBS and 3 μM N-Rh-PE (Avanti Polar Lipids, Alabaster, Ala., USA) in ethanol was injected with a Hamilton syringe into the cell suspension. After vortexing cells were incubated for 1.5 h at 4° C. Cells were washed twice, resuspended in culture media and incubated at 37° C. with 5% CO2 for 4 h before confocal analysis.

Plasmids and siRNA

Plasmids expressing GFP-hAgo2 (Jakymiw, A. et al. Disruption of GW bodies impairs mammalian RNA interference. Nat. Cell Biol. 7, 1267-1274 (2005) [47]) (11590), YFP-CD82 (Sherer, N. M. et al. Visualization of retroviral replication in living cells reveals budding into multivesicular bodies. Traffic. 4, 785-801 (2003) [48]) (1819), (1817), Gag-RFP [48] (1814), Sec616-GFP (Voeltz, G. K., Prinz, W. A., Shibata, Y., Rist, J. M., & Rapoport, T. A. A class of membrane proteins shaping the tubular endoplasmic reticulum. Cell. 124, 573-586 (2006)[49]) (15108) were obtained via Addgene (Cambridge, Mass., USA). Plasmids expressing GFP-GW182-GFP [3] (Ed Chan, University of Florida, USA), a dominant negative version of TIA-1 (Kedersha, N. L., Gupta, M., Li, W., Miller, I., & Anderson, P. RNA-binding proteins TIA-1 (T-cell intracellular antigen 1) and TIAR (T-CELL restricted intracellular antigen related protein) link the phosphorylation of eIF-2 alpha to the assembly of mammalian stress granules. J. Cell Biol. 147, 1431-1442 (1999) [7]) (Nancy Kedersha Brigham and Women's Hospital and Harvard Medical School, Boston, Mass., USA), and a constitutively active version of eIF2a [7] (Randall Kaufmann, University of Michigan, Ann Arbor, Mich., USA) were kind gifts of Nancy Kedersha.

SiRNA confirmed to knockdown mRNA expression by at least 70% were obtained from Qiagen (Courtaboeuf, France). These were (all the references are catalog numbers from Qiagen that designate the specific siRNA): Hrs (SI00288239 CCGGAACGAGCCCAAGTACAA*, SiRNA were obtained from Qiagen (Courtaboeuf, France) unless otherwise indicated. * denotes an siRNA validated by the company to knockdown mRNA expression of the respective gene by at least 75%. These were: Hrs (CCGGAACGAGCCCAAGTACAA*), GW182 (TN RC6A, 8103648743: AAGAGCTTAACTCATCTTTAA*), Alix (SI02655345: AAGAGCTGTGTGTTGTTCAAT*), Vps36 (CCCGATCAATTGAGAATTTAT*), Tsg101 (SI00318045), GFP (1022064, siRNA GAACUUCAGGGUCAGCUUGCCG, SEQ ID NO. 23). All Stars Negative Control siRNA (this sequence is not revealed, it is an industry secret 1027281).

Immunoprecipitations

Cells were lysed at a concentration of 20×10⁶ cells/mL in 0.5% NP-40 (nonyl phenoxylpolyethoxyl ethanol 40), 10% glycerol, in PBS with 20 μM MG132 (Proteasome inhibitor) and Complete protease inhibitor cocktail (Roche, Meylan, France). After 20 minutes rotating at 4° C. lysate was centrifuged at 16 000 g for 30 minutes. Antibody was added to lysates (1/400 for GW182 and Ge-1, and control normal human serum; 2 μg mouse IgM or anti-ubiquitin FK2; 1/400 anti-Dcp1a and an equivalent amount of rabbit Ig [determined by ELISA measurement of Dcp1a concentration]). After 3 h protein G agarose (Roche, Meylan, France) or anti-mouse IgM-agarose (Sigma-Aldrich) were added for 1 h. Three washes were performed with lysis buffer, centrifuging at 12 000 g for 1 min.

Electron Microscopy

Purified exosomes were left to settle on nickel coverslips (100 mesh, EMS, Pennsylvania, USA) that were coated with a 0.25% Formvar film (EMS). After staining with 2% uranyl acetate for 30 seconds coverslips were left to dry and visualized using a transmission electron microscrope (Hitachi H600, 75 KV).

Dynamic Light Scattering

Purified exosomes resuspended in DPBS (Dulbecco's Phosphate Buffered Saline) (Invitrogen) were analyzed with a Zetasizer Nano S from Malvern Instruments (Malvern, UK). Samples were loaded in 40 microL quartz cuvettes and five measurements were performed in automatic mode after an equilibration time of 2 min at 20° C. Experimental data were processed in multiple narrow modes assuming that particles are spheres and corrected for solvent refractive index and viscosity (respectively 1.332 and 1,029 as calculated from solvent composition). A mean exosome diameter was derived from the plot of the size distribution as a function of the intensity of scattered light.

PNK, Low Molecular Weight RNA Gel

Total RNA was extracted from exosomes (derived from 100×10⁶ cells) in 100 μL PBS with Trizol LS (Invitrogen) and resuspended in 10 μL water. Two μL exosome RNA or 0.5 μL total RNA was incubated with 1 μL T4 Polynucleotide Kinase in exchange buffer B (Fermentas, St. Remy les Chevreuse, France) and SpCurie γATP at 37° C. for 1 h in a total volume of 20 μL. Forty μL water was added and excess γATP (adenosine triphosphate was removed on a G25 mini-column (GE Healthcare, Orsay, France), RNA was run on a 15% polyacrylamide gel containing 6 M urea as previously described.

Library Cloning

Small RNA cloning was performed as described (Pfeffer, S. Identification of Virally Encoded MicroRNAs. Methods Enzymol. 427:51-63., 51-63 (2007) [50]) using 200 μg of total RNA for the library from MonoMac-6 cells and 2 μg total RNA from exosomes. Libraries were sequenced using 454 technology (www.454.com). Sequences were annotated as described (Pfeffer, S., Lagos-Quintana, M., & Tuschl, T. Cloning of small RNA molecules. Curr. Protoc. Mol. Biol. Chapter 26:Unit 26.4., Unit (2005) [51]) using the following databases: genomic sequences were from the UCSC Genome Browser database (NCBI build 37, July 2007). tRNA (transfer ribonucleic acid), rRNA (ribosomal ribonucleic acid), snRNA (Small nuclear ribonucleic acid), snoRNA (Small nucleolar ribonucleic acid), and scRNA (small cytoplasmic ribonucleic acid) sequences were extracted from the release 158 of Genbank (Feb. 15, 2007).

Tests of MiRNA Activity

Cells were transfected with 0.2 ug plasmid containing the 3′UTR (3′ untranslated region) of ESR (Estrogen Receptor) containing a target for miR-206 (Adams, B. D., Furneaux, H., & White, B. A. The micro-ribonucleic acid (miRNA) miR-206 targets the human estrogen receptor-alpha (ERalpha) and represses ERalpha messenger RNA and protein expression in breast cancer cell lines. Mol. Endocrinol. 21, 1132-1147 (2007) [13]), 2 μg of either plasmid expressing miR-206 or K12-4, and either control siRNA or siRNA targeting tsg101, vps36, hrs, alix, or GW182 (50 nM). Luciferase activity was read 30 h later, using the Dual Luciferase Reporter Assay Kit (Promega, Madison, USA) on a Glo-Max Multi fluorescence reader (Promega). Percent increase in siRNA activity was calculated as (e.g. tsg101): ([Firefly/Renilla luciferase] K4 miRNA+tsg101 siRNA/[Firefly/Renilla luciferase]+206 miRNA+tsg101 siRNA)/([Firefly/Renilla luciferase]+miRNA K4+control siRNA/[Firefly/Renilla luciferase]+miRNA 206+control siRNA).

Intercellular Transfer of siRNA and miRNA

One group of cells was transfected with 250 nM GFP siRNA or control siRNA and cultured for 8 h before isolation of exosomes. Exosome pellets were resuspended in 1.2 mL X-VIVO 15 media (Lonza, St. Beauzire, France). A second group of cells was transfected with 1 μg plasmid expressing eGFPN1. One hour after transfection of EGFPN1 plasmid, cells were washed twice in X-VIVO 15 media (Lonza, St. Beauzire, France) and 0.4×10⁶ cells were resuspended in 500 uL exosome-containing X-VIVO 15. % inhibition of target expression in target cell was calculated gating on GFP+ cells as 100−(geometric mean exosomes containing GFP siRNA/geometric mean exosomes containing control siRNA). Experiments of miRNA transfer by exosomes were repeated identically as above excepting the transfection of plasmid expressing miR-206 or mir-K12-4 into exosome producing cells, and the transfection of psiCHECK plasmid containing a target site for miR-K12-4 into target cells. Control siRNA or BIG2 siRNA were co-transfected with miR-206 and miR-K12-4. Percent inhibition of firefly luciferase expression was calculated as Firefly/Renilla luciferase with exosomes containing miR-K12-4)/(Firefly/Renilla luciferase exosomes containing miR-206)

Transfections

For all experiments except for assays of miRNA activity 5×10⁶ cells were transfected with a Nucleofector II in Solution V (Amaxa, Cologne, Germany) according to the manufacturer's instructions. For inhibition of gene expression 50 nM of siRNA was used. For experiments evaluating miRNA activity 1×10⁶ cells were transfected in 100 μL culture media lacking FBS using 250 μg/mL DEAE-dextran (Promega, Charbonnieres, France). Cells were incubated 1.5 h at 37° C. Ten μL DMSO was added. After three minutes 2 mL of cell culture media was added, cells were centrifuged and resuspended in RPMI 1640 containing FBS for 30 h.

Results

Components of the miRNA Machinery Co-Localize with the Multivesicular Body

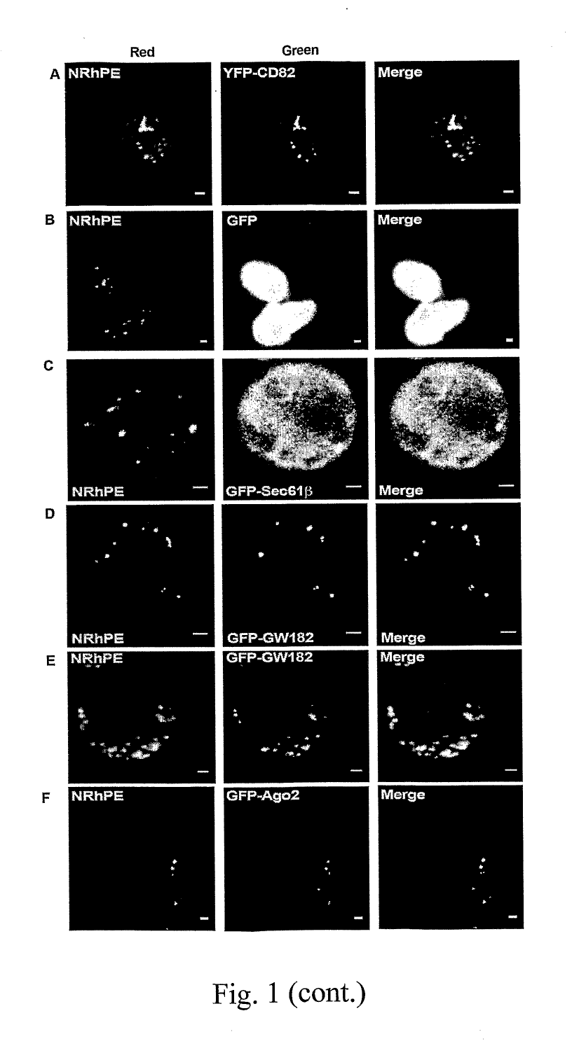

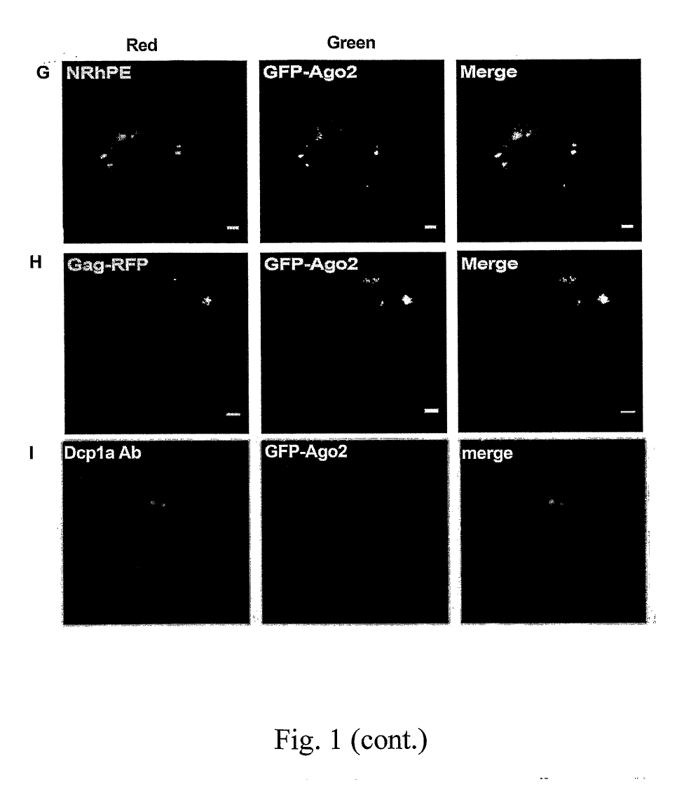

Autoantibodies to phosphatidylethanolamine co-localize with autoantibodies to GW182 [5], and knockdown of Gawky the drosophila GW182 homologue results in enlarged MVB [2]. N-rhodamine labeled phosphatidylethanolamine (N-Rh-PE) selectively accumulates in the MVB (Vidal, M., Mangeat, P., & Hoekstra, D. Aggregation reroutes molecules from a recycling to a vesicle-mediated secretion pathway during reticulocytematuration. J. Cell Sci. 110, 1867-1877 (1997) [6]). We therefore tested whether Ago2 and GW182 co-localize with N-Rh-PE and the MVB. Accurate labeling of MVB by N-Rh-PE was confirmed by co-localization with CD82 (FIG. 1A) and the absence of co-localization with Sec61β or GFP (FIG. 1B,C). GFP-GW182 labeled punctuate structures a significant proportion of which co-localized with N-Rh-PE. Similar results were found with GFP-Ago2 (FIG. 1F,G). Ago2-GFP also co-localized with Gag, another protein selectively accumulated in MVB (FIG. 1H).

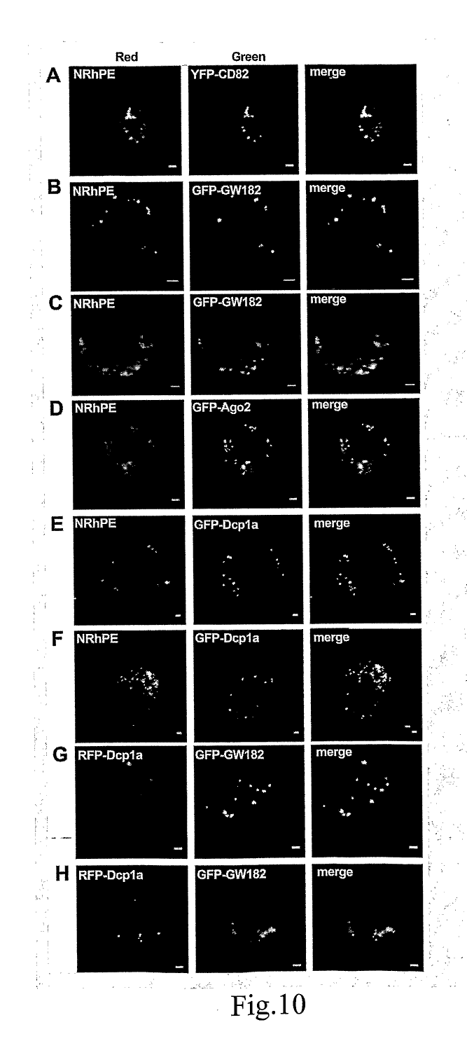

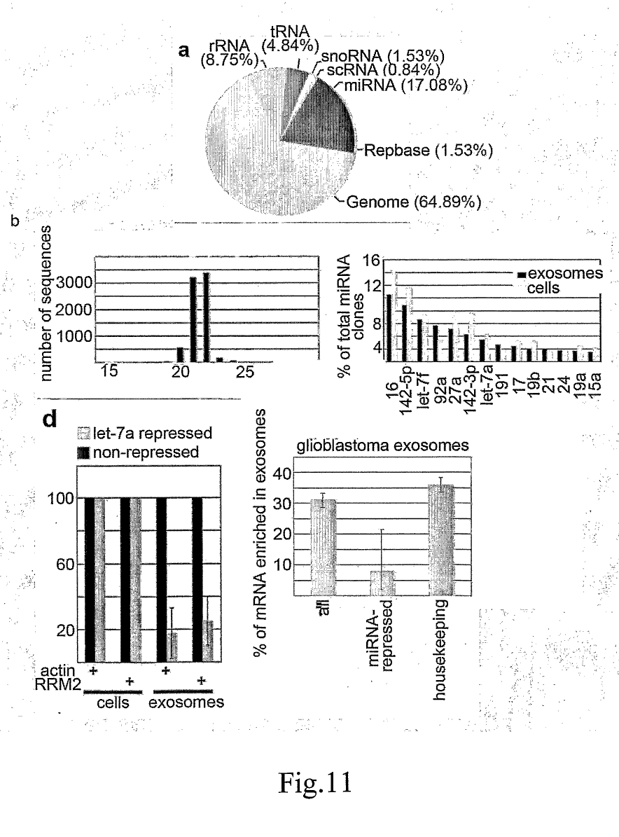

To confirm that the localization of Ago2-GFP observed in transiently transfected cells corresponds to that expected for endogenous Ago2, cells were lightly fixed, permeabilized and stained with anti-Dcp1a Ab to mark P-bodies. Ago2-GFP co-localized with endogenous Dcp1a as expected (FIG. 11).

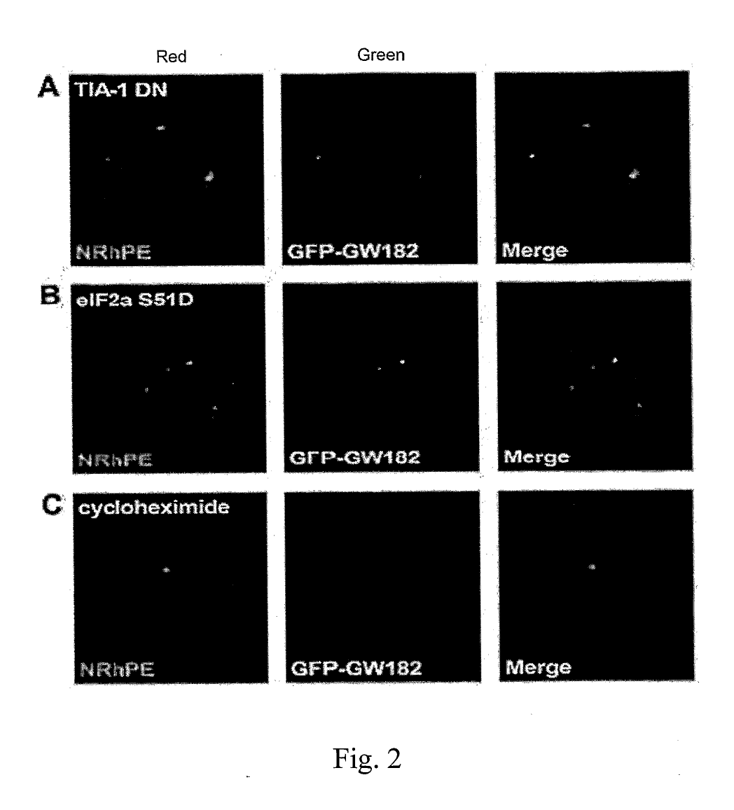

Components of the miRNA machinery, such as Ago2, can co-localize with stress granules. We investigated whether GFP-GW182 and GFP-Ago2 co-localized with the MVB as part of stress granules. Neither induction of stress granules with a constitutively active eiF2a mutant, or inhibition of stress granules with a dominant negative version of TIA-1 [7] modified the co-localization of GFP-GW182 with the MVB (FIG. 2A, B). This suggests that GW182 and Ago2 are not co-localized with the MVB as part of the classic stress response that generates stress granules. Indeed, GW182 is believed to be localized only in P-bodies and not in stress granules (Kedersha, N. & Anderson, P. Mammalian stress granules and processing bodies. Methods Enzymol. 431:61-81., 61-81 (2007) [8]).

P-bodies are disassembled upon treatment with cycloheximide [8]. In accordance, fewer or no punctuate GW182+ structures were observed co-localized with the MVB in cycloheximide treated cells (FIG. 2C). Thus, a significant pool of GW182 and Ago2 co-localizes with the MVB as part of P-body-like structures.

Thus it appears that many essential components to the small RNA machinery are localized in a specific organelle, namely the MVB.

GW182 is Ubiquitinated.

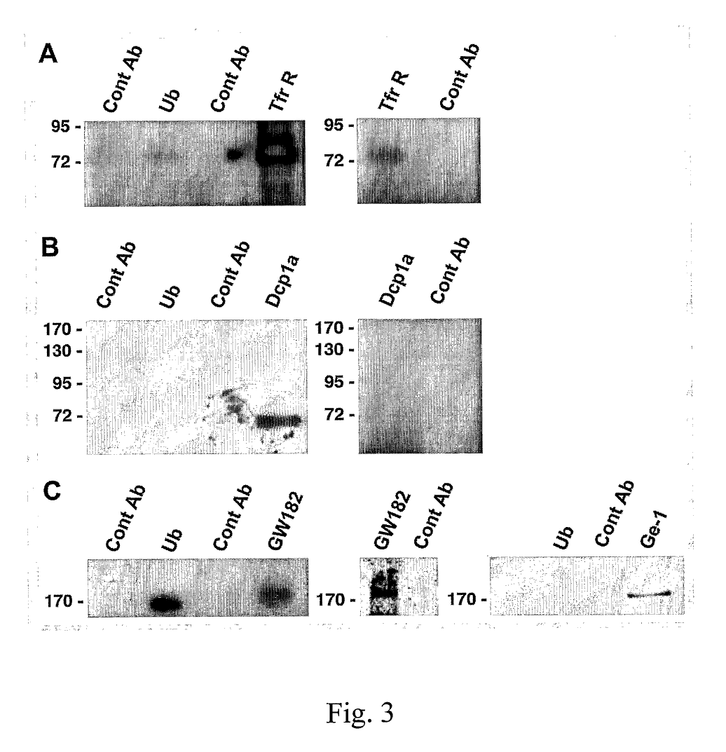

A major mechanism of sorting proteins to the MVB is the ESCRT complex which binds ubiquitinated proteins. We tested several components of the RNA silencing machinery for association with ubiquitinated proteins or for direct ubiquitination by immunoprecipitation with specific or anti-Ub antibody. Transferrin receptor (TfrR), as a positive control, could be detected among proteins immunoprecipated with an anti-Ub antibody, and conversely, immunoprecipitated TfrR could be detected with an anti-Ub antibody (FIG. 3A). Ubiquitinated proteins were faintly detected in Dcp1a immunoprecipitates. These proteins were inconsistent with the molecular mass of Dcp1a and Dcp1a was not detected among proteins immunoprecipitated with anti-Ub antibody (FIG. 3B). Therefore Dcp1a may weakly or transiently interact with some ubiquitinated proteins but is probably not directly ubiquitinated. GW182 has a UBA domain that in other proteins often binds ubiquitin, and proteins with UBA domains are often ubiquitinated themselves (Peschard, P. et al. Structural basis for ubiquitin-mediated dimerization and activation of the ubiquitin protein ligase Cbl-b. Mol. Cell. 27, 474-485 (2007) [9]). Anti-Ub antibody immunoprecipitated a protein reactive with anti-GW182 antiserum (FIG. 3C) and anti-GW182 antibody immunoprecipitated a ubiquitinated protein consistent with the size of GW182 (FIG. 3C). While predominantly recognizing GW182, anti-GW182 serum also contains antibodies recognizing Ge-1. Ge-1 was not detected among ubiquitinated proteins (FIG. 3D) suggesting that GW182 and not Ge-1 is the ubiquitinated and ubiquitin-associated protein detected.

The ESCRT Pathway has a Negative Effect on siRNA and miRNA Activity

Localization of components to the MVB may provide a means to efficiently regroup mRNA, miRNA and/or protein components of the silencing machinery. We hypothesized that disrupting this localization may affect miRNA activity.

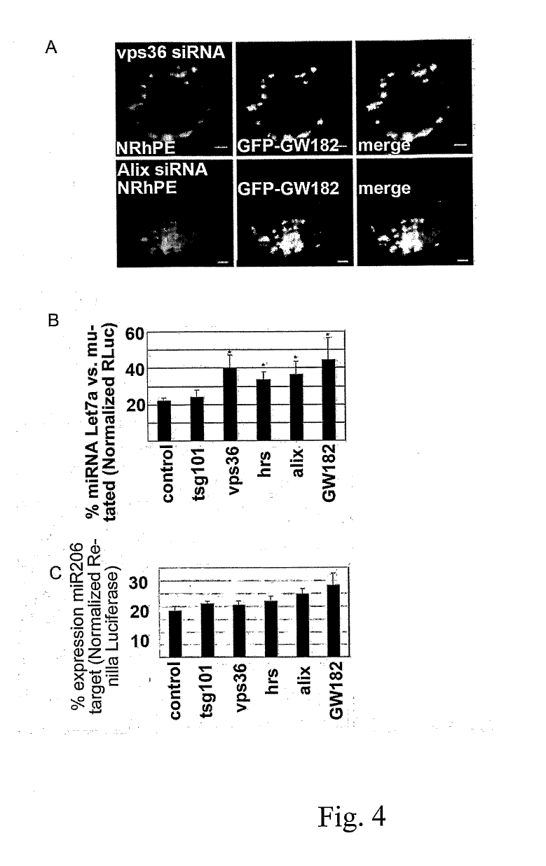

As previously described, siRNA targeting Alix or vps36, components of the ESCRT complex, induced an increased size of MVB (siRNA targeting vps36) or an increase in the perinuclear distribution of MVB (siRNA targeting Alix) in some cells (Cabezas, A., Bache, K. G., Brech, A., & Stenmark, H. Alix regulates cortical actin and the spatial distribution of endosomes. J. Cell Sci. 118, 2625-2635 (2005) [11]). Nonetheless co-localization of GW182 with these altered MVB was not grossly disrupted (FIG. 4A). Despite this, we tested the activity of miRNA in cells in which components of the ESCRT complex had been targeted by siRNA. Knockdown of hrs, another component of the ESCRT complex, vps36 or alix, but not tsg101 (supplementary FIG. 1) significantly inhibited the activity of let-7 compared to a control with 2 nucleotide changes in the let-7 site (FIG. 4B) (Doench, J. G. & Sharp, P. A. Specificity of microRNA target selection in translational repression. Genes Dev. 18, 504-511 (2004) [12]). Inhibition of miRNA activity by components of the ESCRT complex, in particular alix, approached that attained upon knockdown of GW182 (FIG. 4B).

To ensure that the effect of the ESCRT complex on miRNA activity was not limited to let-7 or the previous reporter system, we performed similar experiments with a second system. A plasmid expressing miR-206 or Kaposi's Sarcoma Virus (KSHV) miRNA K-12-4 were transfected into cells along with a reporter linked to an endogenous target of miR-206, the estrogen receptor-α3′UTR [13]. In agreement with the previous system, knockdown of alix or hrs, but not tsg101 inhibited the specific activity of miR-206.

Characterization of Exosomes

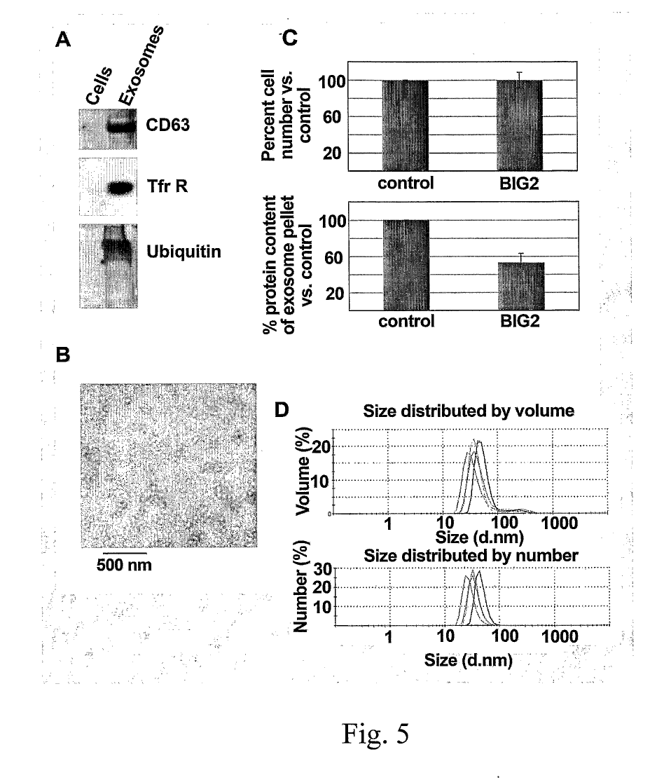

Since components of the miRNA pathway localize to the MVB it was possible that they were packaged into ILV in the MVB and released into the extracellular space as what are often termed exosomes. To examine this possibility we first undertook to characterize the identity and purity of exosomes purified by established protocols in our hands. Purified exosomes were highly enriched in transferrin receptor, CD63 and ubiquitinated proteins, all classic markers of exosomes (Buschow, S. I., Liefhebber, J. M., Wubbolts, R., & Stoorvogel, W. Exosomes contain ubiquitinated proteins. Blood Cells Mol. Dis. 35, 398-403 (2005) [14], Thery, C., Zitvogel, L., & Amigorena, S. Exosomes: composition, biogenesis and function. Nat. Rev. Immunol. 2, 569-579 (2002) [15]) (FIG. 5A). Purified exosomes contained vesicles of the expected size and morphology when visualized by electron microscopy (FIG. 5B). To establish the purity of exosomes in our preparations we used two methods. First, siRNA targeting Brefeldin A-Inhibited Guanine nucleotide-exchange protein (BIG2, knockdown shown in supplementary FIG. 1), which is important for release of exosome-like vesicles (Islam, A. et al. The brefeldin A-inhibited guanine nucleotide-exchange protein, BIG2, regulates the constitutive release of TNFR1 exosome-like vesicles. J. Biol. Chem. 282, 9591-9599 (2007) [16]) reduced exosome release by almost 50% (FIG. 4C). Taking into account the modest level of transfection efficiency (˜50% with plasmid DNA) in the monocytic cells used, this suggests a large majority of the purified material consists of exosomes. Second, we used dynamic light scattering, to quantitate the homogeneity of purified exosomes. Purified material had an average size consistent with exosomes, were homogenous in size (a single population of low variance is visible), and had a relative purity of >99% (FIG. 4D). This suggests the exosomes purified by standard procedures are highly pure.

Components of the miRNA Pathway are Found in Secreted Exosomes

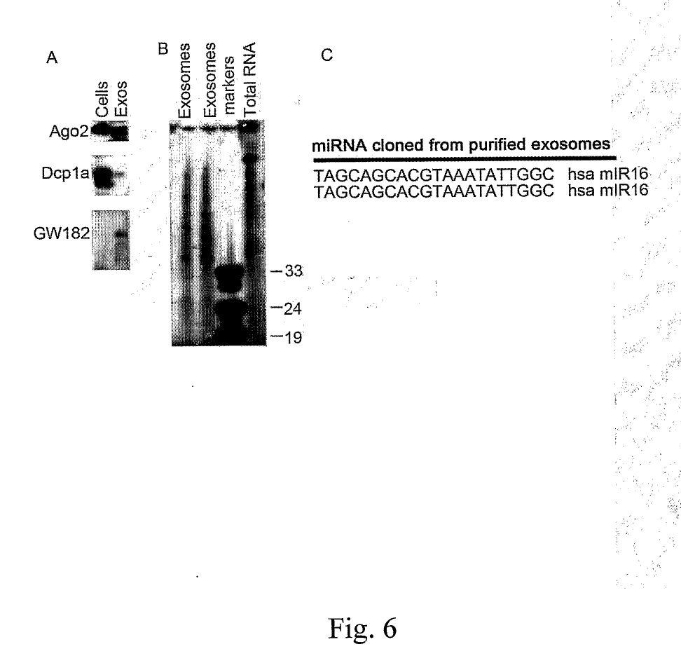

Since components of the miRNA pathway localize to the MVB they may eventually be secreted in exosomes. Using western blot we investigated the presence of protein components of RNA silencing in exosomes compared to equal amounts of proteins from a total cell lysate, as performed above for exosome positive controls. Ago2 was found in exosomes in amounts comparable to whole cell lysate. Strikingly, compared to total cell lysate, GW182 was dramatically enriched in exosomes (FIG. 6A), and Dcp1a was largely missing.

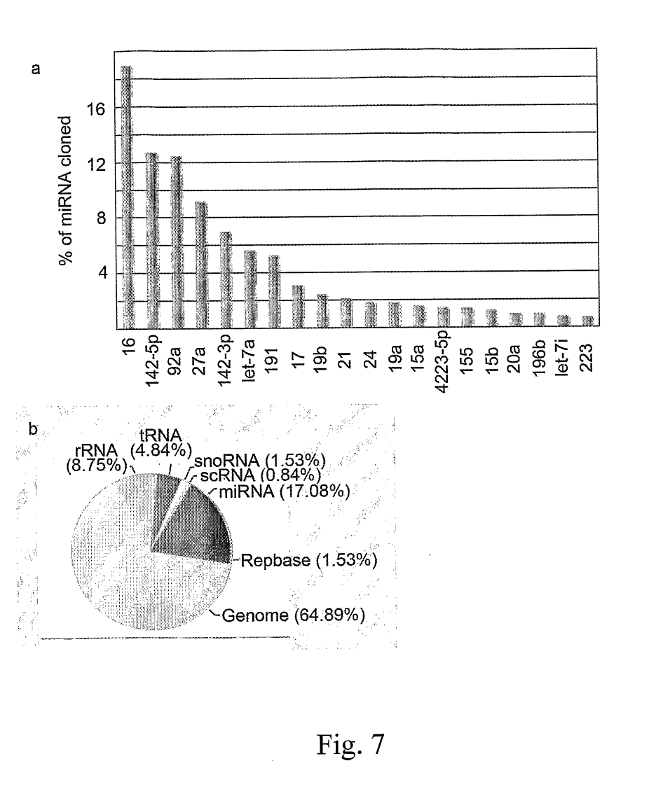

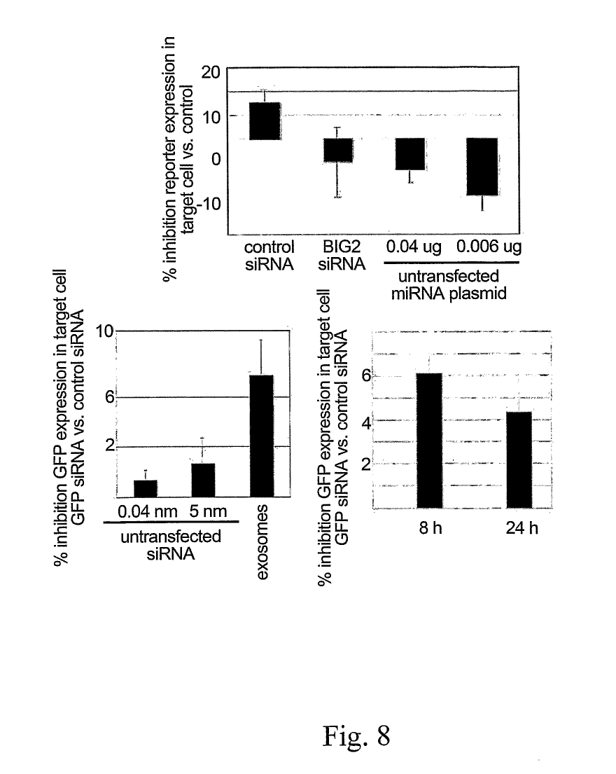

It had previously been shown that exosomes contained mRNA and microRNA 17. Separation of polynucleotide kinase labeled RNA on a 15% acrylamide gel allowed visualization of small RNA contained in exosomes and total cells. RNA from exosomes exhibited distinct enrichment of several bands of RNA compared to total cell RNA, suggesting a selective loading of some small RNA species in exosomes. A discrete population of RNA between 19 and 24 nucleotides in length consistent with miRNA was also observed within exosome RNA (FIG. 5B). A library of RNA 19-33 nucleotides in length was made from purified exosomes and total RNA from monocytes. Preliminary analysis of high throughput pyrosequencing showed the presence of significant amounts of miRNA (FIG. 7). For example, miR-16 and miR-27b were present in significant quantities [1.43% (106/7436) and 0.30% (22/7436)]. No sequences deriving from miR-16* were retrieved, suggesting loading of miRNA into exosomes occurs after strand disjoining. These results confirm that highly purified exosomes contain miRNA.

Intercellular Transfer of siRNA and miRNA

Exosomes are targeted to macrophages and dendritic cells and are subsequently endocytosed (Morelli, A. E. et al. Endocytosis, intracellular sorting, and processing of exosomes by dendritic cells. Blood. 104, 3257-3266 (2004) [18]). Proteins associated with exosomes can be degraded into peptides and presented on MHC class I of a cell that takes up exosomes [15]. This suggests exosomes, or some components of exosomes may escape to the cytosol before complete degradation in the lysosome. Because in plants and C. elegans intercellular transport of miRNA occurs (Voinnet, O, Non-cell autonomous RNA silencing. FEBS Lett. 579, 5858-5871 (2005) [19]) we tested whether siRNA or miRNA transported by exosomes could inhibit gene expression in a target cell. Cells were transfected with siRNA targeting GFP or control siRNA and exosomes were purified eight to 12 hours later and added to GFP-expressing cells. GFP expression was reduced by 7.3% in cells incubated with exosomes from GFP siRNA transfected cells compared to cells incubated with exosomes derived from cells transfected with control siRNA (FIG. 7A). The diminution of GFP expression by GFP siRNA exosomes increased with the number of resuspended exosomes added (FIG. 7A, 1.58% with 100 μL exosomes versus 7.3% with 500 μL exosomes). The effect of GFP siRNA in target cells had decreased slightly at 24 hours compared to 6 hours (FIG. 7B, 6.1% inhibition of GFP expression at 6 h versus 4.3% at 24 h).

We used a viral miRNA and target to test whether the intercellular transfer of small RNA observed would still occur if transcription and processing of the miRNA had to occur before packaging into exosomes. One batch of cells was transfected with plasmid expressing KHSV miR-K12-4 or control plasmid expressing miR-206. Incubation of exosomes containing miR-K12-4 with cells decreased expression of a miR-K12-4 target reporter by 15.3% compared to exosomes containing miR-206 (FIG. 7C). If knockdown of reporter expression in target cells is due to exosome-mediated transfer of miR-K12-4 then siRNA targeting BIG2 should block this effect by inhibiting release of exosomes (FIG. 4C) [16]. Delivery of an siRNA targeting BIG2 simultaneously with plasmids expressing miR-K12-4 or miR-206 inhibited knockdown of miR-K12-4 reporter expression in target cells by exosomes presumably containing miR-K12-4 or one of its precursors. Thus, exosome-like vesicles can transfer functional miRNA to a target cell in a BIG2-dependent manner.

Delivery of Active siRNA and miRNA to Cells

We demonstrate that a significant proportion of RNA silencing machinery co-localizes with the MVB. We suggest that at least two separable pools of RNA silencing machinery associate with the MVB. One pool, containing GW182, some Ago2 and miRNA but little Dcp1a is sequestered from cytoplasmic RNA in ILV. These ILV may be released as exosomes. The initial description of miRNA in exosomes ([17]) could have been interpreted as random loading of cytoplasmic contents. Given the co-localization of GW182 and Ago2 at the MVB, and the distinct enrichment of GW182 and lack of Dcp1a in exosomes, miRNA and potentially its mRNA targets may be subject to a selective sorting process for loading into exosomes. GW182 is highly enriched in exosomes, while little Dcp1a was present. This suggests a specific sorting mechanism exists that recruits some, but not all, components of P-bodies into exosomes. The UBA domain of GW182, and covalent ubiquitination of GW182 may drive its interaction with the MVB and sorting into exosomes. Conversely, the lack of ubiquitination of Dcp1a may disfavor its loading into exosomes. Ago2 associates with ubiquitinated proteins (Matsumoto, M. et al. Large-scale analysis of the human ubiquitin-related proteome. Proteomics. 5, 4145-4151 (2005) [20]), and binds GW182 (E1-Shami, M. et al. Reiterated WG/GW motifs form functionally and evolutionarily conserved ARGONAUTE-binding platforms in RNAi-related components. Genes Dev. 21, 2539-2544 (2007) [21]), suggesting a mechanism for its appearance in exosomes. Dcp1a may be dislocated from P-body complexes before GW182 is delivered into ILV, or potentially GW182 is packaged into exosomes and Dcp1a is packaged into distinct ILV for lysosomal degradation.

A second pool of RNA silencing machinery may be on the surface of the MVB in agreement with previous studies that biochemically described the membrane localization of Ago2 [4]. We hypothesize that localization of GW182 and Ago2 is an addressing mechanism at a certain stage of miRNA activity, whether that be for miRNA maturation, miRNA binding to Ago, target mRNA binding, a particular means of translational inhibition, decapping, or mRNA degradation and renewal of the RNA silencing machinery. Disruption of any of these steps could inhibit miRNA activity as observed upon knockdown of some components of the ESCRT complex.

ILV containing miRNA may be targeted for lysosomal degradation or extracellular release as exosomes. Messenger RNA and miRNA had previously been demonstrated in exosomes (Valadi, H. et al. Exosome-mediated transfer of mRNAs and microRNAs is a novel mechanism of genetic exchange between cells. Nat. Cell Biol. 9, 654-659 (2007) [17]), and in cell-free plasma (Chim, S. S. et al. Detection and characterization of placental microRNAs in maternal plasma. Clin. Chem. 54, 482-490 (2008) [22]). While the former study claimed to demonstrate transcription of exosomal mRNA in target cells several uncertainties persisted [17].

We confirm the presence of miRNA in exosomes and demonstrate for the first time that small RNA can be transferred in a functional form by exosomes to target cells, and that mature miRNA are the active entity transferred by exosomes.

According to the invention, the multivesicular body and ubiquitination may represent a platform for localization and activity of miRNA components across species. Not the UBA domain of GW182, but the glutamine-rich domain beside it is responsible for GW182 localization to P-bodies ([3], Behm-Ansmant, I. et al. mRNA degradation by miRNAs and GW182 requires both CCR4:NOT deadenylase and DCP1:DCP2 decapping complexes. Genes Dev. 20, 1885-1898 (2006) [23]), unless the UBA domain on the extremity of a deletion mutant is non-functional [23]. Since GW182 is ubiquitinated and contains a UBA domain, this may drive its self-association, as for Cbl [9] possibly driving scaffolding on ESCRT complexes. In C. elegans and plants components of the cytoskeletal machinery and two ARF proteins were identified as important for miRNA activity (Peter Brodersen and Olivier Voinnet, submitted) (Parry, D. H., Xu, J., & Ruvkun, G. A whole-genome RNAi Screen for C. elegans miRNA pathway genes. Curr. Biol. 17, 2013-2022 (2007) [24]), suggesting the possibility of vesicular traffic in miRNA activity. Interestingly, vesicles have been observed associated with oocyte sponge bodies (Wilsch-Brauninger, M., Schwarz, H., & Nusslein-Volhard, C. A sponge-like structure involved in the association and transport of maternal products during Drosophila oogenesis. J. Cell Biol. 139, 817-829 (1997) [25]) that are involved in transport of bicoid RNA. Vps36 is required for transport of bicoid RNA Orion, U. & St, J. D. bicoid RNA localization requires specific binding of an endosomal sorting complex. Nature. 445, 554-558 (2007) [26]) and we demonstrate here that this ESCRT complex protein affects miRNA activity.

Other components of the ubiquitin pathway and the MVB may be used in RNA silencing, for example MEX-3B (Dvorak, A. M. & Morgan, E. S. The case for extending storage and secretion functions of human mast cell granules to include synthesis. Prog. Histochem. Cytochem. 37, 231-318 (2002) [27]), TRIM (tripartite motif) or NHL (ring finger b-box coiled coil) family proteins (Schwamborn et al. Cell 2009 136: 913 [93]), and possibly Ro52 (Bhanji, R. A., Eystathioy, T., Chan, E. K., Bloch, D. B., & Fritzler, M. J. Clinical and serological features of patients with autoantibodies to GW/P bodies. Clin. Immunol. 125, 247-256 (2007) [28]), Zinc finger-RING type ubiquitin ligases, or other types of ubiquitin ligases recognizing particular constructions of proteins on a mRNA. Such ligases may be for example, AUF1 binding to a 3′UTR may target a miRNA-mRNA pair for ubiquitin-mediated degradation (Laroia, G., Sarkar, B., & Schneider, R. J. Ubiquitin-dependent mechanism regulates rapid turnover of AU-rich cytokine mRNAs. Proc. Natl. Acad. Sci. U.S.A. 99, 1842-1846 (2002) [29]). As well, nhl-2, and other proteins of the RBCC-Ring family, potential ubiquitin ligases may be used to bind Argonautes, and recruit mRNA storage machinery to let-7 targeted mRNA (Schwamborn et al. [93]). In agreement with this model, ubiquitin was found in Ago complexes II and III, retrieved from sucrose fractions with polyribosomes, PABP, and mRNA (Hock, J. et al. Proteomic and functional analysis of Argonaute-containing mRNA-protein complexes in human cells. EMBO Rep. 8, 1052-1060 (2007) [30]). Indeed, several components of the mRNA cap binding complex are regulated by ubiquitination (Dorrello, N. V. et al. S6K1- and betaTRCP-mediated degradation of PDCD4 promotes protein translation and cell growth. Science. 314, 467-471 (2006) [31]), suggesting that localized ubiquitination and sorting at the MVB could finely regulate inhibition of translational initiation by miRNA.

According to the invention, the vesicular traffic co-ordinated by the multivesicular body may be utilized for non-cell autonomous RNAi in plants and C. elegans. Exosome-like vesicles in the extracellular space were identified in plants recently associated with the MVB (An, Q., Huckelhoven, R., Kogel, K. H., & van Bel, A. J. Multivesicular bodies participate in a cell wall-associated defense response in barley leaves attacked by the pathogenic powdery mildew fungus. Cell Microbiol. 8, 1009-1019 (2006) [32]). Furthermore, genetic screens have found several proteins important for MVB formation, trafficking, or with functions in the ubiquitin pathway were required for RNAi. Rab7 is essential for RNAi in Drosophila and C. elegans. Rab7 is involved in trafficking to the MVB, and an effector of Rab7, RILP, interacts with vps36 and is also important for multivesicular body morphology and function (Wang, T. & Hong, W. RILP interacts with VPS22 and VPS36 of ESCRT-II and regulates their membrane recruitment. Biochem. Biophys. Res. Commun. 350, 413-423 (2006) [33]). Several proteins which are involved in RNAi movement in drosophila, Arabidopsis, and C. elegans associate with late endosomes, or multivesicular body-like structures (e.g. vps41 and vps34) or ubiquitination (CG8184, UBA domain; SDE-5 (Hernandez-Pinzon, I. et al. SDE5, the putative homologue of a human mRNA export factor, is required for transgene silencing and accumulation of trans-acting endogenous siRNA. Plant J. 50, 140-148 (2007) [34]) or CG5382 (Saleh M C et al. Nat Cell Biol. 2006 8: 793 [94]), vps51 (Ring finger domains) [5].

### Example 2

### Sorting of GW182 into Multivesicular Bodies Controls MicroRNA Activity

### Material and Methods

Exosome Purification

Exosomes were purified by differential centrifugation as previously described (Raposo, G. et al. B lymphocytes secrete antigen-presenting vesicles. J Exp Med 183, 1161-72 (1996) [82]).

Confocal Microscopy

Images were captured with a Zeiss LSM 510 confocal microscope with 488 nm and 561 nm lasers and a 63× objective lens. Filters used were 500-530 nm (GFP) and 550-650 nm (N-Rh-PE, RFP).

Dynamic Light Scattering

Purified exosomes resuspended in DPBS (Invitrogen) were analyzed with a Zetasizer Nano S from Malvern Instruments (Malvern, UK) in 40 microL quartz cuvettes. Five measurements were performed in automatic mode after equilibration for 2 min at 20° C. Experimental data were processed with manufacturer's software in multiple narrow modes assuming spherical particles. Corrections for solvent refractive index (1.332) and viscosity (1.029) were employed.

Tests of miRNA Activity

To test miRNA activity, cells were transfected with 0.2 ug plasmid expressing the 3′UTR of ESR which contains a target site for miR-206, 2 μg of either plasmid expressing miR-206 or K12-4, and 10 nM siRNA. Luciferase activity was read 30 h later, using the Dual Luciferase Reporter Assay Kit (Promega, Madison, USA) on a Glo-Max Multi fluorescence reader (Promega). In other experiments, cells were transfected with 0.2 ug plasmid expressing Renilla luciferase with two linked let-7a target sites or mutated versions thereof (FIG. 11d, constructs Let-7a and b (Doench, J. G. & Sharp, P. A. Specificity of microRNA target selection in translational repression. Genes Dev 18, 504-11 (2004) [56]), 1 ug plasmid pGL3 expressing firefly luciferase, and 10 nM control or specific siRNA, and analyzed as above. Percent inhibition luciferase expression by specific miRNA was calculated as: e.g. tsg101 ([specific luc/control luc.]specific miRNA+tsg101 siRNA/[specific luc/control luc] control miRNA+tsg101 siRNA). Percent miRNA activity was calculated as 100−percent inhibition luciferase expression.

Statistics

All error bars shown represent standard error of the mean.

Antibodies

Antibodies were obtained as follows: Rabbit and mouse immunoglobuline G (Sigma-Aldrich, St. Quentin Fallavier, France), anti-CD63 (Santa Cruz Biotech, Santa Cruz, Calif., USA), anti-mono and poly-ubiquitinated proteins clone FK2 [Tebu-bio, Le Perray en Yvelines, France], anti-Dcp1a rabbit polyclonal (a kind gift of J. Lykke-Andersen, University of Colorado), anti-GW182 (serum 18033, a “index patient serum” taken from an autoimmune patient and characterized by Marvin Fritzler at the University of Calgary Canada originally in the article Eystatioy et al. Mol. Biol. Cell 2002 13: 1338 [95], the same derivation for anti-Ge-1 serum 106 following), anti-Ge-1 (serum 106), normal human serum (kind gifts of M. Fritzler, University of Calgary), anti-hrs (ab56468, ABCAM), anti-alix (clone 2H11, Santa Cruz).

Cell Culture and Loading with N-Rhodamine-PhosphatidylEthanolamine (N-Rh-PE)

The Mono-Mac6 cell line (DSMZ, Braunschweig, Germany ACC-124) was cultured in RPMI 1640 containing 10% FBS, non-essential amino acids (Invitrogen, Paris, France), and OPI media supplement (Sigma-Aldrich). Alternatively, Mono-Mac6 were cultured in X-Vivo-15 serum-free media (Lonza, Levallois, France). Hela cells were grown in DMEM supplemented with 5% FBS. Cells were loaded with 3 μM N-Rh-PE (Avanti Polar Lipids, Alabaster, Ala., USA) as described (Vidal, M., Mangeat, P. & Hoekstra, D. Aggregation reroutes molecules from a recycling to a vesicle-mediated secretion pathway during reticulocyte maturation. J Cell Sci 110 (Pt 16), 1867-77 (1997) [58]).

Plasmids and siRNA

Plasmids expressing GFP-hAgo2 (11590) (Addgene) (Jakymiw, A. et al. Disruption of GW bodies impairs mammalian RNA interference. Nat Cell Biol 7, 1267-74 (2005) [83]), YFP-CD82 (1819) (Addgene) (Sherer, N. M. et al. Visualization of retroviral replication in living cells reveals budding into multivesicular bodies. Traffic 4, 785-801 (2003) [57]), Gag-RFP (1814) (Addgene) ([57]), Sec6113-GFP (15108) (Addgene) and a 3′UTR containing two intact (11325) (Addgene) or mutated (11324) let-7a target sites 56 were obtained via Addgene (Cambridge, Mass., USA). Plasmids expressing GFP-GW182 (Ed Chan, University of Florida, USA), Dcp1a-RFP, Dcp1a-GFP, a dominant negative version of TIA-184 (Nancy Kedersha Brigham and Women's Hospital and Harvard Medical School, Boston, Mass., USA), and a constitutively active version of eIF2a (Randall Kaufmann, University of Michigan, Ann Arbor, Mich., USA) were kind gifts of Nancy Kedersha.

SiRNA were obtained from Qiagen (Courtaboeuf, France) unless otherwise indicated. * denotes an siRNA validated by the company to knockdown mRNA expression of the respective gene by at least 75%. These were: Hrs (CCGGAACGAGCCCAAGTACAA* (SEQ ID NO. 1), GCACGTCTTTCCAGAATTCAA* (SEQ ID NO. 2)), GW182 (TNRC6A, AAGAGCTTAACTCATCTTTAA* (SEQ ID NO. 3), ATGGATATGAACAGTATTAAA* (SEQ ID NO. 4)), Alix (AAGAGCTGTGTGTTGTTCAAT* (SEQ ID NO. 5), GAGGTACTTTATACTAACATA* (SEQ ID NO. 6)), vps36 (CCCGATCAATTGAGAATTTAT* (SEQ ID NO. 7), ACGGAGGTGTACTGCTTAGTA (SEQ ID NO. 8)), tsg101 (CAGTTTATCATTCAAGTGTAA* (SEQ ID NO. 9), ACCCGTTTAGATCAAGAAGTA* (SEQ ID NO. 10)), BIG2 (CGAUGAAAUUAAAGCAGAA (SEQ ID NO. 11)), PTPN23 (Ambion) AGUUUGUCCUGAAGAAUUAtt* (SEQ ID NO. 12), All Stars Negative Control siRNA (1027281). Knockdown of gene expression was confirmed by RT-PCR using the following primers: tsg101 (5′ GATACCCTCCCAATCCCAGT 3′ (SEQ ID NO. 13) and 5′ GTCACTGACCGCAGAGATGA 3′ (SEQ ID NO. 14)), vps36 (5′ CAGTGGCGTCATGGTAATTG 3′ (SEQ ID NO. 15) and 5′ CTGAGTCATCACGGCAAAGA 3′ (SEQ ID NO. 16)), alix (5′TGGCTGCAAAGCACTGTATC 3′ (SEQ ID NO. 17) and 5′ AGGGCACGATTGATTTTGTC 3′ (SEQ ID NO. 18)), BIG2 (5′ CAGGAGGTGGTGAAGGACAT 3′ (SEQ ID NO. 19) and 5′ CCCGTTGGTCTGTGAGTTT 3′ (SEQ ID NO. 20)), and hrs (5′ GGTCCAGGACACCTACCAGA 3′ (SEQ ID NO. 21) and 5′ AGTGGTGCTTACGGGTCATC 3′ (SEQ ID NO. 22)).

Small RNA Cloning and Library

Small RNA cloning was performed as described (Pfeffer, S., Lagos-Quintana, M. & Tuschl, T. Cloning of small RNA molecules. Curr Protoc Mol Biol Chapter 26, Unit 26 4 (2005) [85]) using 200 μg RNA from MonoMac6 cells or 2 μg RNA from MonoMac6-derived exosomes. Libraries were sequenced using 454 pyrosequencing technology (www.454.com) by GATC (Konstanz, Germany). Sequences were annotated using the following databases: genomic sequences were from the UCSC Genome Browser database (NCBI build 37, July 2007). tRNA, rRNA, sn-snoRNA, and scRNA sequences were extracted from release 158 of Genbank (Feb. 15, 2007). mRNA were identified using miRBase version 10.1.

mRNA-Dependent mRNA Sorting Experiments

Mono-Mac-6 cells were transfected with 1 μg reporter plasmids with target sites for Let-7a (FIG. 11d). After 24 h cells were washed three times in PBS and disrupted by 70 strokes of a Dounce homogenizer in 0.250 M sucrose, 78 mM KCl, 4 mM MgCl2, 8.4 mM CaCl2, 10 mM EGTA, 50 mM Hepes-NaOH pH 7.0. For membrane sorting experiments cell lysate was centrifuged at 1000 g (5 min) twice, and subsequently centrifuged at 16 000 g (45 min). The pellet was resuspended in 250 μL PBS and RNA was extracted in parallel with that from 250 μL supernatant using Trizol LS (Invitrogen). Exosome sorting experiments were performed identically and whole cells and purified exosomes were resuspended in 250 μL PBS for extraction of RNA using Trizol LS.

Bioinformatic Analysis of mRNA Sorting into Exosomes

Comparative mRNA microarrays detailing the relative enrichment of transcripts in glioblastoma cells compared to exosomes were recently published ([2]). Among 19619 probes detecting a signal above a background threshold of 200 units, 13504 (31.17%) were detected at higher levels in exosomes than cells. To evaluate the reduction of miRNA-targeted mRNA in exosomes we utilized experimentally validated targets (miRecord) for the 11 miRNA detected at high levels in glioblastoma cells and exosomes used for the microarrays 53. Analyzed identically, 6 (13.63%, expected 14, χ2=6.636, p=0.01) of 45 miRNA-repressed mRNA were over-represented in exosomes. Since no set of mRNA which are not targeted by miRNA is readily available we made use of a set of housekeeping genes, which tend to have shortened 3′UTR86 (Eisenberg, E. & Levanon, E. Y. Human housekeeping genes are compact. Trends Genet. 19, 362-5 (2003) [86]), potentially to minimize regulation by miRNA63. Of the housekeeping mRNA (1577 total probes), 568 (35.89%, expected 492, χ²=16.641, p<0.0001) were enriched in exosomes compared to cells.

### Results

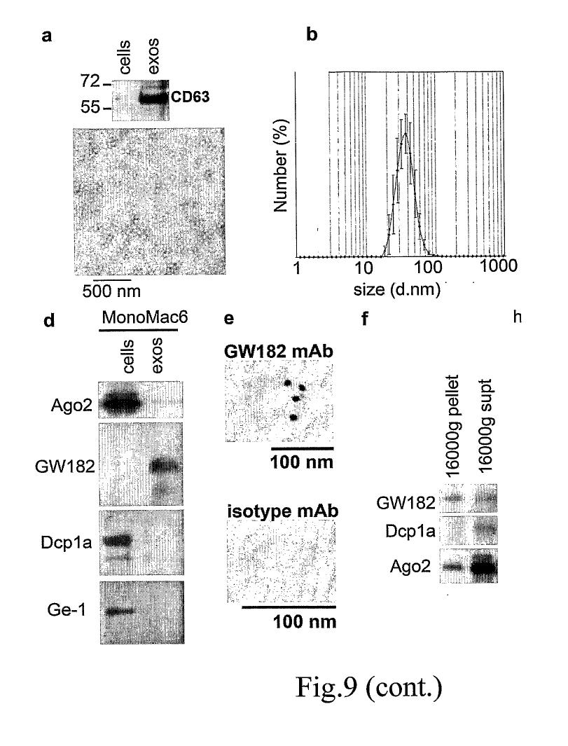

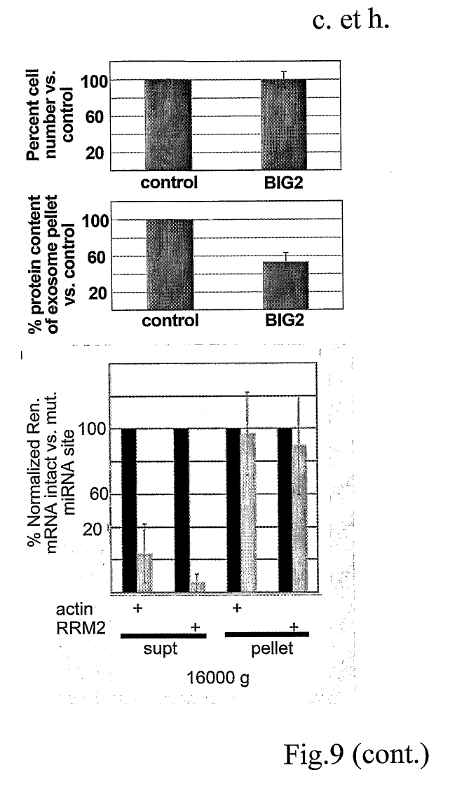

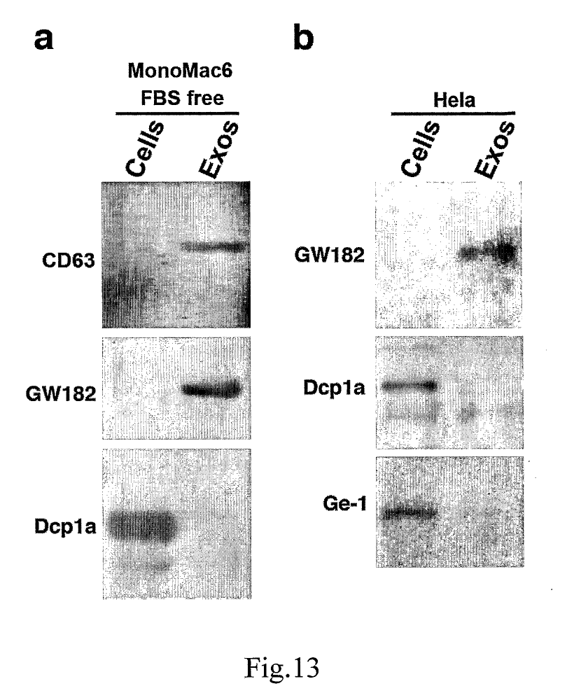

RNA extracted from secreted vesicles resembling exosomes ([52]) (50-100 nm in diameter) contain miRNAs (Skog, J. et al. Glioblastoma microvesicles transport RNA and proteins that promote tumour growth and provide diagnostic biomarkers. Nat Cell Biol 10, 1470-6 (2008) [53]). To test if such vesicles also contain proteins required for miRNA activity, we used cultured monocytes, known to secrete exosomes. We purified morphologically uniform vesicles forming a population homogenous in size consistent with exosomes (FIG. 9a-b). These were highly enriched in CD63, a known exosomal marker (FIG. 9a). Moreover, RNAi of Brefeldin-A-Inhibited Guanine nucleotide-exchange protein (BIG2), required for exosome release (Islam, A. et al. The brefeldin A-inhibited guanine nucleotide-exchange protein, BIG2, regulates the constitutive release of TNFR1 exosome-like vesicles. J Biol Chem 282, 9591-9 (2007) [54]), reduced vesicle yield by almost 50% (FIG. 9c). The purified, exosome-like material contained some Ago2, albeit much less than in whole-cell lysates, and was dramatically enriched in GW182 (FIG. 9d), required for miRNA function through binding to Ago2. Immuno-gold labeling and electron microscopy of permeabilized, purified vesicles further confirmed this GW182 enrichment (FIG. 9e). By contrast, the P-body component Dcp1a was barely detectable in secreted vesicles compared to whole-cell lysates, as was Ge-1, which interacts with Dcp1a in the decapping complex (Yu, J. H., Yang, W. H., Gulick, T., Bloch, K. D. & Bloch, D. B. Ge-1 is a central component of the mammalian cytoplasmic mRNA processing body. Rna 11, 1795-802 (2005) [55]) (FIG. 9d). Identical results were obtained with monocytes cultured in serum-free medium (excluding contribution of vesicles from bovine serum, (FIG. 13a) and with exosomes purified independently by third parties (S. Amigorena and colleagues, Institut Curie, Paris) from Hela (FIG. 13b) and ex vivo-derived dendritic cells (data not shown).

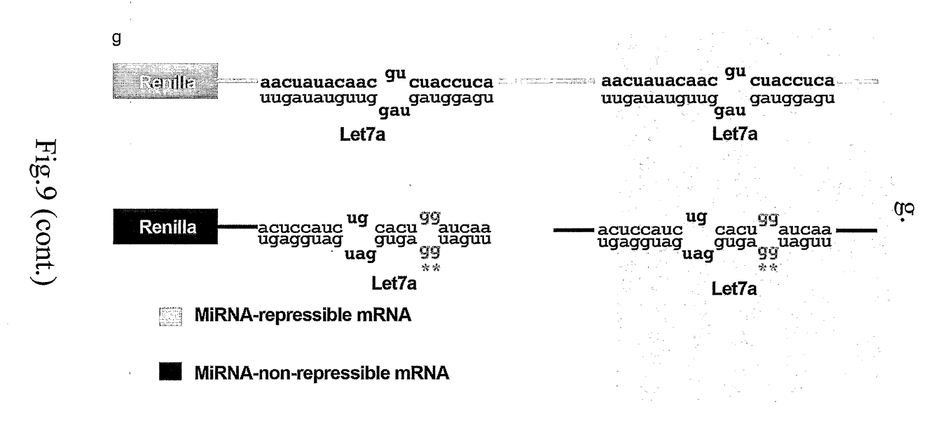

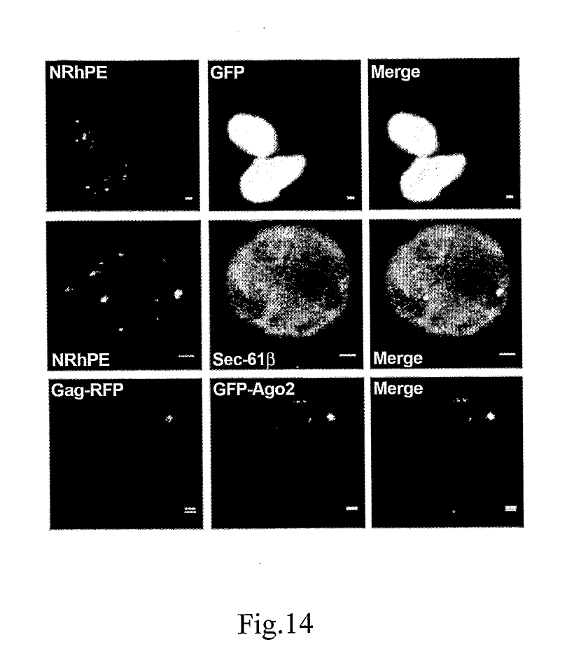

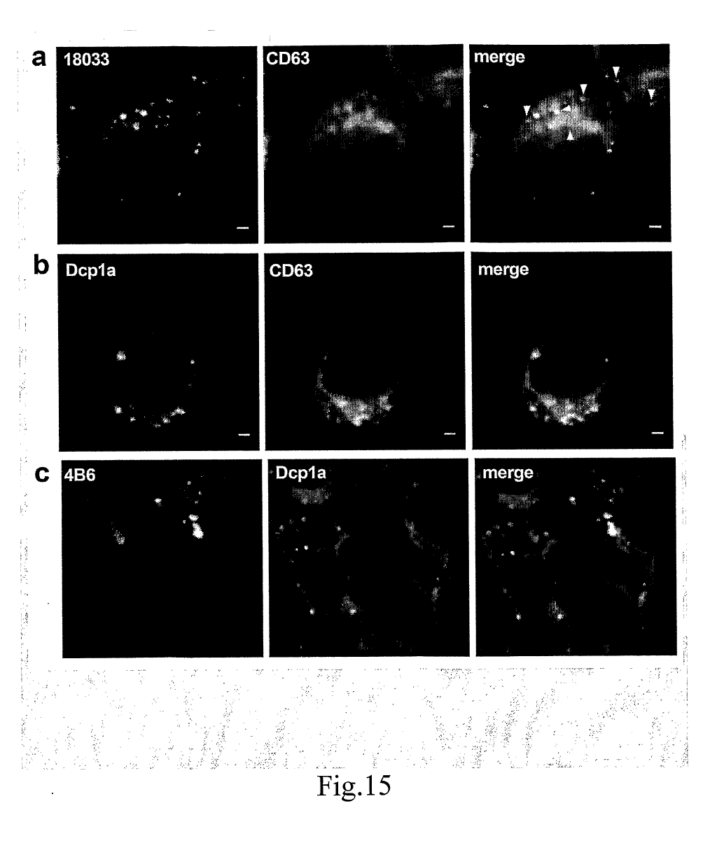

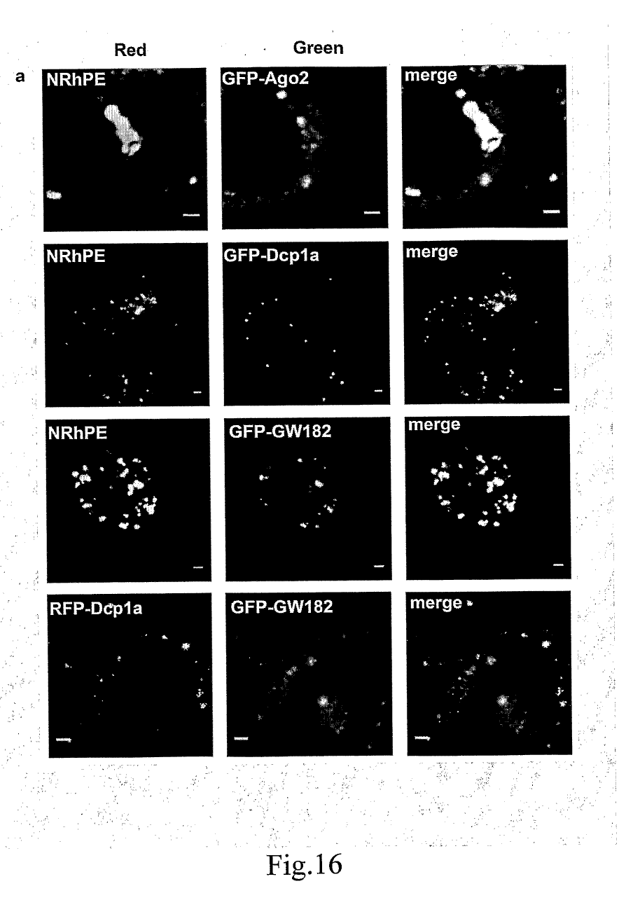

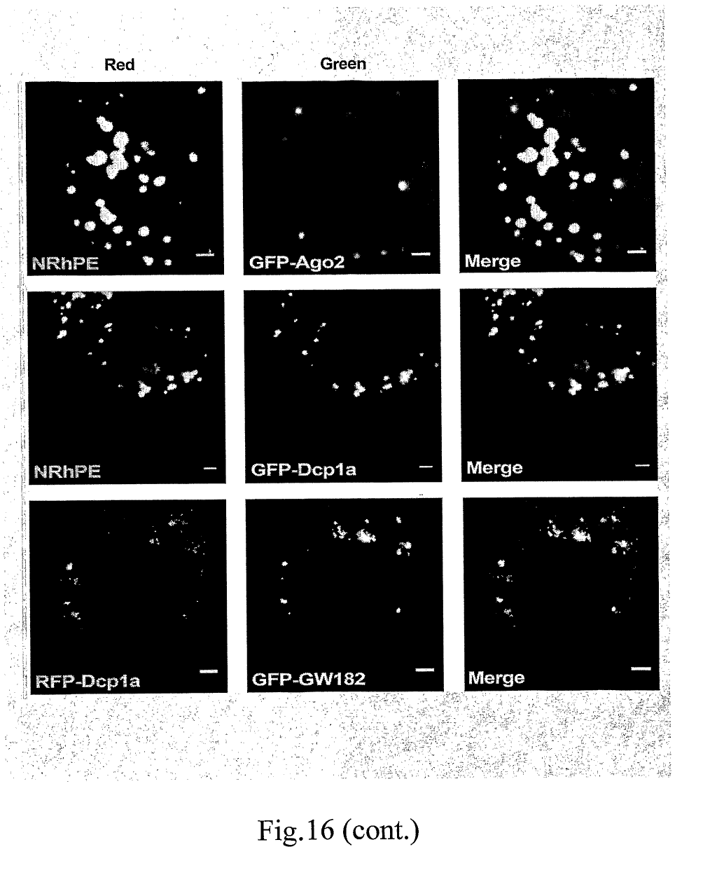

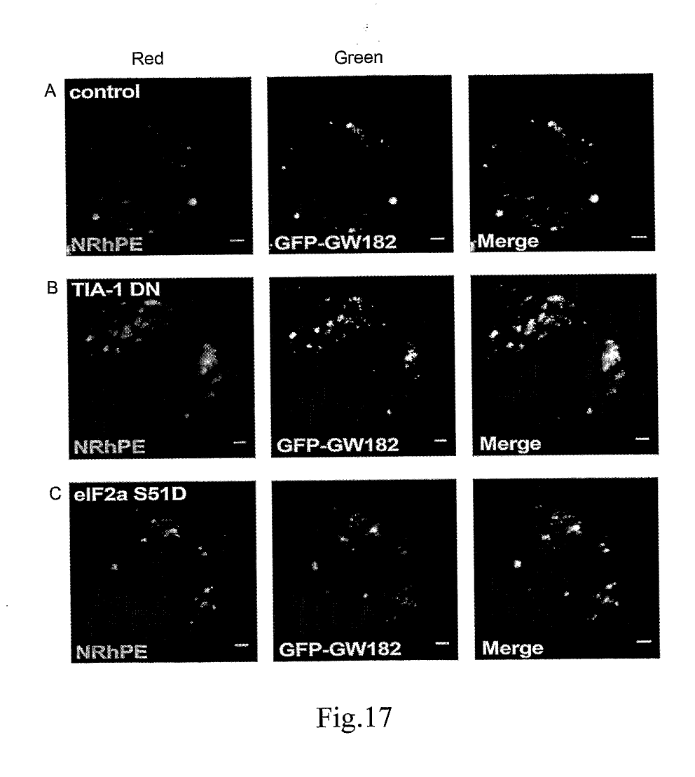

To be packaged into, or associated with exosomes, GW182 and GW182-bound Ago2 should interact with membranes. Indeed, much higher amounts of GW182 and Ago2 than Dcp1a were found in pellets as opposed to supernatants of whole-cell 16000 g fractions (FIG. 9f). To test whether miRNA-repressed mRNAs are also targeted to membranes, we used a plasmid expressing the Renilla mRNA fused to a 3′UTR containing two Let-7a target sites 5, or a negative control containing two seed mismatches (FIG. 9g). Strikingly, most Let-7a-repressed mRNAs were associated with membranes, compared to the control mRNAs (FIG. 9g), suggesting the existence of a membrane-associated miRNA-RISC. Since exosomes are secreted by MVB, we tested whether GW182- and Ago2-associated organelles might include MVB. We used N-Rhodamine-labeled lipid phosphatidylethanolamine (N-Rh-PE) because it is sorted to, and retained within, MVB (multivesicular bodies) MVB57. Moreover, auto-antibodies against PE, which in some circumstances selectively accumulates in MVB58, stain the same foci as GW182 auto-antibodies 59. Accurate MVB labeling in monocytes was confirmed by co-localization of N-Rh-PE with the MVB-associated tetraspanin protein CD82 (FIG. 10a; FIG. 14). GFP-tagged GW182 (GFP-GW182) formed punctuate structures, a majority of which co-localized with N-Rh-PE in 93% of monocytes tested (n=83) (FIG. 10b-c). A similar co-localization of GFP-Ago2 punctuate structures with N-Rh-PE was observed in 83% of cells (FIG. 10d), in agreement with Ago2 presence in membrane fractions (FIG. 9f). Moreover, GFP-Ago2 co-localized with the MVB-targeted HIV-1 Gag protein 57 (FIG. 14). By contrast, only 6% of cells exhibited co-localization between GFP-tagged Dcp1a, a P-body-specific marker, and N-Rh-PE (FIG. 10e-f). Likewise, GFP-GW182 and RFP-tagged Dcp1a (RFP-Dcp1a) co-localized in only 3% of cells (FIG. 10g-h). Immuno-fluorescence confirmed that endogenous GW182 foci co-localize more frequently with the MVB marker CD63 than do endogenous Dcp1a foci (FIG. 15) and similar, though less dramatic differences, were also observed between the respective localization of GFP-GW182, GFP-Ago2 and Dcp1a-GFP in N-Rh-PE-labeled Hela cells (FIG. 16). Agreeing with previous demonstrations that stress granules are GW182-free60, neither induction nor suppression of stress granules altered co-localization of GFP-GW182 with MVB in monocytes (FIG. 17). Therefore, Ago2 and GW182 foci co-localizing with MVB define subcellular structures distinct from P-bodies and stress granules. We deduce that at least two cellular pools of Ago2-associated RNA silencing components exist. One pool, defining ‘GW’ bodies, is GW182-rich, Dcp1a-poor and often associated with MVB. The second pool is non-membranous, Dcp1a-rich, GW182-poor, and identical to structures commonly defined as P-bodies.

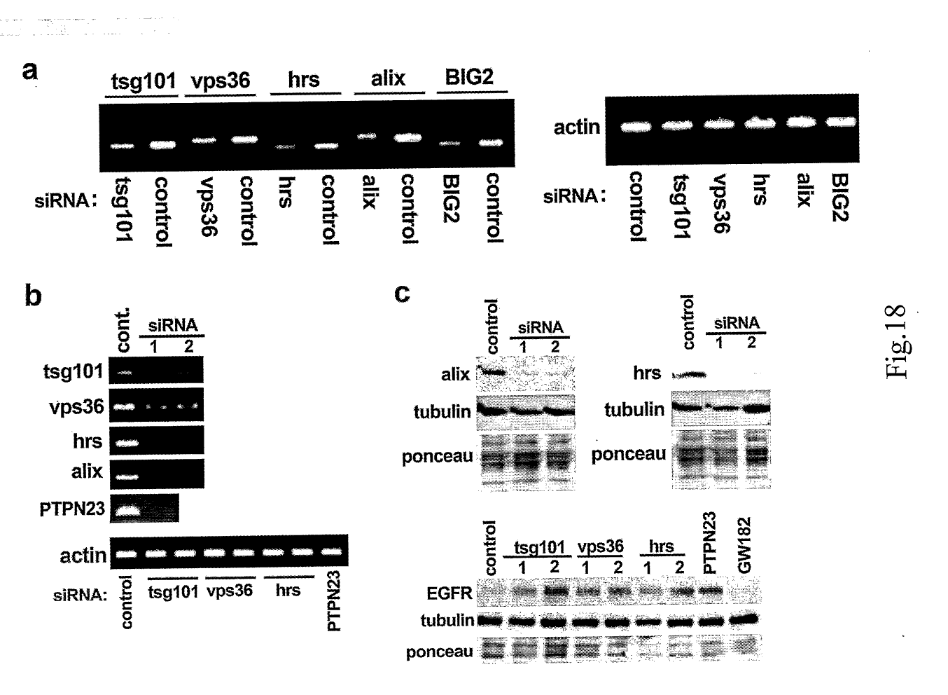

The relative co-localization of GW182, Ago2 and Dcp1a with MVB strikingly parallels their presence in membrane fractions and incorporation (or lack thereof) into secreted, exosome-like vesicles. MVB might, therefore, constitute functional sites of miRNA-mediated gene silencing. We first assayed the presence, in exosome-like vesicles, of mature miRNAs, as opposed to miRNA precursor transcripts, passenger strands, or miRNA degradation products, not always discriminated in previous qRT-PCR or DNA chip analyses ([53], Valadi, H. et al. Exosome-mediated transfer of mRNAs and microRNAs is a novel mechanism of genetic exchange between cells. Nat Cell Biol 9, 654-9 (2007) [61]). The 19-33 nt RNA fraction isolated from monocytic exosome-like vesicles was subjected to sequencing. Among the 6986 genome-matching sequences, 17% were known miRNAs (FIG. 11a). Agreeing with previous qRT-PCR studies, the cloning frequency ([53], Taylor, D. D. & Gercel-Taylor, C. MicroRNA signatures of tumor-derived exosomes as diagnostic biomarkers of ovarian cancer. Gynecol Oncol 110, 13-21 (2008) [62]) and length of mature miRNAs isolated from vesicles and whole cells were similar (FIG. 11b-c; data not shown); likewise, miRNA passenger strands (0.793% vs. 0.558%) and stem-loops (3.48% vs. 1.85%) were cloned at comparable, albeit much lower frequencies, in exosome-like and cellular fractions. Analysis of Let-7a, abundant in monocytic exosome-like vesicles (FIG. 11c), further indicated that membranes protect mature miRNAs against RNAse treatments (FIG. 18). Purified exosome-like vesicles thus contain single-stranded, mature miRNAs in addition to high levels of GW182 and low levels of Ago2.

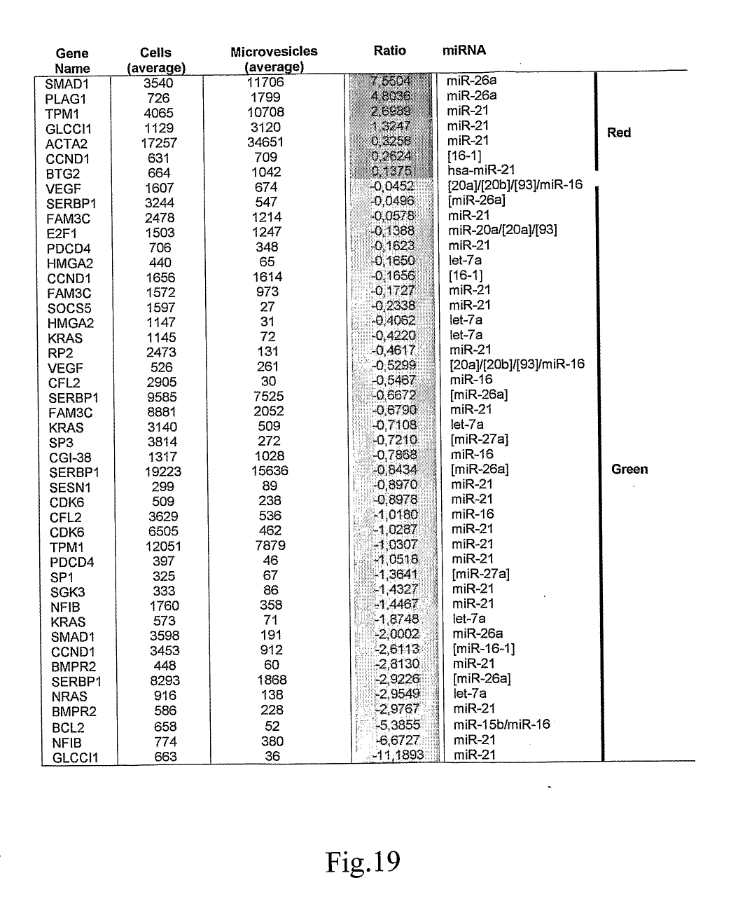

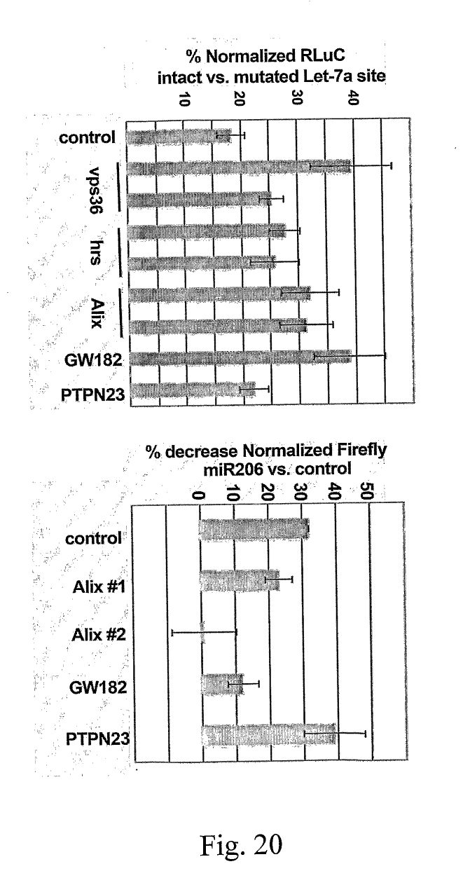

Having established that, like Ago2 and GW182, let-7a-repressed mRNAs are membrane-associated (FIG. 9f-g), we further tested their possible targeting to exosomes. Strikingly, however, let-7a-repressed mRNA were markedly underrepresented in purified exosome-like vesicles of monocytes (FIG. 11d). Comparing the whole-cell versus exosomal mRNA content of glioblastoma [53] similarly uncovered that known miRNA target transcripts are underrepresented in exosomes compared to all detected mRNAs (FIG. 11d, FIG. 19). By contrast, housekeeping gene mRNAs, less subject to miRNA-mediated repression (Farh, K. K. et al. The widespread impact of mammalian MicroRNAs on mRNA repression and evolution. Science 310, 1817-21 (2005) [63]), are enriched in exosomes (FIG. 11d). Under-representation of miRNA targets in exosomes is unlikely to result from miRNA-mediated mRNA decay, which usually affects a much smaller fraction of target mRNAs (if any) and occurs in DCP1a-associated P-bodies (Eulalio, A. et al. Target-specific requirements for enhancers of decapping in miRNA-mediated gene silencing. Genes Dev 21, 2558-70 (2007) [64]) distinct from MVB or secreted vesicles (FIGS. 9d and 10ef). Thus, while miRNA-repressed transcripts are enriched in GW182- and Ago2-associated membranous fractions, they seem selectively excluded from exosome-like vesicles. We so envisaged that a pool of GW182 specifically dissociates from membrane-bound, Ago-miRNA-mRNA silencing complexes to be sorted into MVB and further secreted or turned-over via the exosome/lysosome pathway.

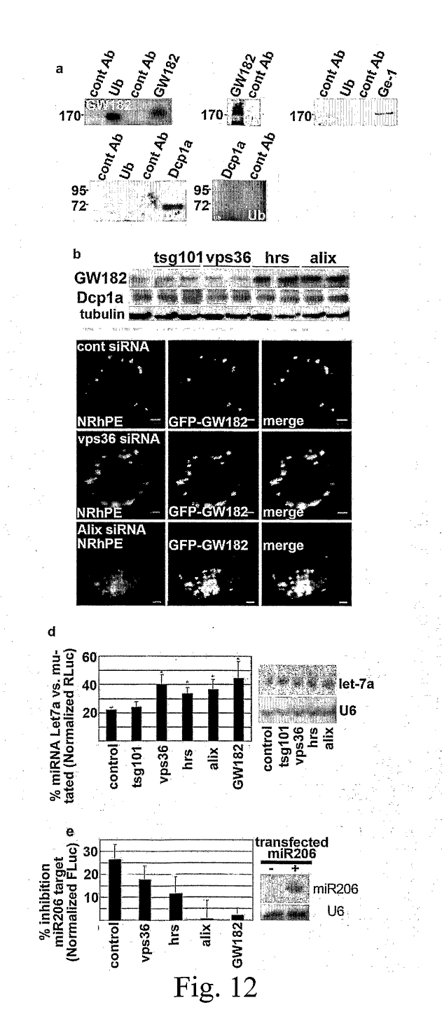

A major MVB sorting mechanism relies on recognition of ubiquitinated proteins by the ESCRT complex. Ago2 is purified with unknown ubiquitinated proteins (Matsumoto, M. et al. Large-scale analysis of the human ubiquitin-related proteome. Proteomics 5, 4145-51 (2005) [65]), and ubiquitin is found in some Ago complexes isolated by density (Hock, J. et al. Proteomic and functional analysis of Argonaute-containing mRNA-protein complexes in human cells. EMBO Rep 8, 1052-60 (2007) [66]). To determine if GW-body component are ubiquitinated and, thus, possibly sorted into MVB, silencing factors were immunoprecipitated from total protein extracts and their association with ubiquitinated proteins or direct ubiquitination tested. The anti-Ub antibody immunoprecipitated a protein reacting with the GW182 antiserum (albeit at a slightly lower size), and the anti-GW182 antibody immunoprecipitated a ubiquitinated protein consistent with the size of GW182 (FIG. 12a). Therefore, GW182 is ubiquitinated and/or interacts with ubiquitinated proteins. While predominantly recognizing GW182, the GW182 antiserum used also contains antibodies against Ge-1 (Bloch, D. B., Gulick, T., Bloch, K. D. & Yang, W. H. Processing body autoantibodies reconsidered. Rna 12, 707-9 (2006) [67]). However, in contrast to GW182, Ge-1 was not detected among ubiquitinated proteins, nor was Dcp1a (FIG. 12a), agreeing with their absence in secreted exosomes. ESCRT-dependent sorting of ubiquitinated GW182 into MVB was further supported by the results of siRNA-mediated knockdown of ESCRT components including vps36, tsg101, Alix (found in exosomes or required for normal MVB biogenesis/functions) and Hrs (necessary for intraluminal vesicle accumulation within MVB (Razi, M. & Futter, C. E. Distinct roles for Tsg101 and Hrs in multivesicular body formation and inward vesiculation. Mol Biol Cell 17, 3469-83 (2006) [68])). Indeed, Hrs and Alix silencing specifically increased the cellular content in GW182, but not Dcp1a, indicating a possible role for these two proteins in exosomal secretion and/or lysosomal degradation of GW182 (FIG. 12b). Noteworthy, while, as reported [18], RNAi of vps36 and Alix respectively increased MVB size and enhanced perinuclear MVB distribution, GFP-GW182 localization to MVB remained unaltered by these treatments (FIG. 12c) and upon tsg101 or Hrs knockdown (data not shown).

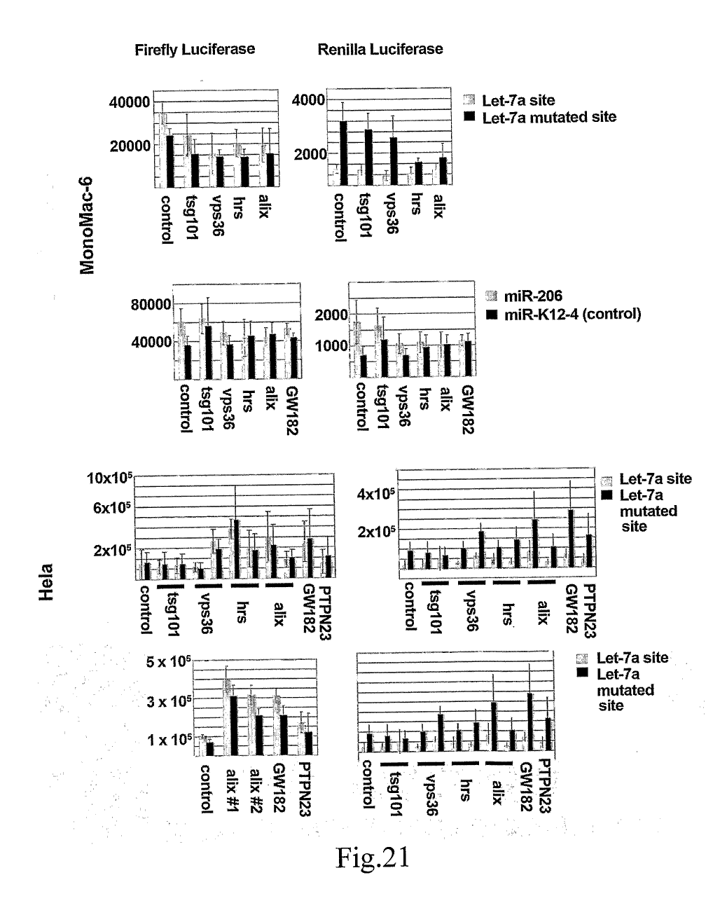

If GW182 sorting into MVB were relevant to RNA silencing, knockdown of ESCRT components would be expected to compromise miRNA activity. In a dual-luciferase assay, RNAi of Alix, Hrs and vps36 indeed inhibited let-7a activity in both monocytes (FIG. 12d) and Hela cells (two independent siRNA/gene, FIG. 21) to an extent comparable to siRNA-mediated knockdown of GW182A (˜20%, as observed by others (Liu, J. et al. A role for the P-body component GW182 in microRNA function. Nat Cell Biol 7, 1261-6 (2005) [70])). By contrast, knockdown of tsg101, which forms vacuolar domains within the early endosome [68], or PTPN23, required for EGFR degradation [20], gave little or no effect (FIG. 12d, FIG. 21). None of the above siRNA treatments affected let-7a accumulation (FIG. 12b, right panel). Similar experiments were carried out with a plasmid expressing miR-206 (or a control miRNA), which is absent from exosomes and monocytes (FIG. 12c). A second plasmid was transfected, expressing a firefly luciferase mRNA with the 3′UTR of the estrogen receptor-α transcript, a validated miR-206 target. As with let-7a, knockdown of Alix or Hrs, but not tsg101 or PTPN23, inhibited the specific activity of miR-206 (FIG. 12e, FIG. 21). We conclude that altering ESCRT integrity generally compromises miRNA functions, likely by interfering with the sorting of GW182 into MVB.

Sponge bodies, and ER-like compartments are examples of membranous compartments with which translationally-regulated mRNA or select P-body components associate (Decker, C. J. & Parker, R. CAR-1 and trailer hitch: driving mRNP granule function at the ER? J Cell Biol 173, 159-63 (2006) [72]). We demonstrate that specific miRNA pathway components, mature miRNAs, and miRNA-repressed transcripts physically and functionally congregate on cellular membranes, prominently including MVB. We propose, therefore, that the GW182 aggregates commonly detected in various mammalian cell types and previously defined as ‘GW’-bodies often, albeit not always, correspond to MVB. The limited co-localization between MVB and the P-body-specific component Dcp1a concurs with the incomplete association of GW182 with Dcp1a found by us and others (Buchet-Poyau, K. et al. Identification and characterization of human Mex-3 proteins, a novel family of evolutionarily conserved RNA-binding proteins differentially localized to processing bodies. Nucleic Acids Res 35, 1289-300 (2007) [73], Vasudevan, S., Tong, Y. & Steitz, J. A. Cell-cycle control of microRNA-mediated translation regulation. Cell Cycle 7, 1545-9 (2008) [74]). Additionally GW182, but not Dcp1a, partially co-localizes with FXR1 and Ago2 during miRNA-directed translation activation (Vasudevan, S. & Steitz, J. A. AU-rich-element-mediated upregulation of translation by FXR1 and Argonaute 2. Cell 128, 1105-18 (2007) [75]), agreeing with our finding that a significant portion of GFP-Ago2 aggregates localizes to GW-bodies in monocytes and Hela cells. Nonetheless, we consistently observed a fraction of Hela cells in which Dcp1a, GW182, and MVB clearly co-localize, which also supports observations made by others (Sen, G. L. & Blau, H. M. Argonaute 2/RISC resides in sites of mammalian mRNA decay known as cytoplasmic bodies. Nat Cell Biol 7, 633-6 (2005) [76]). One key element that might underlie these differences is that miRNA activities seem modulated in a cell-cycle-dependent manner in synchronization with GW-bodies, but not P-bodies [74], such that differences in cycle progression among cultured cells might generate significant labeling heterogeneity.

Although they are associated to MVB, miRNA-repressed mRNAs and a large fraction of Ago2 are excluded from exosomes. By contrast, GW182 is selectively enriched in those vesicles. Our interpretation entails that elevated dissociation rates of membrane-bound RNA-induced silencing complexes (RISC), containing Ago, miRNA, mRNA and GW182, are required in vivo for multiple rounds of mRNA repression, a known biochemical feature of RISC in vitro [26]. These high off-rates might be granted by specific- and possibly continuous-ESCRT-dependent removal of ubiquitinated GW182 molecules into MVB, allowing their subsequent secretion into exosomes and/or degradation in lysosomes. Implicit to this hypothesis, GW182 should be rate limiting in miRNA-mediated silencing, which is supported by the recent demonstration that Ago2 tethered to mRNA is no longer repressive if GW182 is knocked down (Li, S. et al. Identification of GW182 and its novel isoform TNGW1 as translational repressors in Ago2-mediated silencing. J Cell Sci 121, 4134-44 (2008) [78]). Unmaking and remaking of RISCs coupled to ESCRT-dependent sorting of GW182 further accommodates that a moderate fraction of Ago2 and mature miRNAs are also partitioned into exosomes, most likely because some miRNA-loaded Ago2 interacts directly with GW-repeats, recently identified as AGO anchors (E1-Shami, M. et al. Reiterated WG/GW motifs form functionally and evolutionarily conserved ARGONAUTE-binding platforms in RNAi-related components. Genes Dev 21, 2539-44 (2007) [79]). The model also explains why compromising ESCRT integrity perturbs miRNA functions without altering co-localization of GW182 to MVB, and why GW182 levels increase upon Alix or Hrs knockdown: reduced secretion/degradation would cause accumulation of GW182 at cellular membranes, concomitantly compromising the proposed turnover of silencing complexes. Recycling of miRNA complexes at MVB is also fully consistent with a contemporaneous study (see Lee et al.) suggesting that MVB trafficking pathways are required for efficient loading of Drosophila Agos with miRNAs or siRNAs. Distinct branches of ESCRT pathways control various MVB functions, often through paralogous factors. Hence, PTPN23 controls post-ligation degradation of EGFR, whereas Alix has little effect thereupon (Doyotte, A., Mironov, A., McKenzie, E. & Woodman, P. The Bro1-related protein HD-PTP/PTPN23 is required for endosomal cargo sorting and multivesicular body morphogenesis. Proc Natl Acad Sci USA 105, 6308-13 (2008) [71]). Inversely, Alix silencing compromises GW182 sorting to MVB and miRNA activities, whereas PTPN23 knockdown does not. Thus, a specific subset of ESCRT components, distinct from those controlling EGFR downregulation, seems involved in the processes reported here.

Identifying MVB as additional, previously uncharacterized, sites of miRNA action prompts the question as to whether a functional distinction is to be made between MVB- and P-body-associated miRNA activities. Our results show that translational inhibition or storage of repressed mRNA mostly occurs at membranous GW-bodies, while mRNA decay ensues in cytoplasmic P-bodies. Previous work in Drosophila cells indeed shows that GW182 can repress gene expression in the absence of mRNA decay mechanisms, and that GW182 can silence mRNA without a polyA tail (Eulalio, A., Huntzinger, E. & Izaurralde, E. GW182 interaction with Argonaute is essential for miRNA-mediated translational repression and mRNA decay. Nat Struct Mol Biol 15, 346-53 (2008) [80]). Suggestions have also been made that translational repression occurs independently of P-bodies since it persists, and is possibly enhanced, in cells depleted of Ge-1 and Dcp1a [13], meaning that the translational repression machinery might congregate, at least partly, at MVB to form GW-bodies. This may also explain why miRNA-mediated translational repression can operate in the absence of detectable P-bodies (Chu, C. Y. & Rana, T. M. Translation repression in human cells by microRNA-induced gene silencing requires RCK/p54. PLoS Biol 4, e210 (2006) [81]).

### Example 3

### Method for Determining the Delivery Rates and/or Efficiency of a siRNA, miRNA or Related Molecule to Target Organs or Cells

A first step of collection of serum, supernatant or body fluid from site draining tissue or cells targeted by siRNA or miRNA (e.g. urine sample for prostrate or kidney targeted siRNA) is realized, approximately 12 h to 4 days after treatment of animal or patient with siRNA/miRNA or inhibitor thereof.

Then the removal of cellular and other large material by low-speed centrifugation, at 100-400 g for 5 minutes is performed. Supernatant is recovered.

The removal of large debris, vesicles and other particulates by a second centrifugation, for example 8000-12 000 g for 30 minutes is performed. Alternatively, this step is substituted by size-exclusion filtration using for example a 0.22 μM, 0.45 μM or up to 1 μM filter cutoff. Material passing through the filter is collected.

A final centrifugation is used to pellet small vesicles or exosomes, at 70 000-120 000 g for 1 h.

Vesicles are further purified at any step (at the beginning, after step, 1, 2, 3, or 4) using antibodies or other molecules that bind molecules enriched on vesicles or exosomes, for example using anti-CD63, anti-MHC class I, or sphingomyelin-binding molecules attached to a bead or other easily purified structure.

RNA is isolated, by a method such as Trizol extraction. RNA precipitation is enhanced by addition of glycogen, yeast tRNA or other materials.

Specific mRNA targeted by siRNA/miRNA treatment or inhibitor thereof, (and potentially control RNA) is quantified by quantitative real-time PCR or other method with similar outcome.

Quantities of mRNA are normalized to a control RNA, or to a measure of exosome quantity (e.g. amount of sphingomyelin, CD63). Alternatively the siRNA/miRNA or inhibitor is directly quantified in vesicles.

Amount of specific mRNA is further compared to similar measurement performed before or after treatment of patient or animal with siRNA/miRNA or inhibitor thereof, or to animals or patients that were untreated or treated with placebo molecules, thereby allowing to determine the delivery rates and/or efficiency of a siRNA, miRNA or related molecule to target organs or cells. Alternatively, levels of specific mRNA are compared to levels of the same mRNA in cells, for example white blood cells, to which the siRNA is not delivered.

This protocol is also used to give a diagnosis or prognosis of a patient linked to expression levels or presence of miRNA or mRNA.

### Example 4

### Method for the Screening of Candidate Molecules for Diagnosis or Treatment

Molecules mimicking or inhibiting miRNA/siRNA can be screened for their targets and off-target effects using the invention.

Tissue or cells approximating the treatment conditions are treated with miRNA/siRNA or inhibitor.

After a period of about 8 h to 4 days, exosome-like vesicles are purified from tissue or cells (according to the protocol in example 3), and optimally total cells, and/or membrane fractions are prepared from the same tissues or cells.

Membrane fractions are prepared by lysing cells by a Dounce homogenization. Post-nuclear supernatants (about 1000 g 5 minutes) are subsequently centrifuged at approximately 10 000 g to 100 000 g. Pelleted material is then used as membrane fractions. Alternatively, centrifugation on density gradients, or isolation on electric gradients are used to isolation of membrane fractions.

RNA is isolated from the various samples, by a method such as Trizol extraction.

RNA quantities are analyzed, preferably by methods allowing analysis of a large number of RNA in parallel, such as Solexa sequencing or microarrays.

Ratios or other comparative expressions of each RNA are established among exosomes, total cells, and/or membrane fractions. Ratios indicate the targeting of a RNA by a small RNA. The decrease of a RNA in exosomes compared to cells and/or membrane fractions indicate that it is targeted by miRNA/siRNA. In some instances it is possible to determine miRNA/siRNA targets using only one of the sample types (exosome-like vesicles, total cells, membrane fractions)

The comparison of the ratios of each RNA in treated and untreated cells further increases confidence that a given RNA is targeted by miRNA/siRNA.

RNA presumably targeted by miRNA/siRNA from previous steps are examined for the presence of miRNA/siRNA target sites. The looking for matches of nucleotide 2-7 “seed region” with the small RNA sequence, is used to further enhance confidence that a given RNA is targeted by miRNA/siRNA.

Retained RNA are considered as targets of small RNA of interest.

### REFERENCE LIST

1. Eystathioy, T. et al. A phosphorylated cytoplasmic autoantigen, GW182, associates with a unique population of human mRNAs within novel cytoplasmic speckles. Mol. Biol. Cell. 13, 1338-1351 (2002).
2. Schneider, M. D. et al. Gawky is a component of cytoplasmic mRNA processing bodies required for early Drosophila development. J. Cell Biol. 174, 349-358 (2006).
3. Jakymiw, A. et al. Disruption of GW bodies impairs mammalian RNAinterference. Nat. Cell Biol. 7, 1267-1274 (2005).
4. Cikaluk, D. E. et al. GERp95, a membrane-associated protein that belongs to a family of proteins involved in stem cell differentiation. Mol. Biol. Cell. 10, 3357-3372 (1999).
5. Laurino, C. C. et al. Human autoantibodies to diacyl-phosphatidylethanolamine recognize a specific set of discrete cytoplasmicdomains. Clin. Exp. Immunol. 143, 572-584 (2006).
6. Vidal, M., Mangeat, P., & Hoekstra, D. Aggregation reroutes molecules from a recycling to a vesicle-mediated secretion pathway during reticulocytematuration. J. Cell Sci. 110, 1867-1877 (1997).
7. Kedersha, N. L., Gupta, M., Li, W., Miller, I., & Anderson, P. RNA-binding proteins TIA-1 and TIAR link the phosphorylation of eIF-2 alpha to the assembly of mammalian stress granules. J. Cell Biol. 147, 1431-1442 (1999).
8. Kedersha, N. & Anderson, P. Mammalian stress granules and processing bodies. Methods Enzymol. 431:61-81., 61-81 (2007).
9. Peschard, P. et al. Structural basis for ubiquitin-mediated dimerization and activation of the ubiquitin protein ligase Cbl-b. Mol. Cell. 27, 474-485 (2007).
10. Bloch, D. B., Gulick, T., Bloch, K. D., & Yang, W. H. Processing body autoantibodies reconsidered. RNA. 12, 707-709 (2006).
11. Cabezas, A., Bache, K. G., Brech, A., & Stenmark, H. Alix regulates cortical actin and the spatial distribution of endosomes. J. Cell Sci. 118, 2625-2635 (2005).
12. Doench, J. G. & Sharp, P. A. Specificity of microRNA target selection in translational repression. Genes Dev. 18, 504-511 (2004).
13. Adams, B. D., Furneaux, H., & White, B. A. The micro-ribonucleic acid (miRNA) miR-206 targets the human estrogen receptor-alpha (ERalpha) and represses ERalpha messenger RNA and protein expression in breast cancer cell lines. Mol. Endocrinol. 21, 1132-1147 (2007).
14 Buschow, S. I., Liefhebber, J. M., Wubbolts, R., & Stoorvogel, W. Exosomes contain ubiquitinated proteins. Blood Cells Mol. Dis. 35, 398-403 (2005).
15. Thery, C., Zitvogel, L., & Amigorena, S. Exosomes: composition, biogenesis and function. Nat. Rev. Immunol. 2, 569-579 (2002).
16. Islam, A. et al. The brefeldin A-inhibited guanine nucleotide-exchange protein, BIG2, regulates the constitutive release of TNFR1 exosome-like vesicles. J. Biol. Chem. 282, 9591-9599 (2007).
17. Valadi, H. et al. Exosome-mediated transfer of mRNAs and microRNAs is a novel mechanism of genetic exchange between cells. Nat. Cell Biol. 9, 654-659 (2007).
18. Morelli, A. E. et al. Endocytosis, intracellular sorting, and processing of exosomes by dendritic cells. Blood. 104, 3257-3266 (2004).
19. Voinnet, O, Non-cell autonomous RNA silencing. FEBS Lett. 579, 5858-5871 (2005).
20. Matsumoto, M. et al. Large-scale analysis of the human ubiquitin-related proteome. Proteomics. 5, 4145-4151 (2005).
21. E1-Shami, M. et al. Reiterated WG/GW motifs form functionally and evolutionarily conserved ARGONAUTE-binding platforms in RNAi-related components. Genes Dev. 21, 2539-2544 (2007).
22. Chim, S. S. et al. Detection and characterization of placental microRNAs in maternal plasma. Clin. Chem. 54, 482-490 (2008).
23. Behm-Ansmant, I. et al. mRNA degradation by miRNAs and GW182 requires both CCR4:NOT deadenylase and DCP1:DCP2 decapping complexes. Genes Dev. 20, 1885-1898 (2006).
24. Parry, D. H., Xu, J., & Ruvkun, G. A whole-genome RNAi Screen for C. elegans miRNA pathway genes. Curr. Biol. 17, 2013-2022 (2007).
25. Wilsch-Brauninger, M., Schwarz, H., & Nusslein-Volhard, C. A sponge-like structure involved in the association and transport of maternal products during Drosophila oogenesis. J. Cell Biol. 139, 817-829 (1997).
26. Irion, U. & St, J. D. bicoid RNA localization requires specific binding of an endosomal sorting complex. Nature. 445, 554-558 (2007).
27. Dvorak, A. M. & Morgan, E. S. The case for extending storage and secretion functions of human mast cell granules to include synthesis. Prog. Histochem. Cytochem. 37, 231-318 (2002).
28. Bhanji, R. A., Eystathioy, T., Chan, E. K., Bloch, D. B., & Fritzler, M. J. Clinical and serological features of patients with autoantibodies to GW/P bodies. Clin. Immunol. 125, 247-256 (2007).
29. Laroia, G., Sarkar, B., & Schneider, R. J. Ubiquitin-dependent mechanism regulates rapid turnover of AU-rich cytokine mRNAs. Proc. Natl. Acad. Sci. U.S.A. 99, 1842-1846 (2002).
30. Hock, J. et al. Proteomic and functional analysis of Argonaute-containing mRNA-protein complexes in human cells. EMBO Rep. 8, 1052-1060 (2007).
31. Dorrello, N. V. et al. 56K1- and betaTRCP-mediated degradation of PDCD4 promotes protein translation and cell growth. Science. 314, 467-471 (2006).
32. An, Q., Huckelhoven, R., Kogel, K. H., & van Bel, A. J. Multivesicular bodies participate in a cell wall-associated defence response in barley leaves attacked by the pathogenic powdery mildew fungus. Cell Microbiol. 8, 1009-1019 (2006).
33. Wang, T. & Hong, W. RILP interacts with VPS22 and VPS36 of ESCRT-II and regulates their membrane recruitment. Biochem. Biophys. Res. Commun. 350, 413-423 (2006).
34. Hernandez-Pinzon, I. et al. SDE5, the putative homologue of a human mRNA export factor, is required for transgene silencing and accumulation of trans-acting endogenous siRNA. Plant J. 50, 140-148 (2007).
35. Saleh, M. C. et al. The endocytic pathway mediates cell entry of dsiRNA to induce RNAi silencing. Nat. Cell Biol. 8, 793-802 (2006).
36. Segura, E., Guerin, C., Hogg, N., Amigorena, S., & Thery, C. CD8+ dendritic cells use LFA-1 to capture MHC-peptide complexes from exosomes in vivo. J. Immunol. 179, 1489-1496 (2007).
37. Zeelenberg, I. S. et al. Targeting tumor antigens to secreted membrane vesicles in vivo induces efficient antitumor immune responses. Cancer Res. 68, 1228-1235 (2008).
38. Morelli, A. E. et al. Endocytosis, intracellular sorting, and processing of exosomes by dendritic cells. Blood. 104, 3257-3266 (2004).
39. O'Connell, R. M., Taganov, K. D., Boldin, M. P., Cheng, G., & Baltimore, D. MicroRNA-155 is induced during the macrophage inflammatory response. Proc. Natl. Acad. Sci. U.S.A. 104, 1604-1609 (2007)
40. Pelchen-Matthews, A., Raposo, G., & Marsh, M. Endosomes, exosomes and Trojan viruses, Trends Microbiol. 12, 310-316 (2004).
41. Sullivan, R., Saez, F., Girouard, J., & Frenette, G. Role of exosomes in sperm maturation during the transit along the male reproductive tract. Blood Cells Mol. Dis. 35, 1-10 (2005).
42. Martin-Serrano, J. The role of ubiquitin in retroviral egress. Traffic. 8, 1297-1303 (2007).
43. Segura, E., Amigorena, S., & Thery, C. Mature dendritic cells secrete exosomes with strong ability to induce antigen-specific effector immune responses. Blood Cells Mol. Dis. 35, 89-93 (2005).
44. Lecellier, C. H. et al. A cellular microRNA mediates antiviral defense in human cells. Science. 308, 557-560 (2005).
45. Philips, J. A., Porto, M. C., Wang, H., Rubin, E. J., & Perrimon, N. ESCRT factors restrict mycobacterial growth. Proc. Natl. Acad. Sci. U.S. A. 105, 3070-3075 (2008).
46. Vidal, M., Mangeat, P., & Hoekstra, D. Aggregation reroutes molecules from a recycling to a vesicle-mediated secretion pathway during reticulocyte maturation. J. Cell Sci. 110, 1867-1877 (1997).
47. Jakymiw, A. et al. Disruption of GW bodies impairs mammalian RNA interference. Nat. Cell Biol. 7, 1267-1274 (2005).
48. Sherer, N. M. et al. Visualization of retroviral replication in living cells reveals budding into multivesicular bodies. Traffic. 4, 785-801 (2003).
49. Voeltz, G. K., Prinz, W. A., Shibata, Y., Rist, J. M., & Rapoport, T. A. A class of membrane proteins shaping the tubular endoplasmic reticulum. Cell. 124, 573-586 (2006).
50. Pfeffer, S. Identification of Virally Encoded MicroRNAs. Methods Enzymol. 427:51-63., 51-63 (2007).
51. Pfeffer, S., Lagos-Quintana, M., & Tuschl, T. Cloning of small RNA molecules. Curr. Protoc. Mol. Biol. Chapter 26:Unit 26.4., Unit (2005).
52. Thery, C., Zitvogel, L. & Amigorena, S. Exosomes: composition, biogenesis and function. Nat Rev Immunol 2, 569-79 (2002).
53. Skog, J. et al. Glioblastoma microvesicles transport RNA and proteins that promote tumour growth and provide diagnostic biomarkers. Nat Cell Biol 10, 1470-6 (2008).
54. Islam, A. et al. The brefeldin A-inhibited guanine nucleotide-exchange protein, BIG2, regulates the constitutive release of TNFR1 exosome-like vesicles. J Biol Chem 282, 9591-9 (2007).
55. Yu, J. H., Yang, W. H., Gulick, T., Bloch, K. D. & Bloch, D. B. Ge-1 is a central component of the mammalian cytoplasmic mRNA processing body. Rna 11, 1795-802 (2005).
56. Doench, J. G. & Sharp, P. A. Specificity of microRNA target selection in translational repression. Genes Dev 18, 504-11 (2004).
57. Sherer, N. M. et al. Visualization of retroviral replication in living cells reveals budding into multivesicular bodies. Traffic 4, 785-801 (2003).
58. Vidal, M., Mangeat, P. & Hoekstra, D. Aggregation reroutes molecules from a recycling to a vesicle-mediated secretion pathway during reticulocyte maturation. J Cell Sci 110 (Pt 16), 1867-77 (1997).
59. Laurino, C. C. et al. Human autoantibodies to diacyl-phosphatidylethanolamine recognize a specific set of discrete cytoplasmic domains. Olin Exp Immunol 143, 572-84 (2006).
60. Kedersha, N. et al. Stress granules and processing bodies are dynamically linked sites of mRNP remodeling. J Cell Biol 169, 871-84 (2005).
61. Valadi, H. et al. Exosome-mediated transfer of mRNAs and microRNAs is a novel mechanism of genetic exchange between cells. Nat Cell Biol 9, 654-9 (2007).
62. Taylor, D. D. & Gercel-Taylor, C. MicroRNA signatures of tumor-derived exosomes as diagnostic biomarkers of ovarian cancer. Gynecol Oncol 110, 13-21 (2008).
63. Farh, K. K. et al. The widespread impact of mammalian MicroRNAs on mRNA repression and evolution. Science 310, 1817-21 (2005).
64. Eulalio, A. et al. Target-specific requirements for enhancers of decapping in miRNA-mediated gene silencing. Genes Dev 21, 2558-70 (2007).
65. Matsumoto, M. et al. Large-scale analysis of the human ubiquitin-related proteome. Proteomics 5, 4145-51 (2005).
66. Hock, J. et al. Proteomic and functional analysis of Argonaute-containing mRNA-protein complexes in human cells. EMBO Rep 8, 1052-60 (2007).
67. Bloch, D. B., Gulick, T., Bloch, K. D. & Yang, W. H. Processing body autoantibodies reconsidered. Rna 12, 707-9 (2006).
68. Razi, M. & Futter, C. E. Distinct roles for Tsg101 and Hrs in multivesicular body formation and inward vesiculation. Mol Biol Cell 17, 3469-83 (2006).
69. Cabezas, A., Bache, K. G., Brech, A. & Stenmark, H. Alix regulates cortical actin and the spatial distribution of endosomes. J Cell Sci 118, 2625-35 (2005).
70. Liu, J. et al. A role for the P-body component GW182 in microRNA function. Nat Cell Biol 7, 1261-6 (2005).
71. Doyotte, A., Mironov, A., McKenzie, E. & Woodman, P. The Bro1-related protein HD-PTP/PTPN23 is required for endosomal cargo sorting and multivesicular body morphogenesis. Proc Natl Acad Sci USA 105, 6308-13 (2008).
72. Decker, C. J. & Parker, R. CAR-1 and trailer hitch: driving mRNP granule function at the ER? J Cell Biol 173, 159-63 (2006).
73. Buchet-Poyau, K. et al. Identification and characterization of human Mex-3 proteins, a novel family of evolutionarily conserved RNA-binding proteins differentially localized to processing bodies. Nucleic Acids Res 35, 1289-300 (2007).
74. Vasudevan, S., Tong, Y. & Steitz, J. A. Cell-cycle control of microRNA-mediated translation regulation. Cell Cycle 7, 1545-9 (2008).
75. Vasudevan, S. & Steitz, J. A. AU-rich-element-mediated upregulation of translation by FXR1 and Argonaute 2. Cell 128, 1105-18 (2007).
76. Sen, G. L. & Blau, H. M. Argonaute 2/RISC resides in sites of mammalian mRNA decay known as cytoplasmic bodies. Nat Cell Biol 7, 633-6 (2005).
77. Haley, B. & Zamore, P. D. Kinetic analysis of the RNAi enzyme complex. Nat Struct Mol Biol 11, 599-606 (2004).
78. Li, S. et al. Identification of GW182 and its novel isoform TNGW1 as translational repressors in Ago2-mediated silencing. J Cell Sci 121, 4134-44 (2008).
79. E1-Shami, M. et al. Reiterated WG/GW motifs form functionally and evolutionarily conserved ARGONAUTE-binding platforms in RNAi-related components. Genes Dev 21, 2539-44 (2007).
80. Eulalio, A., Huntzinger, E. & Izaurralde, E. GW182 interaction with Argonaute is essential for miRNA-mediated translational repression and mRNA decay. Nat Struct Mol Biol 15, 346-53 (2008).
81. Chu, C. Y. & Rana, T. M. Translation repression in human cells by microRNA-induced gene silencing requires RCK/p54. PLoS Biol 4, e210 (2006).
82. Raposo, G. et al. B lymphocytes secrete antigen-presenting vesicles. J Exp Med 183, 1161-72 (1996).
83. Jakymiw, A. et al. Disruption of GW bodies impairs mammalian RNA interference. Nat Cell Biol 7, 1267-74 (2005).
84. Kedersha, N. & Anderson, P. Mammalian stress granules and processing bodies. Methods Enzymol 431, 61-81 (2007).
85. Pfeffer, S., Lagos-Quintana, M. & Tuschl, T. Cloning of small RNA molecules. Curr Protoc Mol Biol Chapter 26, Unit 26 4 (2005).
86. Eisenberg, E. & Levanon, E. Y. Human housekeeping genes are compact. Trends Genet. 19, 362-5 (2003).
87. Navarro et al. Science 2008 321: 964
88. Yekta et al. Science, 2004, 304: 594
89. Wu et al. Mol. Cell. Biol. 2005, 25: 9198
90. Bhattacharyya et al. Cell 2006, 6:1111
91. Rossi Nat. Chem. Biol. 2007 3: 136
92. Maragkakis Nucleic Acids Research 2009 1-4
93. Schwamborn et al. Cell 2009 136: 913
94. Saleh M C et al. Nat Cell Biol. 2006 8: 793
95. Eystatioy et al. Mol. Biol. Cell 2002 13: 1338
96. Spizzo 2009 Cell 137: 586

1. A method for determining the delivery rates and/or efficiency of a siRNA, miRNA or related molecule to/in target organs or cells, comprising the measurement of levels, in the exosomes or vesicles of said target organs or cells, of said siRNA and/or miRNA and/or of mRNA targeted by said miRNA and/or by said siRNA.

2. A method according to claim 1, comprising the steps of:
(i) isolating exosomes or vesicles preferably from a bodily fluid of a patient previously treated with siRNA and/or miRNA,
(ii) measuring, in said exosomes, siRNA and/or miRNA and/or target mRNA levels,
(iii) possibly comparing said levels to a control,
then determining the delivery rates and/or efficiency of siRNA and/or miRNA in endogenous or therapeutics forms.

3. A method according to any of claim 1 or 2, wherein said bodily fluid is selected among blood products, urine, lung rinsings and saliva, other bodily fluids, or the supernatants of cultured cells.

4. A method according to claim 1 or 2, wherein said measurement of siRNA and/or miRNA and/or target mRNA levels is carried out by qRT-PCR, or by hybridization on microarray or other chip, or by hybridization on gel or membrane, or in solution.

5. A method according to claim 1, wherein said control is siRNA and/or miRNA and/or mRNA and/or other RNA and/or other molecule permitting to quantify vesicles, or a component thereof of a treated, non-treated, or control treated individual, animal or cells.

6. A method according to claim 2, wherein the steps (i) to (iii) are performed before and after, and eventually during siRNA and/or miRNA treatment.

7. A method according to claim 1, including determining the efficiency of delivery or activity of a siRNA and/or miRNA to target organs or cells.

8. A method according to claim 7, wherein the efficiency of delivery or activity of said siRNA and/or miRNA is recognized by a reduction or change in the levels of miRNA, siRNA or target mRNA in exosomes after treatment.

9. A method according to claims 7 or 8, wherein said step of determining the content of mRNA of whole cell is performed using a method selected among mRNA microarray, large-scale method of identifying mRNA or other RNA or DNA targeted by miRNA or siRNA, qRT-PCR, large-scale multigene approach.

10. A method according to claim 7 or 8, comprising the measurement of levels, in membrane fractions and in whole cells of said target organs or cells, of said siRNA and/or said miRNA and/or said mRNA targeted by said miRNA and/or by said siRNA.

11. A method according to claim 10, comprising the step of comparing the ratios of mi/siRNA targeted mRNA in exosomes, membrane fractions and whole cells.

12. A method according to claim 1, for identifying the target(s) of miRNA or siRNA therapeutics.

13. A method according to claim 1, for determining the efficiency of a treatment performed with siRNA and/or miRNA therapeutics, or with another molecule.

14. A method according to claim 13, wherein said treatment is performed with proteins, lipids, RNA or other molecules involved in the formation, interactions or activities of the multivesicular body (MVB) for modulating the activity and/or the cell-to-cell transfer of RNA, small RNA, for example miRNA, siRNA and piRNA, mRNA or non-coding RNA.

15. A method according to claim 14, wherein said proteins are selected from the group consisting of Alix, Hrs, vps36 (Vacuolar protein sorting associated protein 36), EAP30 (ELL-associated protein of 30 kDA, SNF8), EF1a (elongation factor 1a) and BIG2, hps4 (Hermansky-Pudlak syndrome 4), hps 1 (Hermansky-Pudlak syndrome 1), PRNP (prion protein), SCF ubiquitin ligase (Skp1-Cullin-F-box protein), Surfeit-4, V0 or V1 ATPase (V0 or V1 adenosine triphosphatase), vps41, COGC4 (Conserved Oligomeric Golgi Component 4), ATG3 (autophagy-related protein 3), ATG8, COG4, PI3K (Phosphatidylinositol 3-Kinase), NEDD4L (neural precursor cell expressed, developmentally down-regulated 4-like), ARFGEF4 (ADP-ribosylation factor Guanine nucleotide exchange factor-4 protein), CHML (Choroideremia-like), RAB10 (ras-related GTP-binding protein 10), RAB35, RALB (V-ral simian leukemia viral oncogene homolog B), RAPGEF6 (Rap Guanine Nucleotide exchange factor 6), SCD (stearoyl-CoA desaturase), GIPC1 (GIPC PDZ domain containing family, member 1), SCGB1D1 (secretoglobin, family 1D, member 1), UBE2M (Ubiquitin-conjugating enzyme E2M), USP10 (ubiquitin specific protease 10), EEF2 (eukaryotic translation elongation factor 2), LILRB1 (leukocyte immunoglobulin-like receptor, subfamily B), RAB36, RANBP2 (Ran Binding Protein 2), SFRP2 (secreted frizzled-related protein 2), SLC4A4 (Solute carrier family 4, sodium bicarbonate cotransporter, member 4), SMPD3 (sphingomyelin phosphodiesterase 3), Sphingomyelinase, sphingomyelin synthase 1, sphingomyelin synthase 2, Epopamil binding protein, usp22 (Ubiquitin-specific protease 22), trpc3 (Transient receptor potential cation channel, subfamily C, member 3), CLCN7 (Chloride channel 7), CTSC (cathepsin C), LAMR1 (Laminin receptor 1), RNF32 (Ring finger protein 32), ERI3 (Enhanced RNAi-3), HMGCR (3-hydroxy-3-methylglutaryl CoA reductase), NPC1 (Niemann-Pick disease, type C1), SLC6A4 (sodium-dependent serotonin transporter, solute carrier family 6 member 4), FAU, THEA (ACOT11, acyl-coenzyme A thioesterase 11), CKAP4, COG1-8 proteins, vps1-45 proteins, CHMP family proteins, sorting nexins, rab 5, 7, 9, 38, Arf2, Arf6, GGA1-3, sphingomyelin and sterol metabolism genes and drugs (e.g. GW4869, sphingomyelin esterase), drugs and genes affecting cholesterol or lipid raft partitioning and metabolism in relation to their involvement of sorting into MVB or exosomes, notably NPC1, HMGCR, and the statin classes of cholesterol lowering drugs.

16. A method for genotyping and/or characterizing the condition of a person, a tumor or a fetus, comprising a method according to claim 1.

17. A method for controlling the activity of miRNA or smalIRNA in an organism, a cell or a plant, comprising the administration in the organism of a protein, or a chemical that modify the activity of this protein, or a siRNA or miRNA or molecule related thereof targeting this protein, this protein being selected among Alix, Hrs, vps36 (Vacuolar protein sorting associated protein 36), EAP30 (ELL-associated protein of 30 kDA, SNF8), EF1a (elongation factor 1a) and BIG2, hps4 (Hermansky-Pudlak syndrome 4), hps 1 (Hermansky-Pudlak syndrome 1), PRNP (prion protein), SCF ubiquitin ligase (Skp1-Cullin-F-box protein), Surfeit-4, V0 or V1 ATPase (V0 or V1 adenosine triphosphatase), vps41, COGC4 (Conserved Oligomeric Golgi Component 4), ATG3 (autophagy-related protein 3), ATG8, COG4, PI3K (Phosphatidylinositol 3-Kinase), NEDD4L (neural precursor cell expressed, developmentally down-regulated 4-like), ARFGEF4 (ADP-ribosylation factor Guanine nucleotide exchange factor-4 protein), CHML (Choroideremia-like), RAB10 (ras-related GTP-binding protein 10), RAB35, RALB (V-ral simian leukemia viral oncogene homolog B), RAPGEF6 (Rap Guanine Nucleotide exchange factor 6), SCD (stearoyl-CoA desaturase), GIPC1 (GIPC PDZ domain containing family, member 1), SCGBID1 (secretoglobin, family 1D, member 1), UBE2M (Ubiquitin-conjugating enzyme E2M), USP10 (ubiquitin specific protease 10), EEF2 (eukaryotic translation elongation factor 2), LILRB1 (leukocyte immunoglobulin-like receptor, subfamily B), RAB36, RANBP2 (Ran Binding Protein 2), SFRP2 (secreted frizzled-related protein 2), SLC4A4 (Solute carrier family 4, sodium bicarbonate cotransporter, member 4), SMPD3 (sphingomyelin phosphodiesterase 3), Sphingomyelinase, sphingomyelin synthase 1, sphingomyelin synthase 2, Epopamil binding protein, usp22 (Ubiquitin-specific protease 22), trpc3 (Transient receptor potential cation channel, subfamily C, member 3), CLCN7 (Chloride channel 7), CTSC (cathepsin C), LAMR1 (Laminin receptor 1), RNF32 (Ring finger protein 32), ERI3 (Enhanced RNAi-3), HMGCR (3-hydroxy-3-methylglutaryl CoA reductase), NPC1 (Niemann-Pick disease, type C1), SLC6A4 (sodium-dependent serotonin transporter, solute carrier family 6 member 4), FAU, THEA (ACOT11, acyl-coenzyme A thioesterase 11), CKAP4, COG1-8 proteins, vps1-45 proteins, CHMP family proteins, sorting nexins, rab 5, 7, 9, 38, Arf2, Arf6, GGA1-3, sphingomyelin and sterol metabolism genes and drugs (e.g. GW4869, sphingomyelin esterase), drugs and genes affecting cholesterol or lipid raft partitioning and metabolism in relation to their involvement of sorting into MVB or exosomes, notably NPC1, HMGCR, and the statin classes of cholesterol lowering drugs.

18. A method of claim 1, including screening of candidate molecules for diagnosis or treatment.

19. Method according to claim 18, wherein said candidate molecules are selected among proteins or lipids involved in the formation of the multivesicular body (MVB) for modulating the activity and/or the cell-to-cell transfer of RNA, small RNA, for example miRNA, siRNA and piRNA, mRNA or non-coding RNA.

20. A kit comprising:
(a) means for isolating exosomes or vesicles from a bodily fluid,
(b) means for measuring, in said exosomes, siRNA and/or miRNA and/or target mRNA levels.


SEQUENCE LISTING (28 sequences)

SEQ ID NO: 1 - DNA, 21 bp, Artificial Sequence
  Description of Artificial Sequence Synthetic siRNA Hrs oligonucleotide
ccggaacgagcccaagtacaa

SEQ ID NO: 2 - DNA, 21 bp, Artificial Sequence
  Description of Artificial Sequence Synthetic siRNA Hrs oligonucleotide
gcacgtctttccagaattcaa

SEQ ID NO: 3 - DNA, 21 bp, Artificial Sequence
  Description of Artificial Sequence Synthetic siRNA GW182 oligonucleotide
aagagcttaactcatctttaa

SEQ ID NO: 4 - DNA, 21 bp, Artificial Sequence
  Description of Artificial Sequence Synthetic siRNA GW182 oligonucleotide
atggatatgaacagtattaaa

SEQ ID NO: 5 - DNA, 21 bp, Artificial Sequence
  Description of Artificial Sequence Synthetic siRNA Alix oligonucleotide
aagagctgtgtgttgttcaat

SEQ ID NO: 6 - DNA, 21 bp, Artificial Sequence
  Description of Artificial Sequence Synthetic siRNA Alix oligonucleotide
gaggtactttatactaacata

SEQ ID NO: 7 - DNA, 21 bp, Artificial Sequence
  Description of Artificial Sequence Synthetic siRNA vps36 oligonucleotide
cccgatcaattgagaatttat

SEQ ID NO: 8 - DNA, 21 bp, Artificial Sequence
  Description of Artificial Sequence Synthetic siRNA vps36 oligonucleotide
acggaggtgtactgcttagta

SEQ ID NO: 9 - DNA, 21 bp, Artificial Sequence
  Description of Artificial Sequence Synthetic siRNA tsg101 oligonucleotide
cagtttatcattcaagtgtaa

SEQ ID NO: 10 - DNA, 21 bp, Artificial Sequence
  Description of Artificial Sequence Synthetic siRNA tsg101 oligonucleotide
acccgtttagatcaagaagta

SEQ ID NO: 11 - RNA, 19 nt, Artificial Sequence
  Description of Artificial Sequence Synthetic siRNA BIG2 oligonucleotide
cgaugaaauuaaagcagaa

SEQ ID NO: 12 - DNA, 21 bp, Artificial Sequence
  Description of Artificial Sequence Synthetic siRNA PTPN23 oligonucleotide
aguuuguccugaagaauuatt

SEQ ID NO: 13 - DNA, 20 bp, Artificial Sequence
  Description of Artificial Sequence Synthetic primer tsg101
gataccctcccaatcccagt

SEQ ID NO: 14 - DNA, 20 bp, Artificial Sequence
  Description of Artificial Sequence Synthetic primer tsg101
gtcactgaccgcagagatga

SEQ ID NO: 15 - DNA, 20 bp, Artificial Sequence
  Description of Artificial Sequence Synthetic primer vps36
cagtggcgtcatggtaattg

SEQ ID NO: 16 - DNA, 20 bp, Artificial Sequence
  Description of Artificial Sequence Synthetic primer vps36
ctgagtcatcacggcaaaga

SEQ ID NO: 17 - DNA, 20 bp, Artificial Sequence
  Description of Artificial Sequence Synthetic primer alix
tggctgcaaagcactgtatc

SEQ ID NO: 18 - DNA, 20 bp, Artificial Sequence
  Description of Artificial Sequence Synthetic primer alix
agggcacgattgattttgtc

SEQ ID NO: 19 - DNA, 20 bp, Artificial Sequence
  Description of Artificial Sequence Synthetic primer BIG2
caggaggtggtgaaggacat

SEQ ID NO: 20 - DNA, 19 bp, Artificial Sequence
  Description of Artificial Sequence Synthetic primer BIG2
cccgttggtctgtgagttt

SEQ ID NO: 21 - DNA, 20 bp, Artificial Sequence
  Description of Artificial Sequence Synthetic primer hrs
ggtccaggacacctaccaga

SEQ ID NO: 22 - DNA, 20 bp, Artificial Sequence
  Description of Artificial Sequence Synthetic primer hrs
agtggtgcttacgggtcatc

SEQ ID NO: 23 - RNA, 22 nt, Artificial Sequence
  Description of Artificial Sequence Synthetic siRNA GFP 1022064 oligonucleotide
gaacuucagggucagcuugccg

SEQ ID NO: 24 - DNA, 21 bp, Artificial Sequence
  Description of Artificial Sequence Synthetic oligonucleotide
tagcagcacgtaaatattggc

SEQ ID NO: 25 - RNA, 21 nt, Artificial Sequence
  Description of Artificial Sequence Synthetic oligonucleotide
aacuauacaacgucuaccuca

SEQ ID NO: 26 - RNA, 22 nt, Artificial Sequence
  Description of Artificial Sequence Synthetic oligonucleotide
ugagguaguagguuguauaguu

SEQ ID NO: 27 - RNA, 21 nt, Artificial Sequence
  Description of Artificial Sequence Synthetic oligonucleotide
acuccaucugcacuggaucaa

SEQ ID NO: 28 - RNA, 22 nt, Artificial Sequence
  Description of Artificial Sequence Synthetic oligonucleotide
uugauggaguggaugauggagu